(19) United States
(12) Patent Application Publication
(10) Pub. No.: US 2021/0189367 A1
(43) Pub. Date: Jun. 24, 2021

(54) Inducible Cell Receptors for Cell-Based Therapeutics

(71) Applicants: Senti Biosciences, Inc., South San Francisco, CA (US); Trustees of Boston University, Boston, MA (US)

(72) Inventors: Philip Janmin Lee, South San Francisco, CA (US); Timothy Kuan-ta Lu, South San Francisco, CA (US); Russell Morrison Gordley, South San Francisco, CA (US); Wilson Wong, Brookline, MA (US); John Ngo, Cambridge, MA (US)

(21) Appl. No.: 16/771,510

(22) PCT Filed: Dec. 11, 2018
(86) PCT No.: PCT/US2018/065044

§ 371 Date: Jun. 10, 2020

Related U.S. Application Data
(60) Provisional application No. 62/597,191, filed on Dec. 11, 2017.
(60) Provisional application No. 62/597,212, filed on Dec. 11, 2017.

(51) Int. Cl. C12N 9/50; C07K 14/705; C12N 5/0783; A61K 35/17; C07K 16/32; C07K 16/28

(52) U.S. Cl. CPC C12N 9/506; C12Y 304/21098; C07K 14/70503; C12N 5/0636; A61K 35/17; A61K 38/00; C07K 16/2803; C07K 2319/30; C07K 2319/33; C07K 2319/02; C07K 2319/03; C07K 16/32

(57) ABSTRACT

The present disclosure provides inducible cell receptors and therapeutic cells comprising the inducible cell receptors. Further provided are methods of preparing the therapeutic cells and methods of treating a subject by administering the therapeutic cells and regulating activity of (e.g. activating and/or inactivating) the cell receptors.

### CROSS-REFERENCE TO RELATED APPLICATIONS

This application claims the benefit of U.S. Provisional Application No. 62/597,191, filed Dec. 11, 2017 and U.S. Provisional Application No. 62/597,212, filed Dec. 11, 2017, each of which is hereby incorporated by reference in its entirety

### SEQUENCE LISTING

The instant application contains a Sequence Listing which has been submitted electronically in ASCII format and is hereby incorporated by reference in its entirety. Said ASCII copy, created on Dec. 20, 2018, is named STB-005WOUS_SL.txt and is 50,869 bytes in size.

### BACKGROUND

Chimeric antigen receptors (CARs) enable targeted in vivo activation of immune T cells. These recombinant membrane receptors have an antigen-binding domain and one or more signaling domains (e.g., T cell activation domains). These special receptors allow the T cells to recognize and attach to a specific protein antigen on tumor cells. Recent results of clinical trials with chimeric receptor-expressing T cells have provided compelling support of their utility as agents for cancer immunotherapy (Pule et al., Nat. Med. (14):1264-1270 (2008); Maude et al., N Engl J Med. (371):1507-17 (2014); Brentjens et al., Sci Transl Med. (5):177ra38 (2013)).

However, despite these promising results, a number of side effects associated the CAR T-cell therapeutics were identified, raising significant safety concerns. The side effects include cytokine release syndrome (CRS)—a reversible yet potentially life-threatening condition mediated by the release of interleukin-6, tumor necrosis factor-α, and interferon-γ following immune cell activation—and tumor lysis syndrome—the sudden release of cellular contents into the bloodstream following tumor cell lysis. Furthermore, the long-term presence of CAR T in patients can induce mutagenesis, possibly in the CAR construct inserted into the CAR T cell and B cell apalasia, reducing their immune responses in long terms.

Therefore, there is a need for development of safer CAR T-cell for therapeutic use.

### SUMMARY

The present disclosure provides inducible cell receptors (e.g., CARs) and methods of regulating activity of the cell receptors that can be used for cell therapies with reduced side effects and enhanced safety. The inducible cell receptors can be configured as OFF switches (so that they can be selectively inactivated) or as ON switches (so that they can be selectively activated). These cellular switches can be used to regulate receptor activities in cell therapies to tune receptor activity.

In an aspect, the present disclosure provides a single-chain CAR with an OFF switch. In some embodiments, the receptor is a fusion protein comprising: a. a chimeric antigen receptor (CAR) comprising (a) an extracellular protein binding domain, and (b) a first intracellular signaling domain, and (c) a transmembrane domain located between the extracellular protein binding domain and the first intracellular signaling domain; and b. a self-excising degron operably linked to the CAR and comprising (a) a repressible protease, (b) a cognate cleavage site, and (c) a degradation sequence.

In some embodiments, the CAR further comprises a second intracellular signaling domain. In some embodiments, the CAR further comprises a third intracellular signaling domain.

In some embodiments, the extracellular protein binding domain is an antibody, an antigen-binding fragment thereof, F(ab), F(ab′), a single chain variable fragment (scFv), or a single-domain antibody (sdAb). In some embodiments, the extracellular protein binding domain comprises a ligand-binding domain. In some embodiments, the ligand-binding domain is a domain from a receptor, wherein the receptor is selected from the group consisting of TCR, BCR, a cytokine receptor, RTK receptors, serine/threonine kinase receptors, hormone receptors, immunoglobulin superfamily receptors, and TNFR-superfamily of receptors. In some embodiments, the receptor is a cytokine receptor selected from IL-1, IL-10, and IL-7, TGF-beta receptor, PD-1 or OX40.

In some embodiments, the self-excising degron is located at the C-terminus of the CAR. In some embodiments, the self-excising degron comprises the cognate cleavage site, the repressible protease, and the degradation sequence physically linked to one another in the sequential order from the N-terminus to the C-terminus. In some embodiments, the self-excising degron comprises the repressible protease, the cognate cleavage site, and the degradation sequence physically linked to one another in the sequential order from the N-terminus to the C-terminus.

In some embodiments, the fusion protein further comprises a protease inhibitor bound to the repressible protease. In some embodiments, the fusion protein further comprises a first recruitment domain.

In another aspect, the present disclosure provides a single-chain CAR with an ON switch. In some embodiments, the present disclosure provides a fusion protein comprising a chimeric antigen receptor (CAR) comprising (a) an extracellular protein binding domain, (b) a first intracellular signaling domain, and (c) a transmembrane domain located between the extracellular protein binding domain and the first intracellular signaling domain, (d) a repressible protease, and (e) a cognate cleavage site of the repressible protease.

In some embodiments, the CAR further comprises a second intracellular signaling domain. In some embodiments, the CAR further comprises a third intracellular signaling domain.

In some embodiments, the extracellular protein binding domain is an antibody, an antigen-binding fragment thereof, F(ab), F(ab′), a single chain variable fragment (scFv), or a single-domain antibody (sdAb). In some embodiments, the extracellular protein binding domain comprises a ligand-binding domain. In some embodiments, the ligand-binding domain is a domain from a receptor, wherein the receptor is selected from the group consisting of TCR, BCR, a cytokine receptor, RTK receptors, serine/threonine kinase receptors, hormone receptors, immunoglobulin superfamily receptors, and TNFR-superfamily of receptors. In some embodiments, the receptor is a cytokine receptor selected from IL-1, IL-10, and IL-7, TGF-beta receptor, PD-1 or OX40.

In some embodiments, the cognate cleavage site is located: a. between the transmembrane domain and the first intracellular signaling domain; b. between the extracellular protein binding domain and the transmembrane domain; c. between the first intracellular signaling domain and the second intracellular signaling domain; or d. between the second intracellular signaling domain and the third intracellular signaling domain.

In some embodiments, a. the cognate cleavage site and the repressible protease are physically linked to one another in the sequential order from the N-terminus to the C-terminus; or b. the repressible protease and the cognate cleavage site are physically linked to one another in the sequential order from the N-terminus to the C-terminus.

In some embodiments, the repressible protease is located at the C-terminus of the CAR.

In some embodiments, the CAR further comprises a ligand operably linked to the ligand-binding domain and the cognate cleavage site is located between the ligand-binding domain and the ligand. In some embodiments, the repressible protease and the cognate cleavage site are physically linked to one another.

In some embodiments, the fusion protein further comprises a protease inhibitor bound to the repressible protease.

In one aspect, the present disclosure provides a fusion protein comprising a chimeric antigen receptor (CAR) comprising from the C-terminus to the N-terminus: (a) a first intracellular signaling domain, (b) a repressible protease, (c) a cognate cleavage site of the repressible protease, (d) one or more additional intracellular signaling domains, (e) a transmembrane domain, and (f) an extracellular protein binding domain.

In another aspect, the present disclosure provides a fusion protein comprising a chimeric antigen receptor (CAR) comprising from the C-terminus to the N-terminus: (a) a repressible protease, (b) a first intracellular signaling domain, (c) a cognate cleavage site of the repressible protease, (d) one or more additional intracellular signaling domains, (e) a transmembrane domain, and (f) an extracellular protein binding domain.

In some embodiments, the CAR further comprises a spacer domain located between the extracellular protein binding domain and the transmembrane domain.

In another aspect, the present disclosure provides a multi-chain CAR with an OFF switch. In some embodiments, the present disclosure provides a composition of such inducible cell receptors comprising two fusion proteins—a. a first fusion protein comprising: (a) an extracellular protein binding domain, and (b) a first recruitment domain; and b. a second fusion protein comprising a chimeric antigen receptor (CAR), wherein the CAR comprises: (a) a second recruitment domain, (b) a transmembrane domain, (c) a first intracellular signaling domain, and a self-excising degron operably linked to the CAR, wherein the self-excising degron comprises (i) a repressible protease, (ii) a cognate cleavage site, and (iii) a degradation sequence.

In some embodiments, (a) the first fusion protein is a soluble protein; (b) the first fusion protein is a membrane-bound protein comprising a transmembrane domain, and the first recruitment domain is located between the extracellular protein binding domain and the transmembrane domain; or (c) the first fusion protein is a membrane-bound protein comprising a transmembrane domain, and the transmembrane domain is located between the first recruitment domain and the extracellular protein binding domain.

In some embodiments, (a) the CAR comprises from the N-terminus to the C-terminus the second recruitment domain, the transmembrane domain, and the first intracellular signaling domain; (b) the CAR comprises from the N-terminus to the C-terminus the transmembrane domain, the second recruitment domain, and the first intracellular signaling domain; or (c) the CAR comprises from the N-terminus to the C-terminus the transmembrane domain, the first intracellular signaling domain, and the second recruitment domain.

In some embodiments, the CAR further comprises a second intracellular signaling domain, optionally wherein the second intracellular signaling domain is located N-terminal to the first intracellular signaling domain or is located C-terminal to the first intracellular signaling domain.

In some embodiments, the CAR further comprises a second extracellular protein binding domain.

In some embodiments, the extracellular protein binding domain or the second extracellular protein binding domain is an antibody, an antigen-binding fragment thereof, F(ab), F(ab′), a single chain variable fragment (scFv), or a single-domain antibody (sdAb).

In some embodiments, the extracellular protein binding domain or the second extracellular protein binding domain comprises a ligand-binding domain. The ligand-binding domain can be a domain from a receptor, wherein the receptor is selected from the group consisting of TCR, BCR, a cytokine receptor, RTK receptors, serine/threonine kinase receptors, hormone receptors, immunoglobulin superfamily receptors, and TNFR-superfamily of receptors. In some embodiments, the receptor is a cytokine receptor selected from IL-1, IL-10, and IL-7, TGF-beta receptor, PD-1 or OX40.

In some embodiments, the self-excising degron is located at the C-terminus of the CAR.

In some embodiments, the self-excising degron comprises: (a) the cognate cleavage site, the repressible protease, and the degradation sequence physically linked to one another in the sequential order from the N-terminus to the C-terminus; or (b) the repressible protease, the cognate cleavage site, and the degradation sequence physically linked to one another in the sequential order from the N-terminus to the C-terminus.

In some embodiments, the first protein further comprises a second self-excising degron, wherein the second self-excising degron comprises (i) a second repressible protease, (ii) a second cognate cleavage site, and (iii) a second degradation sequence operably linked to one another.

In some embodiments, the first protein and the second protein are bound through the first recruitment domain and the second recruitment domain.

In some embodiments, the composition further comprises a protease inhibitor bound to the repressible protease.

In another aspect, the present disclosure provides a composition of inducible cell receptors comprising two fusion proteins—a. a first fusion protein comprising: (a) an extracellular protein binding domain, (b) a first recruitment domain, (c) a cognate cleavage site, and (d) a degradation sequence, and b. a second fusion protein comprising: (a) a transmembrane domain, (b) a second recruitment domain, and (c) a repressible protease.

In some embodiments, the cognate cleavage site and the degradation sequence are physically linked to one another. In some embodiments, the cognate cleavage site and the degradation sequence are located at the C-terminus of the first fusion protein. In some embodiments, the repressible protease is located at the C-terminus of the second fusion protein.

In some embodiments, the first fusion protein further comprises a first intracellular signaling domain. In some embodiments, the second fusion protein further comprises a second intracellular signaling domain.

In some embodiments, the second fusion protein further comprises a second extracellular protein binding domain.

In some embodiments, the extracellular protein binding domain or the second extracellular protein binding domain is an antibody, an antigen-binding fragment thereof, F(ab), F(ab′), a single chain variable fragment (scFv), or a single-domain antibody (sdAb).

In some embodiments, the extracellular protein binding domain or the second extracellular protein binding domain comprises a ligand-binding domain. In some embodiments, the ligand-binding domain is a domain from a receptor, wherein the receptor is selected from the group consisting of TCR, BCR, a cytokine receptor, RTK receptors, serine/threonine kinase receptors, hormone receptors, immunoglobulin superfamily receptors, and TNFR-superfamily of receptors. In some embodiments, the receptor is a cytokine receptor selected from IL-1, IL-10, and IL-7, TGF-beta receptor, PD-1 or OX40.

In some embodiments, the first fusion protein and the second fusion protein are bound through the first recruitment domain and the second recruitment domain. In some embodiments, the composition further comprises a protease inhibitor bound to the repressible protease.

The present disclosure further provides a composition of an inducible cell receptors comprising two fusion proteins—a. a first fusion protein comprising: (a) an extracellular protein binding domain and (b) a first recruitment domain operably linked to the extracellular protein binding domain, and a repressible protease, and b. a second fusion protein comprising: (a) a first intracellular signaling domain, (b) a second recruitment domain, (c) a cognate cleavage site, and (d) a degradation sequence.

In some embodiments, the cognate cleavage site and the degradation sequence are physically linked to one another. In some embodiments, the cognate cleavage site and the degradation sequence are located at the C-terminus of the second fusion protein. In some embodiments, the repressible protease is located at the C-terminus of the first fusion protein.

In some embodiments, the first fusion protein further comprises a second intracellular signaling domain. In some embodiments, the second fusion protein further comprises a third intracellular signaling domain.

In some embodiments, the second fusion protein further comprises a second extracellular protein binding domain.

In some embodiments, the first fusion protein and the second fusion protein are bound through the first recruitment domain and the second recruitment domain. In some embodiments, the composition further comprises a protease inhibitor bound to the repressible protease.

In yet another aspect, the present disclosure provides a composition of an inducible cell receptor comprising two fusion proteins—a. a first fusion protein comprising: (a) an extracellular protein binding domain (b) a first recruitment domain, and (c) a cognate cleavage site; and b. a second fusion protein comprising: (a) a second recruitment domain, (b) a transmembrane domain, and (c) a repressible protease.

In some embodiments, (a) the first fusion protein is a soluble protein; (b) the first fusion protein is a membrane-bound protein comprising a transmembrane domain, and the first recruitment domain is located between the extracellular protein binding domain and the transmembrane domain; or (c) the first fusion protein is a membrane-bound protein comprising a transmembrane domain, and the transmembrane domain is located between the first recruitment domain and the extracellular protein binding domain.

In some embodiments, (a) the second fusion protein comprises from the N-terminus to the C-terminus the second recruitment domain, the transmembrane domain, and the repressible protease; or (b) the second fusion protein comprises from the N-terminus to the C-terminus the transmembrane domain, the second recruitment domain, and the repressible protease.

In some embodiments, the first fusion protein is a soluble protein and the cognate cleavage site is located between the extracellular protein binding domain and the first recruitment domain.

In some embodiments, the first fusion protein is a membrane-bound protein comprising a transmembrane domain, wherein the first fusion protein further comprises a first intracellular signaling domain, and the cognate cleavage site is located: a. between the extracellular protein binding domain and the transmembrane domain; b. between the transmembrane domain and the first recruitment domain; c. between the transmembrane domain and the first intracellular signaling domain; or d. between the first recruitment domain and the first intracellular signaling domain.

In some embodiments, the second fusion further comprises a second intracellular signaling domain. In some embodiments, the first fusion protein further comprises a second intracellular signaling domain.

In some embodiments, the second fusion protein further comprises a second extracellular protein binding domain.

In some embodiments, the first fusion protein and the second fusion protein are bound through the first recruitment domain and the second recruitment domain. In some embodiments, the composition further comprises a protease inhibitor bound to the repressible protease.

In one aspect, the present disclosure provides a composition of an inducible cell receptor comprising two fusion proteins—a. a first fusion protein comprising: (a) an extracellular protein binding domain (b) a transmembrane domain, (c) first recruitment domain, and (d) a self-excising degron, wherein the degron comprises a repressible protease, a cognate cleavage site, and a degradation sequence; and b. a second fusion protein comprising: (a) a transmembrane domain, (b) a second recruitment domain, and (c) one or more intracellular signaling domains. In some embodiments, the self-excising degron is located at the C-terminus of the first fusion protein.

In some embodiments, the repressible protease is hepatitis C virus (HCV) nonstructural protein 3 (NS3). In some embodiments, the cognate cleavage site comprises an NS3 protease cleavage site. In some embodiments, the NS3 protease cleavage site comprises a NS3/NS4A, a NS4A/NS4B, a NS4B/NS5A, or a NS5A/NS5B junction cleavage site.

In some embodiments, the protease inhibitor is selected from the group consisting of simeprevir, danoprevir, asunaprevir, ciluprevir, boceprevir, sovaprevir, paritaprevir and telaprevir.

In some embodiments, the repressible protease, the cognate cleavage site and the protease inhibitor are those selected from Table 1.

In some embodiments, the degradation sequence is at least 90% identical to the sequence identified by SEQ ID NO: 1. In some embodiments, the degradation sequence comprises the sequence identified by SEQ ID NO: 1.

In some embodiments, the degradation sequence is at least 90% identical to the sequence identified as any one of SEQ ID NOs: 12-20. In some embodiments, the degradation sequence comprises the sequence identified as any one of SEQ ID NOs: 12-20.

In some embodiments, the first intracellular signaling domain comprises CD3zeta, CD28, ZAP40, 4-1BB (CD137), CD28, ICOS, BTLA, OX-40, CD27, CD30, GITR, HVEM, DAP10, DAP12, CD2, MyD88, or a fragment thereof. In some embodiments, the first signaling domain comprises immunoreceptor tyrosine-based activation motif (ITAM).

In some embodiments, the fusion protein comprises a second intracellular signaling domain, wherein the second intracellular signaling domain comprises CD3zeta, CD28, ZAP40, 4-1BB (CD137), CD28, ICOS, BTLA, OX-40, CD27, CD30, GITR, HVEM, DAP10, DAP12, CD2, MyD88, or a fragment thereof. In some embodiments, the fusion protein comprises a second intracellular signaling domain, wherein the second intracellular signaling domain comprises immunoreceptor tyrosine-based activation motif (ITAM).

In some embodiments, the fusion protein comprises a third intracellular signaling domain, wherein the third intracellular signaling domain comprises CD3zeta, CD28, ZAP40, 4-1BB (CD137), CD28, ICOS, BTLA, OX-40, CD27, CD30, GITR, HVEM, DAP10, DAP12, CD2, MyD88, or a fragment thereof. In some embodiments, the fusion protein comprises a third intracellular signaling domain, wherein the third intracellular signaling domain comprises immunoreceptor tyrosine-based activation motif (ITAM).

In some embodiments, the extracellular protein binding domain comprises an antibody, or a fragment thereof. In some embodiments, the extracellular protein binding domain comprises a scFv. In some embodiments, the extracellular protein binding domain comprises a ligand-receptor.

In some embodiments, the first and second recruitment domains are pairs of constitutive protein interaction domains selected from the group consisting of (a) cognate leucine zipper domains, (b) cognate PSD95- Dlgl-zo-1 (PDZ) domains, (c) a streptavidin domain and cognate streptavidin binding protein (SBP) domain, (d) a PYL domain and cognate ABI domain, (e) a pair of cognate zinc finger domains, (f) a pair of cognate SH3 domains, and (g) a peptide and antibody or antigen-binding fragment thereof that specifically binds to the peptide.

In some embodiments, the peptide is selected from the group consisting of: peptide neoepitopes (PNEs), naturally occurring peptides, non-human peptides, yeast peptides, synthetic peptide tags, peptide nucleic acid (PNA), a SunTags, myc-tags, His-tags, HA-tags, peridinin chlorophyll protein complex, green fluorescent protein (GFP), red fluorescent protein (RFP), phycoerythrin (PE), streptavidin, avidin, horse radish peroxidase (HRP), alkaline phosphatase, glucose oxidase, glutathione-S-transferase (GST), maltose binding protein, V5, VSVG, softag 1, softag 3, express tag, S tag, palmitoylation, nitrosylation, SUMO tags, thioredoxin, polyfNANP, poly-Arg, calmodulin binding proteins, PurF fragment, ketosteroid isomerase, PaP3.30, TAF12 histone fold domains, FKBP-tags, SNAP tags, Halo-tags, peptides from RNAse I, small linear hydrophilic peptides, short linear epitopes, and short linear epitope from human nuclear La protein (E5B9).

In some embodiments, the first recruitment domain comprises: FK506 binding protein (FKBP); calcineurin catalytic subunit A (CnA); cyclophilin; FKBP-rapamycin associated protein (FRB); gyrase B (GyrB); dihydrofolate reductase (DHFR); DmrB; PYL; ABI; Cry2; CIP; GAI; GID1; or a fragment thereof. In some embodiments, the second recruitment domain comprises: FK506 binding protein (FKBP); calcineurin catalytic subunit A (CnA); cyclophilin; FKBP-rapamycin associated protein (FRB); gyrase B (GyrB); dihydrofolate reductase (DHFR); DmrB; PYL; ABI; Cry2; CIP; GAI; GID1; or a fragment thereof.

In some embodiments, the first recruitment domain and the second recruitment domain are selected from: (a) FK506 binding protein (FKBP) and FKBP; (b) FKBP and calcineurin catalytic subunit A (CnA); (c) FKBP and cyclophilin; (d) FKBP and FKBP-rapamycin associated protein (FRB); (e) gyrase B (GyrB) and GyrB; (f) dihydrofolate reductase (DHFR) and DHFR; (g) DmrB and DmrB; (h) PYL and ABI; (i) Cry2 and CIP; and (j) GAI and GID1.

The present disclosure further provides a polynucleotide encoding the fusion protein provided herein, and a vector comprising the polynucleotide. The present disclosure further provides a set of polynucleotides comprising a first polynucleotide encoding the first fusion protein and a second polynucleotide encoding the second fusion protein provided herein. A set of vectors comprising a first vector comprising the first polynucleotide, and a second vector comprising the second polynucleotide are also provided.

The present disclosure also provides a cell comprising the fusion protein described herein. The cell can be an immune cell or a cell line derived from an immune cell. The immune cell can be selected from the group consisting of a T cell, a B cell, an NK cell, an NKT cell, an innate lymphoid cell, a mast cell, an eosinophil, a basophils, a macrophage, a neutrophil, a dendritic cell, and any combinations thereof. In some embodiments, the cell is a mesenchymal stem cell.

In one aspect, the present disclosure provides a pharmaceutical composition comprising the fusion protein or the composition comprising multiple fusion proteins, and an excipient.

In another aspect, the present disclosure provides a pharmaceutical composition comprising a cell comprising an inducible cell receptor described herein and an excipient.

The present disclosure further provides a method of regulating activity of a chimeric antigen receptor (CAR), comprising the steps of: a. providing a population of cells comprising the fusion protein or the composition described herein, and b. contacting the population of cells with a protease inhibitor. In some embodiments, at least 80%, at least 85%, at least 90%, at least 95%, or at least 98% of the population of cells is activated in response to a ligand to the extracellular protein binding domain, prior to the contacting step. In some embodiments, at least 75% of the population of cells is inactivated following the contacting step. In some embodiments, less than 25% of the population of cells is activated following the contacting step.

In some embodiments, the step of contacting the population of cells with a protease inhibitor induces the CAR to be degraded. In some embodiments, the step of contacting induces at least 60%, 70%, 80%, 90%, 95%, 96%, 97%, 98%, or 99% of the CAR to be degraded.

In some embodiments, the step of contacting the population of cells with a protease inhibitor prevents degradation of the CAR. In some embodiments, degradation of at least 60%, 70%, 80%, 90%, 95%, 96%, 97%, 98%, or 99% of the CAR is prevented compared to before the step of contacting.

In some embodiments, the method further comprises the step of removing the protease inhibitor from the population of cells. In some embodiments, the method further comprises the step of administering the population of cells to a subject in need of a cell-based therapy.

In one aspect, the present disclosure provides a method of treating a subject in need of a cell-based therapy comprising the step of: administering to the subject a population of cells comprising the fusion protein or the composition comprising the fusion protein described herein. In some embodiments, the population of cells was cultured in the presence of a protease inhibitor capable of inhibiting the repressible protease. In some embodiments, the population of cells was cultured in the absence of a protease inhibitor capable of inhibiting the repressible protease.

In some embodiments, the method further comprises the step of administering to the subject the protease inhibitor capable of inhibiting the repressible protease. In some embodiments, the method further comprises the step of withdrawing the protease inhibitor capable of inhibiting the repressible protease from the subject.

The present disclosure further provides a method of preparing a population of therapeutic cells, comprising the steps of: a. providing a population of cells comprising a polynucleotide or a set of polynucleotides encoding the fusion protein or the composition thereof, and culturing the population of cells, thereby obtaining the population of therapeutic cells.

In some embodiments, the population of therapeutic cells comprises the fusion protein or the composition comprising the fusion protein. The method can further comprise the step of: a. delivering the polynucleotide encoding the fusion protein of the present disclosure to a population of naïve cells, thereby obtaining the population of cells; or b. delivering the set of polynucleotides comprising a first polynucleotide encoding the first fusion protein; and a second polynucleotide encoding the second fusion protein to a population of naïve cells, thereby obtaining the population of cells. In some embodiments, the culturing step is performed in the presence of a protease inhibitor capable of inhibiting the repressible protease. In some embodiments, the culturing step is performed in the absence of a protease inhibitor capable of inhibiting the repressible protease.

In some embodiments, the method further comprises the step of adding an excipient to the population of therapeutic cells.

### BRIEF DESCRIPTION OF THE DRAWINGS

FIG. 1A shows a schematic of 1^{st} generation, 2^{nd} generation, and 3^{r4d} generation chimeric antigen receptors (CARs). FIG. 1B shows a schematic of 2ⁿ generation chimeric antigen receptor conjugated to a self-excising degron in the absence (left) or in the presence of a protease inhibitor (+drug) (right).

FIG. 2 shows a schematic of exemplary CARs modified with a self-excising degron. The self-excising degron can be modified so that, after protease cleavage, the protease is removed from the CAR (left three) or remains with the CAR (right three).

FIG. 3 shows schematics of multipart CARs modified with a self-excising degron, wherein the repressible protease is in cis with the cleavage site (left three) or in trans with the cleavage site (right three).

FIG. 4A shows schematics of single chain and multichain CARs modified with a self-excising repressible protease. FIG. 4B illustrates removal of a functional element of the CAR, i.e., CD3zeta (left) or CD3zeta and co-signaling domains (right) by the protease. In the presence of a protease inhibitor, functional elements of the CAR are preserved to maintain CAR structural integrity.

FIG. 5 shows schematics of multi-chain CARs modified with self-excising degrons or self-excising repressible proteases that function as logic gates.

FIG. 6 shows schematics of CARs modified with self-excising degrons or self-excising repressible proteases in combination with CAR-regulating proteins. For example, CARs can be regulated by linking the CAR domains (e.g., CD3zeta and co-activating domain 41BB, CD3zeta and co-inhibiting domain CTLA4) to antigen presentation on proximal cells. These conditional CAR systems can be combined with self-excising degrons or self-excising repressible proteases to build logic gates having inputs both from the local cell environment and an externally supplied drug. Examples for AND and NOR gates are shown.

FIG. 7 shows schematics of ligands inducibly tethered to their cognate receptors using self-excising repressible proteases in the absence (left) and in the presence (right) of the protease inhibitor (+drug).

FIG. 8 is a graph of the percentage of yellow fluorescent protein (YFP) positive Jurkat cells over a 7-day period following transduction of the cells with a lentiviral vector carrying a self-excising degron fused to a gene encoding YFP. Cells were incubated in the presence of 1 μM or 2 μM Asunaprevir (ASV) protease inhibitor.

FIG. 9 is a graph of the percentage of chimeric antigen receptor (CAR) positive Jurkat cells following transduction of the cells with a lentiviral vector carrying a self-excising degron fused to a gene encoding a MYC tag-containing CAR. Cells were incubated in a medium in the presence of no ASV, 1 μM ASV, or 2 μM ASV.

FIG. 10 is a graph of data demonstrating functional regulation of a CAR switch of the present disclosure and concomitant regulation of Jurkat cell (human T cell) activation by the CAR. Jurkat cells were transduced with a CAR of the present disclosure that includes an anti-Her2 antibody fragment endodomain, a CD3-zeta signaling domain, a CD28 co-stimulating domain, and a C-terminal self-excising degron. In the absence of exposure to the repressible protease inhibitor, ASV, all of the CAR T cells were activated by exposure to recombinant Her2 protein. By contrast, in the presence of ASV, less than 25% of the CAR T cells were activated by exposure to recombinant Her2 protein.

FIG. 11 illustrates three CAR designs—CAR (left), CAR-SMASh (middle), and CAR-SMASh[GGS] (right)—tested in Example 4.

FIG. 12 provides FACS analysis results demonstrating YFP-expression levels on T cells following transduction of lentivirus expressing CAR (left), CAR-SMASh (middle), or CAR-SMASh[GGS] (right) at various titer (GV).

FIG. 13 provides FACS analysis results demonstrating YFP-expression levels on CD3+CD4+ or CD3+CD8+ T cells following transduction of lentivirus expressing CAR (left), CAR-SMASh (middle), or CAR-SMASh[GGS] (right), in the presence or absence of ASV.

FIG. 14 provides FACS analysis results of YFP-expression levels on CAR-SMASh T cells at increasing concentrations of ASV.

FIG. 15A provides time-course YFP-expression on CAR T cells after application of various concentrations of asunaprevir (ASV). FIG. 15B provides time-course YFP-expression on CAR-SMASh T cells after application of various concentrations of asunaprevir (ASV).

FIG. 16A provides time-course YFP-expression on CAR T cells after removal of asunaprevir (ASV). FIG. 16B provides time-course YFP-expression on CAR-SMASh T cells after removal of asunaprevir (ASV).

FIG. 17 provides cytotoxic activity of CAR-SMASh T cells measured by LDH assay, based on effector-to-target ratio (E:T; T cells to target tumor cells) as well as virus titer used to transduce CAR-SMASh expression into T cells.

FIG. 18A illustrates CAR-SMASh expression on CAR-SMASh T cells in the absence (left) and in the presence (right) of ASV. FIG. 18B compares cytotoxic activity of CAR-SMASh T cells with and without ASV, at various effector-to-target ratio (E:T; T cells to target tumor cells).

FIG. 19A compares IFN-gamma production in CAR T cells and CAR-SMASh T cells with and without ASV. FIG. 19B compares IL-1 alpha production in CAR T cells and CAR-SMASh T cells with and without ASV. FIG. 19C compares IL-6 production in CAR T cells and CAR-SMASh T cells with and without ASV.

FIG. 20 provides is a schematic of an exemplary method of preparing cells for a cell-based therapy using the inducible receptor provided herein.

FIG. 21 provides a schematic of another example of a method of preparing cells for a cell-based therapy, implemented with a ‘kill switch’.

The figures depict various embodiments of the present disclosure for purposes of illustration only. One skilled in the art will readily recognize from the following discussion that alternative embodiments of the structures and methods illustrated herein may be employed without departing from the principles of the present disclosure described herein.

### DETAILED DESCRIPTION

Definitions

Unless defined otherwise, all technical and scientific terms used herein have the meaning commonly understood by a person skilled in the art to which the present disclosure belongs. As used herein, the following terms have the meanings ascribed to them below.

The term, “cell receptor” as used herein, refers to a membrane protein that responds specifically to individual extracellular stimuli and generates intracellular signals that give rise to a particular functional responses. Non-limiting examples of these stimuli/signals include soluble factors generated locally (for example, synaptic transmission) or distantly (for example, hormones and growth factors), ligands on the surface of other cells (e.g., an antigen, such as a cancer antigen), or the extracellular matrix itself. Non-limiting examples of cell receptors include G protein coupled receptors, receptor tyrosine kinases, ligand gated ion channels, integrins, cytokine receptors, and chimeric antigen receptors (CARs).

The term, “chimeric antigen receptor” or alternatively a “CAR” as used herein refers to a polypeptide or a set of polypeptides, which when expressed in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule. In some aspects, the set of polypeptides are contiguous with each other. In some embodiments, the CAR further comprises a spacer domain between the extracellular antigen binding domain and the transmembrane domain. In some embodiments, the set of polypeptides include recruitment domains, such as dimerization or multimerization domains, that can couple the polypeptides to one another. In some embodiments, the CAR comprises a chimeric fusion protein comprising an extracellular antigen binding domain, a transmembrane domain and an intracellular signaling domain comprising a functional signaling domain derived from a stimulatory molecule. In one aspect, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In one aspect, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule.

The term, “extracellular protein binding domain” as used herein, refers to a molecular binding domain which is typically an ectodomain of a cell receptor and is located outside the cell, exposed to the extracellular space. Am extracellular protein binding domain can include any molecule (e.g., protein or peptide) capable of binding to another protein or peptide. In some embodiments, an extracellular protein binding domain comprises an antibody, an antigen-binding fragment thereof, F(ab), F(ab′), a single chain variable fragment (scFv), or a single-domain antibody (sdAb). In some embodiments, an extracellular protein binding domain binds to a hormone, a growth factor, a cell-surface ligand (e.g., an antigen, such as a cancer antigen), or the extracellular matrix.

The term, “intracellular signaling domain” as used herein, refers to a functional endodomain of a cell receptor located inside the cell. Following binding of the molecular binding domain to an antigen, for example, the signaling domain transmits a signal (e.g., proliferative/survival signal) to the cell. In some embodiments, the signaling domain is a CD3-zeta protein, which includes three immunoreceptor tyrosine-based activation motifs (ITAMs). Other examples of signaling domains include CD28, 4-1BB, and OX40. In some embodiments, a cell receptor comprises more than one signaling domain, each referred to as a co-signaling domain.

The term, “transmembrane domain” as used herein, refers to a domain that spans a cellular membrane. In some embodiments, a transmembrane domain comprises a hydrophobic alpha helix. Different transmembrane domains result in different receptor stability. In some embodiments, a transmembrane domain of a cell receptor of the present disclosure comprises a CD3-zeta transmembrane domain or a CD28 transmembrane domain.

The term, “recruitment domain” as used herein, refers to an interaction motif found in various proteins, such as helicases, kinases, mitochondrial proteins, caspases, other cytoplasmic factors, etc. The recruitment domains mediate formation of a large protein complex via direct interactions between recruitment domains. In some embodiments, recruitment domains of the present disclosure are dimerization or multimerization domains.

The term, “cell-based therapy” as used herein, refers to a therapeutic method using cells (e.g., immune cells and/or stem cells) to deliver to a patient (a subject) a gene of interest, such as a therapeutic protein. Cell based-therapies, as provided herein, also encompass preventative and diagnostic regimes. Thus, a gene of interest (and encoded product of interest) used in a cell-based therapy may be a prophylactic molecule (e.g., an antigen intended to induce an immune response) or a detectable molecule (e.g., a fluorescent protein or other visible molecule).

The term, “repressible protease” as used herein, refers to a protease that can be inactivated by the presence or absence of a specific agent (e.g., that binds to the protease). In some embodiments, a repressible protease is active (cleaves a cognate cleavage site) in the absence of the specific agent and is inactive (does not cleave a cognate cleavage site) in the presence of the specific agent. In some embodiments, the specific agent is a protease inhibitor. In some embodiments, the protease inhibitor specifically inhibits a given repressible protease of the present disclosure.

Non-limiting examples of repressible proteases include hepatitis C virus proteases (e.g., NS3 and NS2-3); signal peptidase; proprotein convertases of the subtilisin/kexin family (furin, PCI, PC2, PC4, PACE4, PC5, PC); proprotein convertases cleaving at hydrophobic residues (e.g., Leu, Phe, Val, or Met); proprotein convertases cleaving at small amino acid residues such as Ala or Thr; proopiomelanocortin converting enzyme (PCE); chromaffin granule aspartic protease (CGAP); prohormone thiol protease; carboxypeptidases (e.g., carboxypeptidase E/H, carboxypeptidase D and carboxypeptidase Z); aminopeptidases (e.g., arginine aminopeptidase, lysine aminopeptidase, aminopeptidase B); prolyl endopeptidase; aminopeptidase N; insulin degrading enzyme; calpain; high molecular weight protease; and, caspases 1, 2, 3, 4, 5, 6, 7, 8, and 9. Other proteases include, but are not limited to, aminopeptidase N; puromycin sensitive aminopeptidase; angiotensin converting enzyme; pyroglutamyl peptidase II; dipeptidyl peptidase IV; N-arginine dibasic convertase; endopeptidase 24.15; endopeptidase 24.16; amyloid precursor protein secretases alpha, beta and gamma; angiotensin converting enzyme secretase; TGF alpha secretase; T F alpha secretase; FAS ligand secretase; TNF receptor-I and -II secretases; CD30 secretase; KL1 and KL2 secretases; IL6 receptor secretase; CD43, CD44 secretase; CD 16-1 and CD 16-11 secretases; L-selectin secretase; Folate receptor secretase; MMP 1, 2, 3, 7, 8, 9, 10, 11, 12, 13, 14, and 15; urokinase plasminogen activator; tissue plasminogen activator; plasmin; thrombin; BMP-1 (procollagen C-peptidase); ADAM 1, 2, 3, 4, 5, 6, 7, 8, 9, 10, and 11; and, granzymes A, B, C, D, E, F, G, and H. For a discussion of proteases, see, e.g., V. Y. H. Hook, Proteolytic and cellular mechanisms in prohormone and proprotein processing, R G Landes Company, Austin, Tex., USA (1998); N. M. Hooper et al., Biochem. J. 321 : 265-279 (1997); Z. Werb, Cell 91 : 439-442 (1997); T. G. Wolfsberg et al., J. Cell Biol. 131 : 275-278 (1995); K. Murakami and J. D. Etlinger, Biochem. Biophys. Res. Comm. 146: 1249-1259 (1987); T. Berg et al., Biochem. J. 307: 313-326 (1995); M. J. Smyth and J. A. Trapani, Immunology Today 16: 202-206 (1995); R. V. Talanian et al., J. Biol. Chem. 272: 9677-9682 (1997); and N. A. Thomberry et al., J. Biol. Chem. 272: 17907-17911 (1997), the disclosures of which are incorporated herein.

The term, “cognate cleavage site” as used herein, refers to a specific sequence or sequence motif recognized by and cleaved by the repressible protease. A cleavage site for a protease includes the specific amino acid sequence or motif recognized by the protease during proteolytic cleavage and typically includes the surrounding one to six amino acids on either side of the scissile bond, which bind to the active site of the protease and are used for recognition as a substrate.

The term, “self-excising degron” as used herein, refers to a complex comprising a repressible protease, a cognate cleavage site, and a degradation sequence. A self-excising degron is fused to a gene of interest such that the protease is capable of cleaving the complex containing the gene of interest to separate the degradation sequence from the gene of interest. The protease itself may or may not be removed from the complex containing the gene of interest following cleavage.

The term, “degron” as used herein, refers to a protein or a part thereof that is important in regulation of protein degradation rates. Various degrons known in the art, including but not limited to short amino acid sequences, structural motifs, and exposed amino acids, can be used in various embodiments of the present disclosure. Degrons identified from a variety of organisms can be used.

The term, “degradation sequence” as used herein, refers to a sequence that promotes degradation of an attached protein through either the proteasome or autophagy-lysosome pathways. In preferred embodiments, a degradation sequence is a polypeptide that destabilize a protein such that half-life of the protein is reduced at least two-fold, when fused to the protein. Many different degradation sequences/signals (e.g., of the ubiquitin-proteasome system) are known in the art, any of which may be used as provided herein. A degradation sequence may be operably linked to a cell receptor, but need not be contiguous with it as long as the degradation sequence still functions to direct degradation of the cell receptor. In some embodiments, the degradation sequence induces rapid degradation of the cell receptor. For a discussion of degradation sequences and their function in protein degradation, see, e.g., Kanemaki et al. (2013) Pflugers Arch. 465(3):419-425, Erales et al. (2014) Biochim Biophys Acta 1843(1):216-221, Schrader et al. (2009) Nat. Chem. Biol. 5(11):815-822, Ravid et al. (2008) Nat. Rev. Mol. Cell. Biol. 9(9):679-690, Tasaki et al. (2007)Trends Biochem Sci. 32(11):520-528, Meinnel et al. (2006) Biol. Chem. 387(7):839-851, Kim et al. (2013) Autophagy 9(7): 1100-1103, Varshaysky (2012) Methods Mol. Biol. 832: 1-11, and Fayadat et al. (2003) Mol Biol Cell. 14(3): 1268-1278; herein incorporated by reference

Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

Inducible Cell Receptors

In one aspect, the present disclosure provides an inducible cell receptor, which comprises a fusion protein comprising: (a) an extracellular protein binding domain, and (b) a first intracellular signaling domain, and (c) a transmembrane domain located between the extracellular protein binding domain and the first intracellular signaling domain; and b. a self-excising degron operably linked to the first fusion protein and comprising (a) a repressible protease, (b) a cognate cleavage site, and (c) a degradation sequence.

In another aspect, the present disclosure provides a fusion protein comprising (a) an extracellular protein binding domain, (b) a first intracellular signaling domain, and (c) a transmembrane domain located between the extracellular protein binding domain and the first intracellular signaling domain, (d) a repressible protease, and (e) a cognate cleavage site of the repressible protease.

In yet another aspect, the present disclosure provides a composition comprising multiple fusion proteins—a. a first fusion protein comprising: (a) an extracellular protein binding domain, and (b) a first recruitment domain; and b. a second fusion protein comprising: (a) a second recruitment domain, (b) a transmembrane domain, (c) a first intracellular signaling domain, and a self-excising degron operably linked to the second fusion protein, wherein the self-excising degron comprises (i) a repressible protease, (ii) a cognate cleavage site, and (iii) a degradation sequence.

The present disclosure further provides a composition comprising multiple fusion proteins—a. a first fusion protein comprising: (a) an extracellular protein binding domain, (b) a first recruitment domain, (c) a cognate cleavage site, and (d) a degradation sequence; and b. a second fusion protein comprising: (a) a transmembrane domain, (b) a second recruitment domain, and (c) a repressible protease.

The present disclosure further provides a composition comprising multiple fusion proteins—a. a first fusion protein comprising: (a) an extracellular protein binding domain and (b) a first recruitment domain, and (c) a repressible protease, and b. a second fusion protein comprising: (a) a first intracellular signaling domain, (b) a second recruitment domain, (c) a cognate cleavage site, and (d) a degradation sequence.

The present disclosure further provides a composition comprising multiple fusion proteins—a. a first fusion protein comprising: (a) an extracellular protein binding domain (b) a first recruitment domain, and (c) a cognate cleavage site; and b. a second fusion protein comprising: (a) a second recruitment domain, (b) a transmembrane domain, and (c) a repressible protease.

The present disclosure further provides a composition comprising multiple fusion proteins—a. a first fusion protein comprising: (a) an extracellular protein binding domain (b) a transmembrane domain, (c) first recruitment domain, and (d) a self-excising degron, wherein the degron comprises a repressible protease, a cognate cleavage site, and a degradation sequence; and b. a second fusion protein comprising: (a) a transmembrane domain, (b) a second recruitment domain, and (c) one or more intracellular signaling domains.

On and OFF Switches

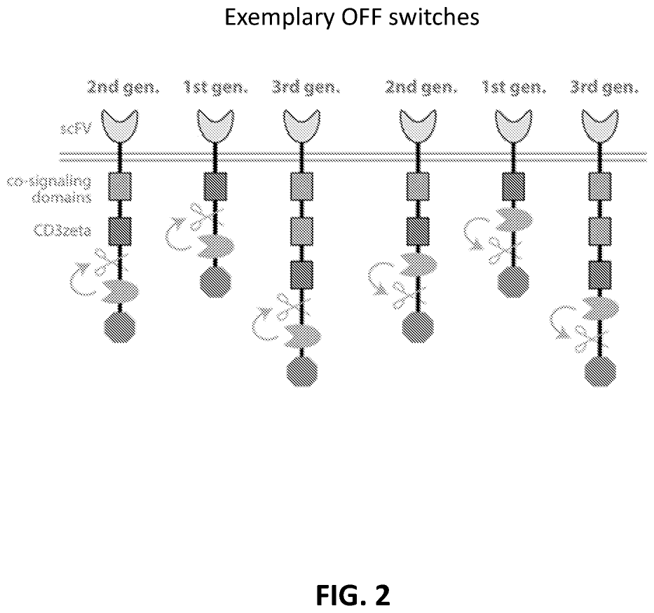

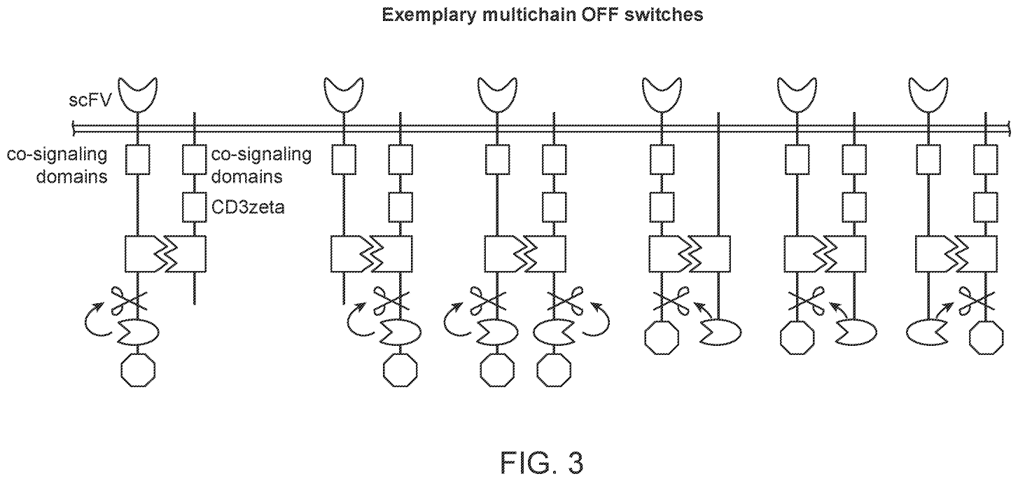

In some embodiments, the present disclosure provides a fusion protein with an “OFF switch,” which is an inducible receptor that is selectively inactivated in the presence of a specific agent. An exemplary OFF switch, as provided herein, may be a cell receptor that comprises (a) a molecular binding domain (e.g., an extracellular protein binding domain), (b) an intracellular signaling domain, (c) a transmembrane domain (e.g., located between the molecular binding domain and the signaling domain), and (d) a self-excising degron that includes a repressible protease, a cognate cleavage site, and a degradation sequence, wherein components (a)-(d) are configured such that the cell receptor is inactivated (does not transmit an intracellular signal) when the repressible protease is repressed. In some embodiments, a self-excising degron is located at the C-terminal (carboxy-terminal) end of product (e.g., protein) encoded by the gene of interest, at the N-terminal (amino-terminal) end of the product, or located within domains of the product (e.g., protein). With OFF switches, cleavage by the repressible protease removes the degradation signal, thereby preserving structural integrity of the receptor, and addition of a specific agent causes degradation of the receptor. See, e.g., FIGS. 2 and 3.

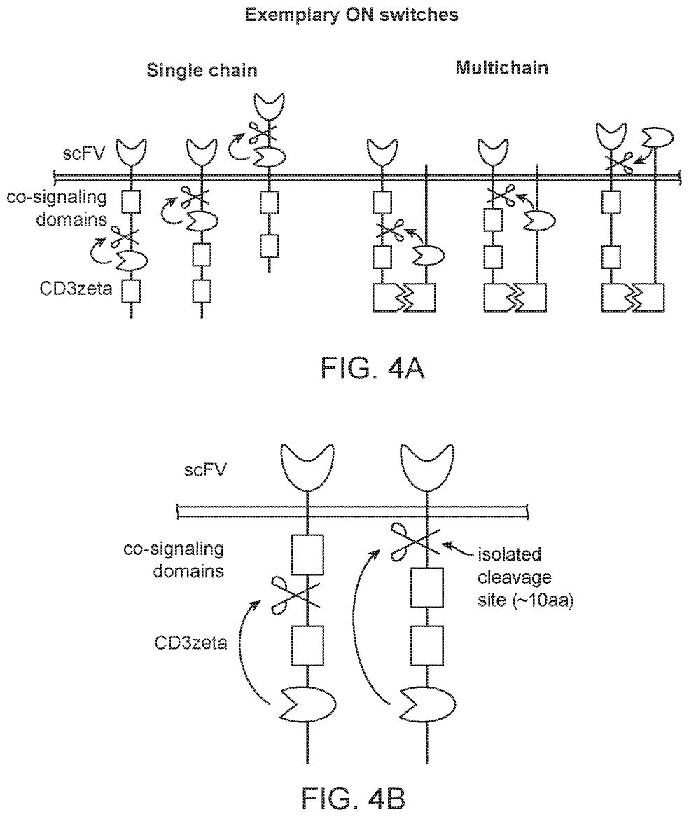

In some embodiments, the present disclosure provides a fusion protein with an “ON switch,” which is an inducible receptor that is selectively activated in the presence of a specific agent. An exemplary ON switch, as provided herein, may be a cell receptor that comprises (a) a molecular binding domain (e.g., an extracellular protein binding domain), (b) a signaling domain, (c) a transmembrane domain (e.g., located between the molecular binding domain and the signaling domain), (d) a repressible protease, and (e) a cognate cleavage site, wherein components (a)-(e) are configured such that the cell receptor is activated (transmits an intracellular signal) when the repressible protease is repressed. Unlike the OFF switches above, the ON switches do not include a degradation sequences. Rather, with ON switches, cleavage by the repressible protease removes a functional element of the cell receptor (e.g., a signaling domain or a protein-binding domain), and addition of a specific agent preserves structural integrity of the receptor. Exemplary ON switches are provided in FIGS. 4A-B.

The repressible protease and the cognate cleavage site of an ON switch may be located between any two domains of a polypeptide of a cell receptor. For example, the repressible protease and the cognate cleavage site may be located between the extracellular protein binding domain and the transmembrane domain. In some embodiments, the repressible protease and the cognate cleavage site are located between the transmembrane domain and the intracellular signaling domain. In other embodiments, repressible protease and the cognate cleavage site are located between two co-signaling domains. In some embodiments, a polynucleotide of a cell receptor further comprises a ligand operably linked to the ligand-binding domain (e.g., an extracellular protein binding domain). In this case, the repressible protease and the cleavage site can be located between the ligand and the ligand-binding domain.

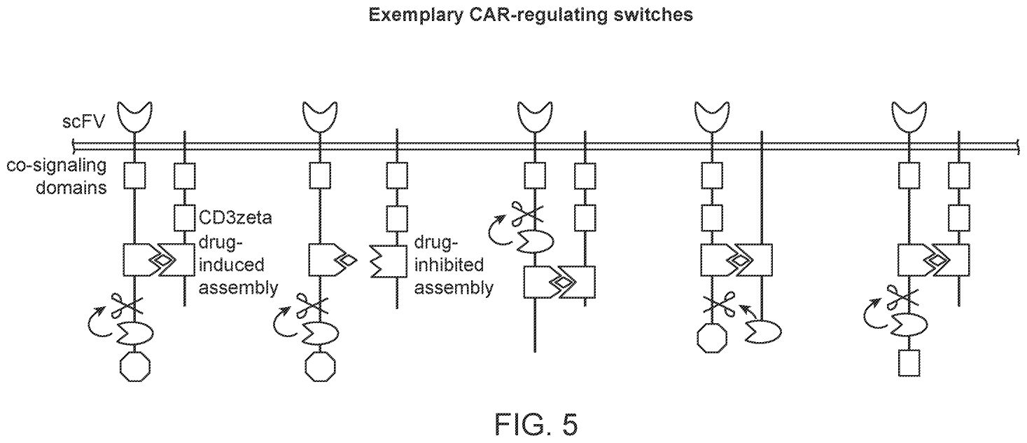

In some embodiments, a cell receptor comprises two polypeptides (e.g., a multichain receptor), as depicted, for example, in FIG. 3 and FIG. 4A. In such embodiments, recruitment domains can be used to bring the two polypeptides together to activate the receptor. Recruitment domains are protein domains that bind to each other and thus, can bring together two different polypeptides, each comprising one of a pair of recruitment domains, as depicted, for example, in FIG. 3 and FIG. 4A. A pair of recruitment domains are considered to assemble with each other if the two domains bind directly to each other, or if the two domains bind to the same (intermediate) molecule, as depicted, for example, in FIG. 5. Non-limiting examples of pairs of recruitment domains include (a) FK506 binding protein (FKBP) and FKBP; (b) FKBP and calcineurin catalytic subunit A (CnA); (c) FKBP and cyclophilin; (d) FKBP and FKBP-rapamycin associated protein (FRB); (e) gyrase B (GyrB) and GyrB; (f) dihydrofolate reductase (DHFR) and DHFR; g) DmrB and DmrB; (g) PYL and ABI; (h) Cry2 and CIP; and (i) GAI and GID1.

In some embodiments of the OFF switches, one polypeptide comprises a protein binding domain, a transmembrane domain, a signaling domain, and a first recruitment domain. In some embodiments, the second polypeptide comprises a second recruitment domain that assembles with the first recruitment domain. In some embodiments, a self-excising degron is located in the first polypeptide or in the second polypeptide. In some embodiments, the components of a self-excising degron are located on different polypeptides that make up a single cell receptor. For example, the repressible protease may be located in one (a first) polypeptide, while the cognate cleavage site and degradation sequence are located in the other (a second) polypeptide.

In some embodiments of the ON switches, a first polypeptide may comprise a protein binding domain, a transmembrane domain, a signaling domain, a first recruitment domain, and a cognate cleavage site. In some embodiments, the second polypeptide comprises the repressible protease and a second recruitment domain that assembles with (binds directly or indirectly to) the first recruitment domain (FIG. 4A).

Also provided herein are methods of regulating activity of a cell receptor (e.g., OFF switches). In some embodiments of the OFF switches, the methods comprise providing a cell comprising cell receptor that includes (a) an extracellular protein binding domain, (b) an intracellular signaling domain, (c) a transmembrane domain located between the protein binding domain and the signaling domain, and (d) a self-excising degron that includes a repressible protease (e.g., NS3 protease), a cognate cleavage site, and a degradation sequence, wherein components (a)-(d) are configured such that the cell receptor is inactivated when the repressible protease is repressed, and contacting the cell with an agent (e.g., simeprevir, danoprevir, asunaprevir, ciluprevir, boceprevir, sovaprevir, paritaprevir, or telaprevir) that represses activity of the repressible protease, thereby inactivating the cell receptor.

In other embodiments of the ON switches, the methods comprise providing a cell comprising a cell receptor that includes (a) an extracellular protein binding domain, (b) an intracellular signaling domain, (c) a transmembrane domain located between the protein binding domain and the signaling domain, (d) a repressible protease (e.g., NS3 protease), and (e) a cognate cleavage site, wherein components (a)-(e) are configured such that the cell receptor is activated when the repressible protease is repressed, and contacting the cell with an agent (e.g., simeprevir, danoprevir, asunaprevir, ciluprevir, boceprevir, sovaprevir, paritaprevir, or telaprevir) that represses activity of the repressible protease, thereby activating the cell receptor.

### Protease, Cognate Cleavage Site, and Protease Inhibitor HCV NS3 Protease Combination

In some embodiments, a hepatitis C virus (HCV) nonstructural protein 3 (NS3) protease is used as a repressible protease. NS3 includes an N-terminal serine protease domain and a C-terminal helicase domain. The protease domain of NS3 forms a heterodimer with the HCV nonstructural protein 4A (NS4A), which activates proteolytic activity. An NS3 protease may comprise the entire NS3 protein or a proteolytically active fragment thereof and may further comprise an activating NS4A region. Advantages of using an NS3 protease include that it is highly selective and can be well-inhibited by a number of non-toxic, cell-permeable drugs, which are currently clinically available.

NS3 protease inhibitors that can be used as provided herein include, but are not limited to, simeprevir, danoprevir, asunaprevir, ciluprevir, boceprevir, sovaprevir, paritaprevir and telaprevir.

When an NS3 protease is used, the cognate cleavage site should comprise an NS3 protease cleavage site. Exemplary NS3 protease cleavage sites include the four junctions between nonstructural (NS) proteins of the HCV polyprotein normally cleaved by the NS3 protease during HCV infection, including the NS3/NS4A, NS4A/NS4B, NS4B/NSSA, and NSSA/NSSB junction cleavage sites. For a description of NS3 protease and representative sequences of its cleavage sites for various strains of HCV, see, e.g., Hepatitis C Viruses: Genomes and Molecular Biology (S.L. Tan ed., Taylor & Francis, 2006), Chapter 6, pp. 163-206; herein incorporated by reference in its entirety. For example, the sequences of HCV NS4A/4B protease cleavage site (SEQ ID NO: 2); HCV NS5A/5B protease cleavage site (SEQ ID NO: 3); C-terminal degradation signal with NS4A/4B protease cleavage site (SEQ ID NO: 4); N-terminal degradation signal with HCV NS5A/5B protease cleavage site (SEQ ID NO: 5) are provided.

NS3 nucleic acid and protein sequences may be derived from HCV, including any isolate of HCV having any genotype (e.g., seven genotypes 1-7) or subtype. A number of NS3 nucleic acid and protein sequences are known. A representative NS3 sequence is presented in SEQ ID NO: 6. Additional representative sequences are listed in the National Center for Biotechnology Information (NCBI) database. See, for example, NCBI entries: Accession Nos. YP_001491553, YP_001469631, YP_001469632, NP_803144, NP_671491, YP_001469634, YP_001469630, YP_001469633, ADA68311, ADA68307, AFP99000, AFP98987, ADA68322, AFP99033, ADA68330, AFP99056, AFP99041, CBF60982, CBF60817, AHH29575, AIZ00747, AIZ00744, ABI36969, ABN05226, KF516075, KF516074, KF516056, AB826684, AB826683, JX171009, JX171008, JX171000, EU847455, EF154714, GU085487, JX171065, JX171063; all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct a cell receptor or a recombinant polynucleotide encoding such a cell receptor, as described herein.

### Other Protease Combinations

Other proteases, including those listed in Table 1, can be used for various embodiments of the present disclosure. When a protease is selected, its cognate cleavage site and protease inhibitors known in the art to bind and inhibit the protease can be used in a combination. Exemplary combinations for the use are provided below in Table 1. Representative sequences of the proteases are available from public database including UniProt through the uniprot.org website. UniProt accession numbers for the proteases are also provided below in Table 1.

TABLE 1

| Protease | | |
|---|---|---|
| (UniProt Accession | | |
| Number or SEQ ID NO.) | Cognate cleavage site | Protease inhibitors |
| HCV NS4A/4B | DEMEECSQHL | Simeprevir, Danoprevir, |
| | (SEQ ID NO: 2) | Asunaprevir, Ciluprevir, |
| | EDVVPCSMG | Boceprevir, Sovaprevir, |
| | (SEQ ID NO: 3) | Paritaprevir, Telaprevir, |
| | | Grazoprevir |
| HCV NS5A/5B | DEMEECSQHL | Simeprevir, Danoprevir, |
| | (SEQ ID NO: 2) | Asunaprevir, Ciluprevir, |
| | EDVVPCSMG | Boceprevir, Sovaprevir, |
| | (SEQ ID NO: 3) | Paritaprevir, Telaprevir, |
| | | Grazoprevir |
| HCV NS3 | DEMEECSQHL | Simeprevir, Danoprevir, |
| | (SEQ ID NO: 2) | Asunaprevir, Ciluprevir, |
| | EDVVPCSMG | Boceprevir, Sovaprevir, |
| | (SEQ ID NO: 3) | Paritaprevir, Telaprevir, |
| | | Grazoprevir |
| HCV NS2-3 | DEMEECSQHL | Simeprevir, Danoprevir, |
| | (SEQ ID NO: 2) | Asunaprevir, Ciluprevir, |
| | EDVVPCSMG | Boceprevir, Sovaprevir, |
| | (SEQ ID NO: 3) | Paritaprevir, Telaprevir, |
| | | Grazoprevir |
| HIV-1 protease | | Amprenavir, Atazanavir, |
| (SEQ ID NO: 7) | | Darunavir, Fosamprenavir, |
| | | Indinavir, Lopinavir, Nelfinavir, |
| | | Ritonavir, Saquinavir, Tipranavir |
| Signal peptidase (P67812, | preference of eukaryotic signal | |
| P15367, P00804, P0803) | peptidase for cleavage after residue 20 | |
| | (Xaa^{20↓}) of pre(Δpro)apoA-II: Ala, Cys > | |
| | Gly > Ser, Thr > Pro > Asn, Val, Ile, | |
| | Leu, Tyr, His, Arg, Asp. | |
| proprotein convertases | (R/K)-X-(hydrophobic)-X↓, where X is | |
| cleaving at hydrophobic | any amino acid | |
| residues (e.g., Leu, Phe, | | |
| Val, or Met) (Q16549, | | |
| Q8NBP7, Q92824, | | |
| P29120, Q6UW60, | | |
| P29122, Q9QXV0) | | |
| proprotein convertases | (K/R)-(X)n-(K/R)↓, where n is 0, 2, 4 or | |
| cleaving at small amino | 6 and X is any amino acid | |
| acid residues such as Ala or | | |
| Thr (Q16549, Q8NBP7, | | |
| Q92824, P29120, | | |
| Q6UW60, P29122) | | |
| proopiomelanocortin | Cleavage at paired basic residues in | |
| converting enzyme (PCE) | certain prohormones, either between | |
| (Q9UO77615, O776133) | them, or on the carboxyl side | |
| chromaffin granule aspartic | tends to cleave dipeptide bonds that | |
| protease (CGAP) | have hydrophobic residues as well as a | |
| | beta-methylene group | |
| prohormone thiol protease | | |
| (cathepsin L1) (P07154, | | |
| P07711, P06797, P25975, | | |
| Q28944) | | |
| carboxypeptidases (e.g., | cleaves a peptide bond at the carboxy- | |
| carboxypeptidase E/H, | terminal (C-terminal) end of a protein or | |
| carboxypeptidase D and | peptide | |
| carboxypeptidase Z) | | |
| (Q9M099, P15169, | | |
| Q04609, P08819, P08818, | | |
| O77564, P70627, O35409, | | |
| P07519, Q8VZU3, P22792, | | |
| P15087, P16870, Q9JHH6, | | |
| Q96IY4, Q7L8A9) | | |
| aminopeptidases (e.g., | cleaves a peptide bond at the amino- | |
| arginine aminopeptidase, | terminal (N-terminal) end of a protein | |
| lysine aminopeptidase, | or peptide | |
| aminopeptidase B) | | |
| prolyl endopeptidase | Hydrolysis of Pro-\|-Xaa >> Ala-\|-Xaa in | |
| (Q12884, P48147, P97321, | oligopeptides. | |
| Q4J6C6) | Release of an N-terminal dipeptide, | |
| | Xaa-Yaa-\|-Zaa-, from a polypeptide, | |
| | preferentially when Yaa is Pro, | |
| | provided Zaa is neither Pro nor | |
| | hydroxyproline | |
| aminopeptidase N (P97449, | Release of an N-terminal amino acid, | |
| P15144, P15145, P15684) | Xaa-\|-Yaa- from a peptide, amide or | |
| | arylamide. Xaa is preferably Ala, but | |
| | may be most amino acids including Pro | |
| | (slow action), When a terminal | |
| | hydrophobic residue is followed by a | |
| | prolyl residue, the two may be released | |
| | as an intact Xaa-Pro dipeptide | |
| insulin degrading enzyme | Degradation of insulin, glucagon and | |
| (P14735, P35559, | other polypeptides. No action on | |
| Q9JHR7, P22817, | proteins. | |
| Q24K02) | Cleaves multiple short polypeptides that | |
| | vary considerably in sequence | |
| Calpain (O08529, P17655, | No specific amino acid sequence is | |
| Q07009, Q27971, P20807, | uniquely recognized by calpains. | |
| P07384, O35350, O14815, | Amongst protein substrates, tertiary | |
| P04632, Q9Y6Q1, | structure elements rather than primary | |
| O15484, Q9HC96, | amino acid sequences appear to be | |
| A6NHC0, Q9UMQ6) | responsible for directing cleavage to a | |
| | specific substrate. Amongst peptide and | |
| | small-molecule substrates, the most | |
| | consistently reported specificity is for | |
| | small, hydrophobic amino acids (e.g., | |
| | leucine, valine and isoleucine) at the P2 | |
| | position, and large hydrophobic amino | |
| | acids (e.g., phenylalanine and tyrosine) | |
| | at the P1 position. One fluorogenic | |
| | calpain substrate is (EDANS)-Glu-Pro- | |
| | Leu-Phe═Ala-Glu-Arg-Lys- | |
| | (DABCYL), | |
| | (EDANSEPLFAERKDABCYL (SEQ | |
| | ID NO: 22)) with cleavage occurring at | |
| | the Phe═Ala bond. | |
| caspase 1 (P29466, | Strict requirement for an Asp residue at | |
| P29452) | position P1 and has a preferred cleavage | |
| | sequence of Tyr-Val-Ala-Asp-\|- | |
| | (YVAD; SEQ ID NO: 23). | |
| caspase 2 (P42575, | Strict requirement for an Asp residue at | |
| P29594) | P1, with 316-asp being essential for | |
| | proteolytic activity and has a preferred | |
| | cleavage sequence of Val-Asp-Val-Ala- | |
| | Asp-\|- (VDVAD; SEQ ID NO: 24). | |
| caspase 3 (P42574, | Strict requirement for an Asp residue at | |
| P70677) | positions P1 and P4. It has a preferred | |
| | cleavage sequence of Asp-Xaa-Xaa- | |
| | Asp-\|- with a hydrophobic amino-acid | |
| | residue at P2 and a hydrophilic amino- | |
| | acid residue at P3, although Val or Ala | |
| | are also accepted at this position. | |
| caspase 4 (P70343, | Strict requirement for Asp at the P1 | |
| P49662) | position. It has a preferred cleavage | |
| | sequence of Tyr-Val-Ala-Asp-\|- | |
| | (YVAD; SEQ ID NO: 23) but also | |
| | cleaves at Asp-Glu-Val-Asp-\|-DEVD; | |
| | SEQ ID NO: 25). | |
| caspase 5 (P51878) | Strict requirement for Asp at the P1 | |
| | position. It has a preferred cleavage | |
| | sequence of Tyr-Val-Ala-Asp-51 - | |
| | (YVAD; SEQ ID NO: 23) but also | |
| | cleaves at Asp-Glu-Val-Asp-\|- -\|- | |
| | (DEVD; SEQ ID NO: 25). | |
| caspase 6 (P55212) | Strict requirement for Asp at position | |
| | P1 and has a preferred cleavage | |
| | sequence of Val-Glu-His-Asp-\|-(VEHD; | |
| | SEQ ID NO: 26). | |
| caspase 7 (P97864, | Strict requirement for an Asp residue at | |
| P55210) | position P1 and has a preferred cleavage | |
| | sequence of Asp-Glu-Val-Asp-51 - | |
| | (DEVD; SEQ ID NO: 25). | |
| caspase 8 (Q8IRY7, | Strict requirement for Asp at position | |
| O89110, Q14790) | P1 and has a preferred cleavage | |
| | sequence of (Leu/Asp/Val)-Glu-Thr- | |
| | Asp-\|-(Gly/Ser/Ala). | |
| caspase 9 (P55211, | Strict requirement for an Asp residue at | |
| Q8C3Q9, Q5IS54) | position P1 and with a marked | |
| | preference for His at position P2. It has | |
| | a preferred cleavage sequence of Leu- | |
| | Gly-His-Asp-\|-Xaa (LGHD; SEQ ID | |
| | NO: 27). | |
| caspase 10 (Q92851) | Strict requirement for Asp at position | |
| | P1 and has a preferred cleavage | |
| | sequence of Leu-Gln-Thr-Asp-\|-Gly | |
| | (LQTDG; SEQ ID NO: 28). | |
| puromycin sensitive | Release of an N-terminal amino acid, | |
| aminopeptidase (P55786, | preferentially alanine, from a wide | |
| Q11011) | range of peptides, amides and | |
| | arylamides. | |
| angiotensin converting | Release of a C-terminal dipeptide, | Benazepril (Lotensin), Captopril, |
| enzyme (ACE) (SEQ ID | oligopeptide-\|-Xaa-Yaa, when Xaa is | Enalapril (Vasotec), Fosinopril, |
| NO: 21) (P12821, P09470, | not Pro, and Yaa is neither Asp nor Glu. | Lisinopril (Prinivil, Zestril), |
| Q9BYF1) | | Moexipril, Perindopril (Aceon), |
| | | Quinapril (Accupril), Ramipril |
| | | (Altace), Trandolapril (Mavik), |
| | | Zofenopril |
| pyroglutamyl peptidase II | Release of the N-terminal pyroglutamyl | |
| (Q9NXJ5) | group from pGlu--His-Xaa tripeptides | |
| | and pGlu--His-Xaa-Gly tetrapeptides | |
| dipeptidyl peptidase IV | Release of an N-terminal dipeptide, | |
| (P27487, P14740, P28843) | Xaa-Yaa-\|-Zaa-, from a polypeptide, | |
| | preferentially when Yaa is Pro, | |
| | provided Zaa is neither Pro nor | |
| | hydroxyproline | |
| N-arginine dibasic | Hydrolysis of polypeptides, preferably | |
| convertase (O43847, | at -Xaa-\|-Arg-Lys-, and less commonly | |
| Q8BHG1) | at -Arg-\|-Arg-Xaa-, in which Xaa is not | |
| | Arg or Lys | |
| endopeptidase 24.15 | Preferential cleavage of bonds with | |
| (thimet oligopeptidase) | hydrophobic residues at Pl, P2 and P3′ | |
| (P52888, P24155) | and a small residue at P1′ in substrates | |
| | of 5 to 15 residues | |
| endopeptidase 24.16 | Preferential cleavage in neurotensin: 10- | |
| (neurolysin) (Q9BYT8, | Pro-\|-Tyr-11 | |
| Q91YP2) | | |
| amyloid precursor protein | Endopeptidase of broad specificity. | |
| secretase alpha (P05067, | | |
| P12023, Q9Y5Z0, P56817) | | |
| amyloid precursor protein | Broad endopeptidase specificity. | |
| secretase beta (P05067, | Cleaves Glu-Val-Asn-Leu-\|-Asp-Ala- | |
| P12023, Q9Y5ZO, P56817) | Glu-Phe (EVNLDAEF; SEQ ID NO: | |
| | 29) in the Swedish variant of | |
| | AlzhFeimer's amyloid precursor protein | |
| amyloid precursor protein | intramembrane cleavage of integral | |
| secretase gamma (P05067, | membrane proteins | |
| P12023, Q9Y5Z0, P56817) | | |
| MMP 1 (P03956, | Cleavage of the triple helix of collagen | SB-3CT |
| Q9EPL5uy) | at about three-quarters of the length of | p-OH SB-3CT |
| | the molecule from the N-terminus, at | O-phosphate SB-3CT |
| | 775-Gly-\|-Ile-776 in the alpha-1(I) | RXP470.1 |
| | chain Cleaves synthetic substrates and | |
| | alpha-macroglobulins at bonds where | |
| | P1′ is a hydrophobic residue. | |
| MMP 2 (P08253, P33434) | Cleavage of gelatin type I and collagen | SB-3CT |
| | types IV, V, VII, X. Cleaves the | p-OH SB-3CT |
| | collagen-like sequence Pro-Gln-Gly-\|- | O-phosphate SB-3CT |
| | Ile-Ala-Gly-Gln (PQGIAGQ; SEQ ID | RXP470.1 |
| | NO: 30). | |
| MMP 3 (P08254, P28862) | Preferential cleavage where P1′, P2′ and | SB-3CT |
| | P3′ are hydrophobic residues. | p-OH SB-3CT |
| | | O-phosphate SB-3CT |
| | | RXP470.1 |
| MMP 7 (P09237, Q10738) | Cleavage of 14-Ala-\|-Leu-15 and 16- | SB-3CT |
| | Tyr-\|-Leu-17 in B chain of insulin. No | p-OH SB-3CT |
| | action on collagen types I, II, IV, V. | O-phosphate SB-3CT |
| | Cleaves gelatin chain alpha-2(I) > | RXP470.1 |
| | alpha-1(I). | |
| MMP 8 (P22894, O70138) | Can degrade fibrillar type I, II, and III | SB-3CT |
| | collagens. | p-OH SB-3CT |
| | Cleavage of interstitial collagens in the | O-phosphate SB-3CT |
| | triple helical domain. Unlike EC | RXP470.1 |
| | 3.4.24.7, this enzyme cleaves type III | |
| | collagen more slowly than type I. | |
| MMP 9 (P14780, P41245) | Cleavage of gelatin types land V and | SB-3CT |
| | collagen types IV and V. | p-OH SB-3CT |
| | Cleaves KiSS1 at a Gly-\|-Leu bond. | O-phosphate SB-3CT |
| | Cleaves type IV and type V collagen | RXP470.1 |
| | into large C-terminal three quarter | |
| | fragments and shorter N-terminal one | |
| | quarter fragments. Degrades fibronectin | |
| | but not laminin or Pz-peptide. | |
| MMP 10 (P09238, | Can degrade fibronectin, gelatins of | SB-3CT |
| O55123) | type I, III, IV, and V; weakly collagens | p-OH SB-3CT |
| | III, IV, and V. | O-phosphate SB-3CT |
| | | RXP470.1 |
| MMP 11 (P24347, | A(A/Q)(N/A),I (L/Y)(T/V/M/R)(R/K) | SB-3CT |
| Q02853) | G(G/A)E↓ILR (SEQ ID NO: 33) | p-OH SB-3CT |
| | ↓ denotes the cleavage site | O-phosphate SB-3CT |
| | | RXP470.1 |
| MMP 12 (P39900, P34960) | Hydrolysis of soluble and insoluble | SB-3CT |
| | elastin. Specific cleavages are also | p-OH SB-3CT |
| | produced at 14-Ala-\|-Leu-15 and 16- | O-phosphate SB-3CT |
| | Tyr--Leu-17 in the B chain of insulin | RXP470.1 |
| | Has significant elastolytic activity. Can | |
| | accept large and small amino acids at | |
| | the P1 site, but has a preference for | |
| | leucine. Aromatic or hydrophobic | |
| | residues are preferred at the P1 site, | |
| | with small hydrophobic residues | |
| | (preferably alanine) occupying P3 | |
| MMP 13 (P45452, P33435) | Cleaves triple helical collagens, | SB-3CT |
| | including type I, type II and type III | p-OH SB-3CT |
| | collagen, but has the highest activity | O-phosphate SB-3CT |
| | with soluble type II collagen. Can also | RXP470.1 |
| | degrade collagen type IV, type XIV and | |
| | type X | |
| MMP 14 (P50281, P53690) | Activates progelatinase A by cleavage | SB-3CT |
| | of the propeptide at 37-Asn-\|-Leu-38. | p-OH SB-3CT |
| | Other bonds hydrolyzed include 35- | O-phosphate SB-3CT |
| | Gly--Ile-36 in the propeptide of | RXP470.1 |
| | collagenase 3, and 341-Asn-\|-Phe-342, | |
| | 441-Asp-\|-Leu-442 and 354-Gln-\|-Thr- | |
| | 355 in the aggrecan interglobular | |
| | domain. | |
| urokinase plasminogen | Specific cleavage of Arg-\|-Val bond in | Plasminogen activator inhibitors |
| activator (uPA) (P00749, | plasminogen to form plasmin. | (PAI) |
| P06869) | | |
| tissue plasminogen | Specific cleavage of Arg-\|-Val bond in | Plasminogen activator inhibitors |
| activator (tPA) (P00750, | plasminogen to form plasmin. | (PAI) |
| P11214) | | |
| tissue plasminogen | Specific cleavage of Arg--Val bond in | Plasminogen activator inhibitors |
| activator (tPA) (P00750, | plasminogen to form plasmin. | (PAI) |
| P11214) | | |
| Plasmin (P00747, P20918) | Preferential cleavage: Lys-\|-Xaa > | α-2-antiplasmin (AP) |
| | Arg-\|-Xaa, higher selectivity than trypsin. | |
| | Converts fibrin into soluble products. | |
| Thrombin (P00734, | Cleaves bonds after Arg and Lys | |
| P19221) | Converts fibrinogen to fibrin and | |
| | activates factors V, VII, VIII, XIII, and, | |
| | in complex with thrombomodulin, | |
| | protein C. | |
| BMP-1 (procollagen C- | Cleavage of the C-terminal propeptide | |
| peptidase) (P13497, | at Ala-\|-Asp in type I and II | |
| P98063) | procollagens and at Arg-\|-Asp in type | |
| | III. | |
| ADAM (Q9P0K1, | | SB-3CT |
| Q9UKQ2, Q9JLN6, | | p-OH SB-3CT |
| 014672, Q13444, P78536, | | O-phosphate SB-3CT |
| Q13443, O43184, P78325, | | RXP470.1 |
| Q9UKF5, Q9BZ11, | | |
| Q9H2U9, Q99965, | | |
| O75077, Q9H013, | | |
| O43506) | | |
| granzyme A (P12544, | Preferential cleavage: -Arg-\|-Xaa-, | |
| P11032) | -Lys-\|-Xaa- >> -Phe-\|-Xaa- in small | |
| | molecule substrates. | |
| granzyme B (P10144, | Preference for bulky and aromatic | |
| P04187) | residues at the P1 position and acidic | |
| | residues at the P3′ and P4′ sites. | |
| granzyme M (P51124, | Cleaves peptide substrates after | |
| Q03238) | methionine, leucine, and norleucine. | |
| tobacco Etch virus (TEV) | E-Xaa-Xaa-Y -Xaa-Q-(G/S), with | |
| protease (P04517, | cleavage occurring between Q and G/S. | |
| POCK09) | The most common sequence is | |
| | ENLYFQS (SEQ ID NO: 31) | |
| chymotrypsin-like serine | | -Thermobilida fusca Thermopin |
| protease (P08217, | | -Pyrobaculum aerophilum Aeropin |
| Q9UNI1, Q91X79, | | -Thermococcus kodakaraensis Tk- |
| P08861, P09093, P08218) | | serpin |
| | | -Alteromonas sp. Marinostatin |
| | | -Streptomyces misionensis SMTI |
| | | -Streptomyces sp. chymostatin |
| alphavirus proteases | | |
| (P08411, P03317, P13886, | | |
| Q8JUX6, Q86924, | | |
| Q4QXJ8, Q8QL53, | | |
| P27282, Q5XXP4) | | |
| chymotrypsin-like cysteine | | -Thermobilida fusca Thermopin |
| proteases (Q86TLO, | | -Pyrobaculum aerophilum Aeropin |
| Q14790, Q99538, O15553) | | -Thermococcus kodakaraensis Tk- |
| | | serpin |
| | | -Alteromonas sp. Marinostatin |
| | | -Streptomyces misionensis SMTI |
| | | -Streptomyces sp. chymostatin |
| papain-like cysteine | | |
| proteases (P25774, | | |
| P53634, Q96K76) | | |
| picornavirus leader | | |
| proteases (P03305, | | |
| P03311, P13899) | | |
| HIV proteases (P04585, | | |
| P03367, P04584, P03369, | | |
| P12497, P03366, P04587) | | |
| Herpesvirus proteases | | |
| (P10220, Q2HRB6, | | |
| O40922, Q69527) | | |
| adenovirus proteases | | |
| (P03252, P24937, Q83906, | | |
| P68985, P09569, P11825, | | |
| P10381) | | |
| Streptomyces griseus | | |
| protease A (SGPA) | | |
| (P00776) | | |
| Streptomyces griseus | | |
| protease B (SGPB) | | |
| (P00777) | | |
| alpha-lytic protease | | |
| (P85142, P00778) | | |
| serine proteases (P48740, | | |
| P98064, Q9UL52, P05981, | | |
| O60235) | | |
| cysteine proteases | | |
| (Q86TL0, Q14790, | | |
| Q8WYN0, Q96DT6, | | |
| P55211) | | |
| aspartic proteases | | |
| (Q9Y5Z0, P56817, | | |
| Q00663, Q53RT3, | | |
| P0CY27) | | |
| threonine proteases | | |
| (Q9UI38, Q16512, | | |
| Q9H6P5, Q8IWU2) | | |
| Mast cell (MC) chymase | Abz-HPFHL-Lys(Dnp)-NH2 (SEQ ID | BAY 1142524 |
| (CMA1) (NM_001836) | NO: 32) | SUN13834 |
| Rat mast cell protease-1, | Abz-HPFHL-Lys(Dnp)-NH2 (SEQ ID | TY-51469 |
| -2, -3, -4, -5 (NM_017145, | NO: 32) | |
| NM_172044, | | |
| NM_001170466, | | |
| NM_019321, NM_013092) | | |
| Rat vascular chymase | Abz-HPFHL-Lys(Dnp)-NH2 (SEQ ID | |
| (RVCH) (O70500) | NO: 32) | |
| DENV NS3pro | A strong preference for basic amino | Anthraquinone |
| (NS2B/N53) | acid residues (Arg/Lys) at the P1 | BP13944 |
| (SEQ ID NO: 8, 9, 10, 11) | positions was observed, whereas the | ZINC04321905 |
| | preferences for the P2-4 sites were in | MB21 |
| | the order of Arg > Thr > Gln/Asn/Lys | Policresulen |
| | for P2, Lys > Arg > Asn for P3, and Nle > | SK-12 |
| | Leu > Lys > Xaa for P4. The prime | NSC135618 |
| | site substrate specificity was for small | Biliverdin |
| | and polar amino acids in P1 and P3. | |

### Degradation Sequences

Degradation sequences known in the art can be used for various embodiments of the present disclosure. In some embodiments, the degradation sequence is at least 80% identical to the sequence identified by SEQ ID NO: 1. In some embodiments, the degradation sequence is at least 85% identical to the sequence identified by SEQ ID NO: 1. In some embodiments, the degradation sequence is at least 90% identical to the sequence identified by SEQ ID NO: 1. In some embodiments, the degradation sequence is at least 95% identical to the sequence identified by SEQ ID NO: 1. In some embodiments, the degradation sequence comprises the sequence identified by SEQ ID NO: 1.

In some embodiments, a degradation sequence comprises a degron identified from an organism, or a modification thereof. Such a degradation sequence includes, but not limited to, HCV NS4 degron, PEST (Two copies of residues 277-307 of IκBα (human); SEQ ID NO: 12), GRR (Residues 352-408 of p105 (human); SEQ ID NO: 13), DRR (Residue 210-295 of Cdc34 (yeast); SEQ ID NO: 14), SNS (Tandem repeat of SP2 and NB (SP2-NB-SP2) (Influenza A and B); SEQ ID NO: 15), RPB (Four copies of residues 1688-1702 of RPB1 (yeast); SEQ ID NO: 16), SPmix (Tandem repeat of SP1 and SP2 (SP2-SP1-SP2-SP1-SP2) (Influenza A virus M2 protein); SEQ ID NO: 17), NS2 (Three copies of residue 79-93 of Influenza A virus NS protein; SEQ ID NO: 18), ODC (Residue 106-142 of ornithine decarboxylase; SEQ ID NO: 19), Nek2A (human), mODC (amino acids 422-461 (mouse); SEQ ID NO: 20), mODC_DA (amino acids 422-461 of mODC (D433A, D434A point mutations (mouse)), APC/C degrons (e.g., D box, KEN box and ABBA motif), COP1 E3 ligase binding degron motif, CRL4-Cdt2 binding PIP degron, Actinfilin-binding degron, KEAP1 binding degron, KLHL2 and KLHL3 binding degron, MDM2 binding motif, N-degron (e.g., Nbox, or UBRbox), Hydroxyproline modification in hypoxia signaling, Phytohormone-dependent SCF-LRR-binding degrons, SCF ubiquitin ligase binding Phosphodegrons, Phytohormone-dependent SCF-LRR-binding degrons, DSGxxS (SEQ ID NO: 34) phospho-dependent degron, Siah binding Motif, SPOP SBC docking motif, PCNA binding PIP box.

In some embodiments, the degradation sequence is at least 80% identical to the sequence identified as any one of SEQ ID NOs: 12-20. In some embodiments, the degradation sequence is at least 85% identical to the sequence identified as any one of SEQ ID NOs: 12-20. In some embodiments, the degradation sequence is at least 90% identical to the sequence identified as any one of SEQ ID NOs: 12-20. In some embodiments, the degradation sequence is at least 95% identical to the sequence identified as any one of SEQ ID NOs: 12-20. In some embodiments, the degradation sequence comprises the sequence identified as any one of SEQ ID NOs: 12-20.

Chimeric Antigen Receptors (CARs)

The cell receptors of the present disclosure, in some embodiments, are chimeric antigen receptors (CARs). CARs, generally, are artificial immune cell receptors engineered to recognize and bind to an antigen expressed by tumor cells. CARs may typically include an antibody fragment as an antigen-binding domain, a spacer domains, a hydrophobic alpha helix transmembrane domain, and one or more intracellular signaling/co-signaling domains, such as (but not limited to) CD3-zeta, CD28, 4-1BB and/or OX40. A CAR can include a signaling domain or at least two co-signaling domains. In some embodiments, a CAR includes three or four co-signaling domains. In some embodiments, a self-excising degron is located in the C-terminus of the CAR. See, e.g., FIGS. 2 and 3.

Generally, a CAR is designed for a T cell, or NK cell, and is a chimera of a signaling domain of the T-cell receptor (TCR) complex and an antigen-recognizing domain (e.g., a single chain fragment (scFv) of an antibody) (Enblad et al., Human Gene Therapy. 2015; 26(8):498-505). A T cell that expresses a CAR is known in the art as a CART cell.

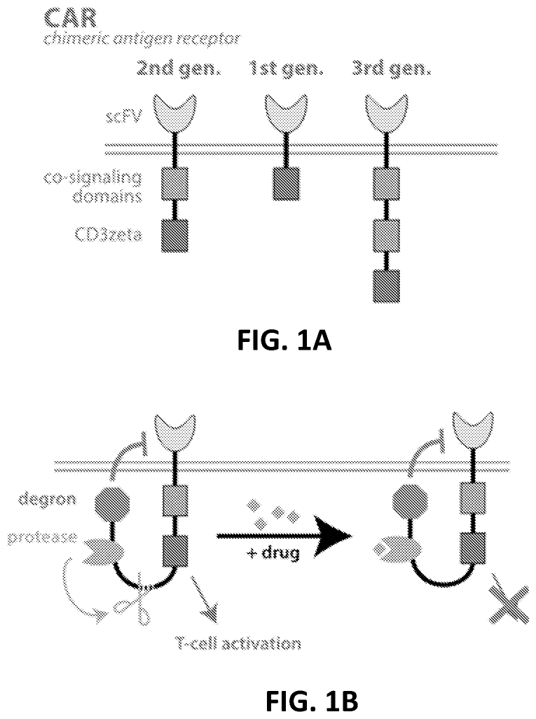

There are at least four generations of CARs, each of which contains different components (FIG. 1A). First generation CARs join an antibody-derived scFv to the CD3zeta ζ or z) intracellular signaling domain of the T-cell receptor through hinge and transmembrane domains. Second generation CARs incorporate an additional domain, e.g., CD28, 4-1BB (41BB), or ICOS, to supply a costimulatory signal. Third-generation CARs contain two costimulatory domains fused with the TcR CD3-ζ chain. Third-generation costimulatory domains may include, e.g., a combination of CD3z, CD27, CD28, 4-1BB, ICOS, or OX40. CARs, in some embodiments, contain an ectodomain (e.g., CD3), commonly derived from a single chain variable fragment (scFv), a hinge, a transmembrane domain, and an endodomain with one (first generation), two (second generation), or three (third generation) signaling domains derived from CD3Z and/or co-stimulatory molecules (Maude et al., Blood. 2015; 125(26):4017-4023; Kakarla and Gottschalk, Cancer J. 2014; 20(2): 151-155).

In some embodiments, a chimeric antigen receptor (CAR) is a T-cell redirected for universal cytokine killing (TRUCK), also known as a fourth generation CAR. TRUCKs are CAR-redirected T-cells used as vehicles to produce and release a transgenic cytokine that accumulates in the targeted tissue, e.g., a targeted tumor tissue. The transgenic cytokine is released upon CAR engagement of the target. TRUCK cells may deposit a variety of therapeutic cytokines in the target. This may result in therapeutic concentrations at the targeted site and avoid systemic toxicity.

CARs typically differ in their functional properties. The CD3ζ signaling domain of the T-cell receptor, when engaged, will activate and induce proliferation of T-cells but can lead to anergy (a lack of reaction by the body's defense mechanisms, resulting in direct induction of peripheral lymphocyte tolerance). Lymphocytes are considered anergic when they fail to respond to a specific antigen. The addition of a costimulatory domain in second-generation CARs improved replicative capacity and persistence of modified T-cells. Similar antitumor effects are observed in vitro with CD28 or 4-1BB CARs, but preclinical in vivo studies suggest that 4-1BB CARs may produce superior proliferation and/or persistence. Clinical trials suggest that both of these second-generation CARs are capable of inducing substantial T-cell proliferation in vivo, but CARs containing the 4-1BB costimulatory domain appear to persist longer. Third generation CARs combine multiple signaling domains (costimulatory) to augment potency. Fourth generation CARs are additionally modified with a constitutive or inducible expression cassette for a transgenic cytokine, which is released by the CAR T-cell to modulate the T-cell response. See, for example, Enblad et al., Human Gene Therapy. 2015; 26(8):498-505; Chmielewski and Hinrich, Expert Opinion on Biological Therapy. 2015;15(8): 1145-1154.

In some embodiments, a chimeric antigen receptor of the present disclosure is a first generation CAR. In some embodiments, a chimeric antigen receptor of the present disclosure is a second generation CAR. In some embodiments, a chimeric antigen receptor of the present disclosure is a third generation CAR. In some embodiments, a chimeric antigen receptor of the present disclosure is a fourth generation CAR.

In some embodiments, a spacer domain or a hinge domain is located between an extracellular domain (e.g., comprising the antigen binding domain) and a transmembrane domain of a CAR, or between a cytoplasmic signaling domain and a transmembrane domain of the CAR. A spacer domain is any oligopeptide or polypeptide that functions to link the transmembrane domain to the extracellular domain and/or the cytoplasmic signaling domain in the polypeptide chain. A hinge domain is any oligopeptide or polypeptide that functions to provide flexibility to the CAR, or domains thereof, or to prevent steric hindrance of the CAR, or domains thereof. In some embodiments, a spacer domain or hinge domain may comprise up to 300 amino acids (e.g., 10 to 100 amino acids, or 5 to 20 amino acids). In some embodiments, one or more spacer domain(s) may be included in other regions of a CAR.

In some embodiments, a CAR is an antigen-specific inhibitory CAR (iCAR), which may be used, for example, to avoid off-tumor toxicity (Fedorov, VD et al. Sci. Transl. Med. 2013, incorporated herein by reference). iCARs contain an antigen-specific inhibitory receptor, for example, to block nonspecific immunosuppression, which may result from extra-tumor target expression. iCARs may be based, for example, on inhibitory molecules CTLA-4 or PD-1. In some embodiments, these iCARs block T cell responses from T cells activated by either their endogenous T cell receptor or an activating CAR. In some embodiments, this inhibiting effect is temporary.

In some embodiments, CARs may be used in adoptive cell transfer, wherein immune cells are removed from a subject and modified so that they express receptors specific to an antigen, e.g., a tumor-specific antigen. The modified immune cells, which may then recognize and kill the cancer cells, are reintroduced into the subject (Pule, et al., Cytotherapy. 2003; 5(3): 211-226; Maude et al., Blood. 2015; 125(26): 4017-4023, each of which is incorporated herein by reference).

### Multipart CARs

The present disclosure provides single chain (polypeptide) cell receptors as well as multichain (and thus multipart) receptors. Thus, an ON switch or an OFF switch may comprise a single polypeptide, or at least two polypeptides.

In some embodiments of an OFF switch, a CAR is a multipart receptor comprising at least two polypeptides. In some embodiments, the CAR comprises a first polypeptide comprising (a) an extracellular protein binding domain (e.g., an antibody fragment), (b) a signaling domain, (c) a transmembrane domain located between the extracellular protein binding domain and the signaling domain, and (d) a first recruitment domain, and a second polypeptide comprising a signaling domain and a second recruitment domain that assembles with the first recruitment domain, wherein a self-excising degron is located in the first polypeptide and/or the second polypeptide. See, e.g., FIG. 3, left schematics. In some embodiments, the self-excising degron is located in the C-terminus of the first polypeptide and/or the second polypeptide.

In other embodiments of an OFF switch, the CAR comprises a first polypeptide comprising (a) an extracellular protein binding domain (e.g., an antibody fragment), (b) a signaling domain, (c) a transmembrane domain located between the an extracellular protein binding domain and the signaling domain, and (d) a first recruitment domain; and a second polypeptide comprising a second recruitment domain that assembles with the first recruitment domain, wherein the repressible protease is located in the first polypeptide, and the cognate cleavage site and degradation sequence are located in the second polypeptide, or wherein the repressible protease is located in the second polypeptide, and the cognate cleavage site and degradation sequence are located in the first polypeptide. See, e.g., FIG. 3, right schematics. In some embodiments, the degradation sequence is located in the C-terminus of the first polypeptide and/or the second polypeptide.

In some embodiments of an ON switch, a CAR comprises a first polypeptide comprising (a) an extracellular protein binding domain (e.g., an antibody fragment), (b) a first intracellular signaling domain, (c) a transmembrane domain located between the antibody fragment and the intracellular signaling domain, (d) a second intracellular signaling domain, and (d) a first recruitment domain; and a second polypeptide comprising the repressible protease and a second recruitment domain that assembles with the first recruitment domain, wherein the cognate cleavage site is located between the antibody fragment and the transmembrane domain, between the transmembrane domain and first intracellular signaling domain, or between the first intracellular signaling domain and the second intracellular signaling domain. See, e.g., FIG. 4A, left schematics.

In other embodiments of an ON switch, a CAR comprises a first polypeptide comprising (a) an extracellular protein binding domain (e.g., an antibody fragment), (b) a first intracellular signaling domain, (c) a transmembrane domain located between the antibody fragment and the intracellular signaling domain, (d) a second intracellular signaling domain, and (d) a first recruitment domain; and a second polypeptide comprising the repressible protease and a second recruitment domain that assembles with the first recruitment domain, wherein the cognate cleavage site is located between the antibody fragment and the transmembrane domain, between the transmembrane domain and first intracellular signaling domain, or between the first intracellular signaling domain and the second intracellular signaling domain. See, e.g., FIG. 4A, right schematics.

### Additional CAR-Regulating Switches

In some embodiments, a self-excising degron (e.g., OFF switch) and/or a repressible protease/cognate cleavage site (e.g., ON switch) may be combined with orthogonal CAR-regulating switches to yield logic gates with, for example, at least 2 agent (e.g., drug) inputs that perform higher order functionalities. Examples for AND, OR, NOR, and conditional ON gates are shown here in FIG. 5. Additional logic gate arrangements for similar CAR gating are encompassed by the present disclosure.

In some embodiments, a CAR comprises a first polypeptide comprising (a) an extracellular protein binding domain (e.g., an antibody fragment), (b) a signaling domain, (c) a transmembrane domain located between the extracellular protein binding domain and the signaling domain, (d) a first recruitment domain, and (e) a self-excising degron that includes a repressible protease, a cognate cleavage site, and a degradation sequence, and a second polypeptide comprising a signaling domain and a second recruitment domain that assembles with the first recruitment domain only when the CAR is contacted with an agent required for assembly of the first recruitment domain with the second recruitment domain. See, e.g., FIG. 5, first schematic. In some embodiments, methods of regulating activity of the CAR comprise contacting a cell comprising the CAR with (a) an agent that represses activity of the repressible protease and (b) an agent required for assembly of the first recruitment domain with the second recruitment domain, thereby activating the CAR.

In other embodiments, a CAR comprises a first polypeptide comprising (a) an extracellular protein binding domain (e.g., an antibody fragment), (b) a signaling domain, (c) a transmembrane domain located between the antibody fragment and the signaling domain, (d) a first recruitment domain, and (e) a self-excising degron that includes a repressible protease, a cognate cleavage site, and a degradation sequence, and a second polypeptide comprising a signaling domain and a second recruitment domain that assembles with the first recruitment domain unless in the CAR is contacted with an agent that prevents assembly of the first recruitment domain with the second recruitment domain. See, e.g., FIG. 5, second schematic. In some embodiments, methods of regulating activity of the CAR comprise contacting a cell comprising the CAR with (a) an agent that represses activity of the repressible protease and (b) an agent that prevents assembly of the first recruitment domain with the second recruitment domain, thereby inactivating the CAR.

In yet other embodiments, a CAR comprises a first polypeptide comprising (a) an antibody fragment, (b) a signaling domain, (c) a transmembrane domain located between the antibody fragment and the signaling domain, (d) a first recruitment domain, and (e) a repressible protease and a cognate cleavage site, wherein the repressible protease and a cognate cleavage site are located between the signaling domain and the first recruitment domain, and a second polypeptide comprising a signaling domain and a second recruitment domain that assembles with the first recruitment domain only when the CAR is contacted with an agent required for assembly of the first recruitment domain with the second recruitment domain. See, e.g., FIG. 5, third schematic. In some embodiments, methods of regulating activity of the CAR comprise contacting a cell comprising the CAR with (a) an agent that represses activity of the repressible protease and (b) an agent required for assembly of the first recruitment domain with the second recruitment domain, thereby activating the CAR.

In still other embodiments, a CAR comprises a first polypeptide comprising (a) an antibody fragment, (b) a signaling domain, (c) a transmembrane domain located between the antibody fragment and the signaling domain, and (d) a first recruitment domain, and a second polypeptide comprising a second recruitment domain that assembles with the first recruitment domain only when the CAR is contacted with an agent required for assembly of the first recruitment domain with the second recruitment domain, wherein the CAR further comprises a self-excising degron comprising a repressible protease, a cognate cleavage site, and a degradation sequence, and wherein the cognate cleavage site and degradation sequence are located at the C-terminus of the first polypeptide and the repressible protease is located at the C-terminus of the second polypeptide. See, e.g., FIG. 5, fourth schematic. In some embodiments, methods of regulating activity of the CAR comprise contacting a cell comprising the CAR with an agent required for assembly of the first recruitment domain with the second recruitment domain, thereby activating the CAR. The methods may further comprise contacting the cell with an agent that represses activity of the repressible protease, thereby inactivating the CAR.

In some embodiments, a CAR comprises a first polypeptide comprising (a) an antibody fragment, (b) a signaling domain, (c) a transmembrane domain located between the antibody fragment and the signaling domain, (d) a first recruitment domain, (e) an inhibitory domain, and (f) a repressible protease and cognate cleavage site located between the first recruitment domain and the inhibitory domain, and a second polypeptide comprising a second recruitment domain that assembles with the first recruitment domain only when the CAR is contacted with an agent required for assembly of the first recruitment domain with the second recruitment domain. See, e.g., FIG. 5, fifth schematic. In some embodiments, methods of regulating activity of the CAR comprise contacting a cell comprising the CAR with an agent required for assembly of the first recruitment domain with the second recruitment domain, thereby activating the CAR. The methods may further comprise contacting the cell with an agent required for assembly of the first recruitment domain with the second recruitment domain, thereby activating the CAR. The methods may further comprise contacting the cell with an agent that represses activity of the repressible protease, thereby inactivating the CAR.

Also provided herein are cells comprising any of the additional CAR-regulating switches described above.

### CAR-Regulating Proteins

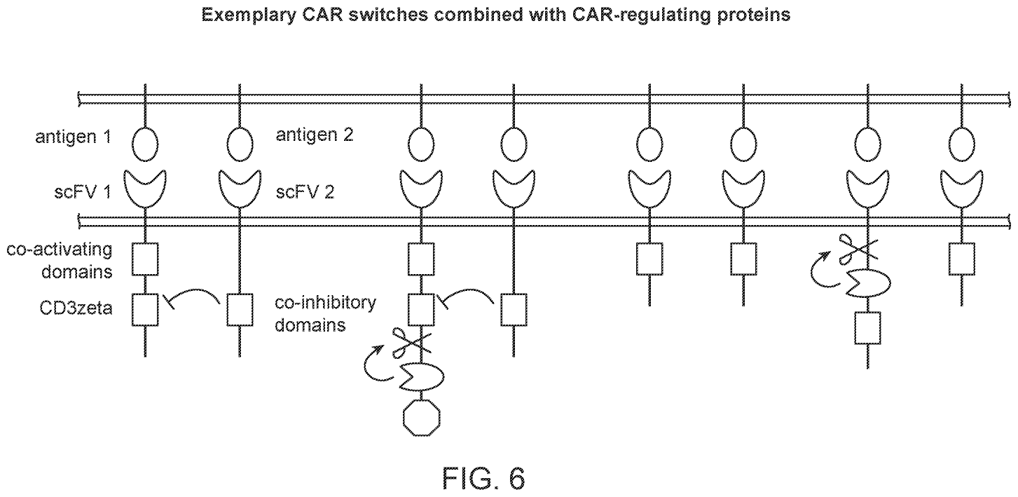

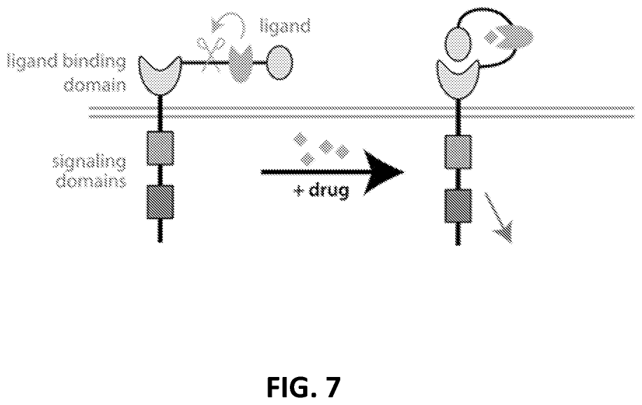

In some embodiments, CARs can be regulated by linking the CAR domains (e.g., CD3zeta and co-activating domain 41BB, CD3zeta and co-inhibiting domain CTLA4) to antigen presentation on proximal cells (FIG. 6). These conditional CAR systems can be combined with a self-excising degron (e.g., OFF switch) and/or a repressible protease/cognate cleavage site (e.g., ON Switch) to build logic gates with inputs that are from both the local cell environment and an externally supplied agent (e.g., drug). Examples for AND and NOR gates are shown here in FIG. 6. Additional logic gate arrangement for similar CAR gating are encompassed by the present disclosure.

In some embodiments, a cell comprises a first polypeptide comprising (a) a first extracellular protein binding domain, (b) an intracellular signaling domain, (c) a transmembrane domain located between the first protein binding domain and the signaling domain, and (d) a self-excising degron comprising a repressible protease, a cognate cleavage site, and a degradation sequence, wherein the self-excising degron is located in the C-terminus of the first polypeptide, and a second polypeptide comprising (a) a second extracellular protein binding domain, (b) an intracellular inhibitory domain that inhibits signaling of the signaling domain of the first polypeptide, and (c) a transmembrane domain located between the second protein binding domain and the inhibitory domain. See, e.g., FIG. 6, left schematics.

In other embodiments, a cell comprises first polypeptide comprising (a) a first extracellular protein binding domain, (b) a first intracellular signaling domain, (c) a transmembrane domain located between the first protein binding domain and the first signaling domain, and (d) a repressible protease and cognate cleavage site located between the transmembrane domain and the first signaling domain, and a second polypeptide comprising (a) a second extracellular protein binding domain, (b) a second intracellular signaling domain, and (c) a transmembrane domain located between the second protein binding domain and the second signaling domain. See, e.g., FIG. 6, right schematics.

Also provided herein are cells comprising any of the CAR-regulating proteins described above.

### Elements of CARs

CARs typically include an extracellular protein binding domain (e.g., antibody fragment as an antigen-binding domain), a spacer domain, a transmembrane domain, and one or more intracellular signaling/co-signaling domains. In some embodiments, CARs of the present disclosure may also include a recruitment domain.

### Extracellular Protein Binding Domain

In some embodiments, an extracellular protein binding domain of a CAR of the disclosure comprises an antigen binding domain, such as a single chain Fv (scFv) specific for a tumor antigen. In some embodiments, an extracellular protein binding domain comprises an antibody, an antigen-binding fragment thereof, F(ab), F(ab′), a single chain variable fragment (scFv), or a single-domain antibody (sdAb).

In some embodiments, the extracellular protein binding domain comprises a ligand-binding domain. The ligand-binding domain can be a domain from a receptor, wherein the receptor is selected from the group consisting of TCR, BCR, a cytokine receptor, RTK receptors, serine/threonine kinase receptors, hormone receptors, immunoglobulin superfamily receptors, and TNFR-superfamily of receptors. In some embodiments, the receptor is a cytokine receptor selected from IL-1, IL-10, and IL-7, TGF-beta receptor, PD-1 or OX40.

The choice of binding domain depends upon the type and number of ligands that define the surface of a target cell. For example, the antigen binding domain may be chosen to recognize a ligand that acts as a cell surface marker on target cells associated with a particular disease state, such as cancer or an autoimmune disease. Thus, examples of cell surface markers that may act as ligands for the antigen binding domain in the CAR of the present disclosure include those associated with cancer cells and/or other forms of diseased cells. In some embodiments, a CAR is engineered to target a tumor antigen of interest by way of engineering a desired antigen binding domain that specifically binds to an antigen on a tumor cell encoded by an engineered nucleic acid.

An antigen binding domain (e.g., an scFv) that specifically binds to a target or an epitope is a term understood in the art, and methods to determine such specific binding are also known in the art. A molecule is said to exhibit specific binding if it reacts or associates more frequently, more rapidly, with greater duration and/or with greater affinity with a particular target antigen than it does with alternative targets. An antigen binding domain (e.g., an scFv) that specifically binds to a first target antigen may or may not specifically bind to a second target antigen. As such, specific binding does not necessarily require (although it can include) exclusive binding.

In some embodiments, immune cells expressing a CAR are genetically modified to recognize multiple targets or antigens, which permits the recognition of unique target or antigen expression patterns on tumor cells. Examples of CARs that can bind multiple targets include: “split signal CARs,” which limit complete immune cell activation to tumors expressing multiple antigens; “tandem CARs” (TanCARs), which contain ectodomains having two scFvs; and “universal ectodomain CARs,” which incorporate avidin or a fluorescein isothiocyanate (FITC)-specific scFv to recognize tumor cells that have been incubated with tagged monoclonal antibodies (Mabs).

A CAR is considered “bispecific” if it recognizes two distinct antigens (has two distinct antigen recognition domains). In some embodiments, a bispecific CAR is comprised of two distinct antigen recognition domains present in tandem on a single transgenic receptor (referred to as a TanCAR; see, e.g., Grada Z et al. Molecular Therapy Nucleic Acids 2013;2:e105, incorporated herein by reference).

### Intracellular Signaling Domain

In some embodiments, the fusion protein comprises one or more intracellular signaling domains. An intracellular signaling domain that is of particular use in the present disclosure includes, but is not limited to, those derived from CD3 zeta, common FcR gamma (FCER1G), Fc gamma Rlla, FcR beta (Fc epsilon lb), CD3 gamma, CD3 delta, CD3 epsilon, CD3, CD22, CD79a, CD79b, CD278 (also known as “ICOS”), FcsRI, DAP10, DAP12, and CD66d.

In some embodiments, an intracellular signaling domain is derived from a signaling region of 4-1BB/CD137, activating NK cell receptors, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD11a, CD11b, CD11c, CD11d, CDS, CEACAM1, CRT AM, cytokine receptors, DAP-10, DNAM1 (CD226), Fc gamma receptor, GADS, GITR, HVEM (LIGHTR), IA4, ICAM-1, ICAM-1, Ig alpha (CD79a), IL2R beta, IL2R gamma, IL7R alpha, Immunoglobulin-like proteins, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, a ligand that specifically binds with CD83, LIGHT, LIGHT (tumor necrosis factor superfamily member 14; TNFSF14), LTBR, Ly9 (CD229), lymphocyte function-associated antigen-1 (LFA-1 (CD11a/CD18), MHC class I molecule, NKG2C, NKG2D, NKp30, NKp44, NKp46, NKp80 (KLRF1), OX-40, PAG/Cbp, programmed death-1 (PD-1), PSGL1, SELPLG (CD162), signaling lymphocytic activation molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, a Toll ligand receptor, TRANCE/RANKL, VLA1, or VLA-6, or a combination thereof.

In some embodiments, an intracellular signaling domain is derived from a signaling region of a protein selected from the group consisting of a MHC class I molecule, a TNF receptor protein, an Immunoglobulin-like protein, a cytokine receptor, an integrm, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD 11 a/CD 18), 4-1BB (CD137), B7-H3, B7-H6, CD3, CD8, CDS, ICAM-1 , ICOS (CD278), GITR, BAFFR, LIGHT, HVEM (LIGHTR), KIRDS2, SLAMF7, NKp80 (KLRF1), NKp44, NKp30, NKp46, CD 19, CD4, CDSalpha, CDSbeta, IL2R beta, IL2R gamma, IL7R alpha, ITGA4, VLA1, CD49a, ITGA4, IA4, CD49D, ITGA6, VLA-6, CD49f, ITGAD, CD 1 id, ITGAE, CD 103, ITGAL, CD 11a, LFA-1, ITGAM, CD 1 ib, ITGAX, CD 11c, ITGBl, CD29, ITGB2, CD 18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD 160 (BY55), PSGL1, CD 100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD 19a, a ligand that specifically binds with CD83, CD70, CD3OL, Cytokine, IL-2, IL-21, CD80, and CD86.

### Transmembrane Domain

The fusion protein further includes a transmembrane domain. A transmembrane domain can be derived from a protein selected from the group consisting of the alpha, beta or zeta chain of the T-cell receptor, CD28, CD3 epsilon, CD45, CD4, CD5, CD8, CD9, CD16, CD22, CD33, CD37, CD4, CD80, CD86, CD134, CD137, CD154, 4-1BB/CD137, an alpha chain of a T cell receptor, a beta chain of a T cell receptor, CD3 epsilon, CD8 alpha, CD19, CD45, CD64, and a zeta chain of a T cell receptor.

### Recruitment Domains

In some embodiments, the fusion protein further includes a recruitment domain, particularly when the CARs are multipart (e.g., split) CARs and thus protein interactions may be important. In such cases, a first protein of the multipart CAR comprises a first recruitment domain and a second protein of the multipart CAR comprises a second recruitment domain. In some embodiments, the first protein of the multipart CAR is a soluble protein that comprises a first recruitment domain and an extracellular protein binding domain, and the second protein is a universal CAR that comprises, as the extracellular protein binding domain, a second recruitment domain that specifically recognizes the first recruitment domain on the first protein. The first and second recruitment domains can be pairs of constitutive protein interaction domains selected from the group consisting of (a) cognate leucine zipper domains, (b) cognate PSD95- Dlgl-zo-1 (PDZ) domains, (c) a streptavidin domain and cognate streptavidin binding protein (SBP) domain, (d) a PYL domain and cognate ABI domain, (e) a pair of cognate zinc finger domains, (f) a pair of cognate SH3 domains, and (g) a peptide and antibody or antigen-binding fragment thereof that specifically binds to the peptide.

When a peptide and antibody or antigen-binding fragment thereof that specifically binds to the peptide is used, the peptide can be peptide neoepitopes (PNEs), naturally occurring peptides, non-human peptides, yeast peptides (e.g., peptides derived from yeast transcription factor GCN4), synthetic peptide tags, peptide nucleic acid (PNA), a SunTags, myc-tags, His-tags, HA-tags, peridinin chlorophyll protein complex, green fluorescent protein (GFP), red fluorescent protein (RFP), phycoerythrin (PE), streptavidin, avidin, horse radish peroxidase (HRP), alkaline phosphatase, glucose oxidase, glutathione-S-transferase (GST), maltose binding protein, V5, VSVG, softag 1, softag 3, express tag, S tag, palmitoylation, nitrosylation, SUMO tags, thioredoxin, polyfNANP, poly-Arg, calmodulin binding proteins, PurF fragment, ketosteroid isomerase, PaP3.30, TAF12 histone fold domains, FKBP-tags, SNAP tags, Halo-tags, peptides from RNAse I, small linear hydrophilic peptides, short linear epitopes, or short linear epitope from human nuclear La protein (E5B9).

In some embodiments, a leucine zipper domain is used as a recruitment domain. A number of leucine zipper domains, as well as their ability to bind each other, are known in the art and discussed further, e.g., in Reinke et al. JACS 2010 132:6025-31 and Thomposon et al. ACS Synth Biol 2012 1 : 118-129; each of which is incorporated by reference herein in its entirety. In some embodiments, two leucine zipper domains are used to induce formation of a complex, where a first recruitment domain is BZip (RR) and the second recruitment domain is AZip (EE). In some embodiments, different leucine zipper domains are used, for example, SYNZIP 1 to SYNZIP 48, and BATF, FOS, ATF4, ATF3, BACH1, JUND, NFE2L3, and HEPTAD.

In some embodiments, a recruitment domain comprises FK506 binding protein (FKBP); calcineurin catalytic subunit A (CnA); cyclophilin; FKBP-rapamycin associated protein (FRB); gyrase B (GyrB); dihydrofolate reductase (DHFR); DmrB; PYL; ABI; Cry2; CIP; GAI; GID1; or a fragment thereof.

Polynucleotides Encoding Inducible Cell Receptors

In another aspect, the present disclosure provides a polynucleotide encoding an inducible cell receptor provided herein, and a vector comprising such a polynucleotide. When the inducible cell receptor is a multichain receptor, a set of polynucleotides is used. In this case, the set of polynucleotides can be cloned into a single vector or a plurality of vectors. In some embodiments, the polynucleotide comprises a sequence encoding a CAR, wherein the sequence encoding an extracellular protein binding domain is contiguous with and in the same reading frame as a sequence encoding an intracellular signaling domain and a transmembrane domain.

The polynucleotide can be codon optimized for expression in a mammalian cell. In some embodiments, the entire sequence of the polynucleotide has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

The polynucleotide encoding an inducible cell receptor can be obtained using recombinant methods known in the art, such as, for example by screening libraries from cells expressing the polynucleotide, by deriving it from a vector known to include the same, or by isolating directly from cells and tissues containing the same, using standard techniques. Alternatively, the polynucleotide can be produced synthetically, rather than cloned.

The polynucleotide can be cloned into a vector. In some embodiments, an expression vector known in the art is used. Accordingly, the present disclosure includes retroviral and lentiviral vector constructs expressing a CAR that can be directly transduced into a cell.

The present disclosure also includes an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”) (e.g., a 3′ and/or 5′ UTR described herein), a 5′ cap (e.g., a 5′ cap described herein) and/or Internal Ribosome Entry Site (IRES) (e.g., an IRES described herein), the nucleic acid to be expressed, and a polyA tail. RNA so produced can efficiently transfect different kinds of cells. In some embodiments, an RNA CAR vector is transduced into a cell, e.g., a T cell or a NK cell, by electroporation.

Cells

In one aspect, the present disclosure provides CAR-modified cells. The cells can be stem cells, progenitor cells, and/or immune cells modified to express a CAR described herein. In some embodiments, a cell line derived from an immune cell is used. Non-limiting examples of cells, as provided herein, include mesenchymal stem cells (MSCs), natural killer (NK) cells, NKT cells, innate lymphoid cells, mast cells, eosinophils, basophils, macrophages, neutrophils, mesenchymal stem cells, dendritic cells, T cells (e.g., CD8+T cells, CD4+T cells, gamma-delta T cells, and T regulatory cells (CD4+, FOXP3+, CD25+)) and B cells. In some embodiments, the cell a stem cell, such as pluripotent stem cell, embryonic stem cell, adult stem cell, bone-marrow stem cell, umbilical cord stem cells, or other stem cell.

The cells can be modified to express an inducible cell receptor provided herein. The inducible cell receptor can comprise a single chain receptor (i.e., a single fusion protein) or a multichain receptor (i.e., multiple fusion proteins). When the inducible cell receptor is a multichain receptor, the cells comprise multiple fusion proteins. Accordingly, the present disclosure provides a cell (e.g., a population of cells) engineered to express a chimeric antigen receptor (CAR), wherein the CAR comprises an antigen-binding domain, a transmembrane domain, and an intracellular signaling domain.

Pharmaceutical Composition

Pharmaceutical compositions of the present disclosure can comprise an inducible cell receptor (e.g., a CAR) or a cell expression the inducible cell receptor (e.g., a plurality of CAR-expressing cells), as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Pharmaceutical compositions of the present disclosure can be administered in a manner appropriate to the disease to be treated (or prevented). The quantity and frequency of administration can be determined by such factors as the condition of the patient, and the type and severity of the patient's disease, although appropriate dosages may be determined by clinical trials.

In preferred embodiments, the pharmaceutical composition is substantially free of a contaminant, such as endotoxin, mycoplasma, replication competent lentivirus (RCL), p24, VSV-G nucleic acid, HIV gag, residual anti-CD³/anti-CD28 coated beads, mouse antibodies, pooled human serum, bovine serum albumin, bovine serum, culture media components, vector packaging cell or plasmid components, a bacterium and a fungus. The pharmaceutical composition can be free from bacterium such as Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

Method of Preparing Therapeutic Cells

In one aspect, the present disclosure provides a method of preparing a modified immune cells comprising an inducible cell receptor (e.g., CAR-modified cells) for experimental or therapeutic use.

Ex vivo procedures for making therapeutic CAR-modified cells are well known in the art. For example, cells are isolated from a mammal (e.g., a human) and genetically modified (i.e., transduced or transfected in vitro) with a vector expressing a CAR disclosed herein. The CAR-modified cell can be administered to a mammalian recipient to provide a therapeutic benefit. The mammalian recipient may be a human and the CAR-modified cell can be autologous with respect to the recipient. Alternatively, the cells can be allogeneic, syngeneic or xenogeneic with respect to the recipient. The procedure for ex vivo expansion of hematopoietic stem and progenitor cells is described in U.S. Pat. No. 5,199,942, incorporated herein by reference, can be applied to the cells of the present disclosure. Other suitable methods are known in the art, therefore the present disclosure is not limited to any particular method of ex vivo expansion of the cells. Briefly, ex vivo culture and expansion of immune effector cells (e.g., T cells, NK cells) comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

Method of Use

In one aspect, the present disclosure provides a type of cell therapy where immune cells are genetically modified to express an inducible cell receptor provided herein and the modified immune cells are administered to a subject in need thereof.

In some embodiments, the methods comprise culturing the population of cells (e.g. in cell culture media) to a desired cell density (e.g., a cell density sufficient for a particular cell-based therapy). In some embodiments, the population of cells are cultured in the absence of an agent that represses activity of the repressible protease or in the presence of an agent that represses activity of the repressible protease.

In some embodiments, the method comprises administering an agent that represses activity of the repressible protease after administration of the modified immune cells. In some embodiments, the method further comprises withdrawal of an agent that represses activity of the repressible protease after administration of the modified immune cells.

In some embodiments, administration of the agent to a subject induces degradation of a product encoded by the gene of interest. In some embodiments, administration of the agent protects a product encoded by the gene of interest from degradation. In some embodiments, withdrawal of the agent from a subject induces degradation of a product encoded by the gene of interest. In some embodiments, withdrawal of the agent from a subject products a product encoded by the gene of interest from degradation.

In some embodiments, administration of the agent to a subject induces activation of a product encoded by the gene of interest. In some embodiments, administration of the agent induces inhibition of a product encoded by the gene of interest. In some embodiments, withdrawal of the agent from a subject induces activation of a product encoded by the gene of interest. In some embodiments, withdrawal of the agent from a subject induces inhibition of a product encoded by the gene of interest.

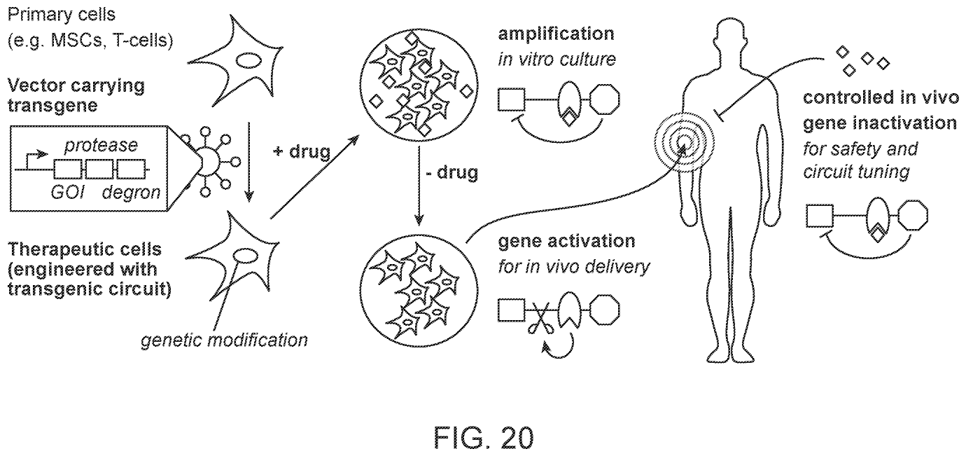

In some embodiments, the population of cells are cultured in the presence of an agent that represses activity of the repressible protease to degrade a product encoded by the gene of interest to produce an expanded population of cells. As shown, for example, in FIG. 20, the sequence encoding the self-excising degron may be positioned at the C-terminal end of the gene of interest (GOI) such that when the cells are cultured in the presence of the agent (that represses activity of the repressible protease), the protease is inactivated and unable to cleave the cognate cleavage site that separates, for example, the C-terminal end of the gene of interest from the degradation sequence. Thus, the degradation sequence remains fused to the gene of interest and promotes degradation of the encoded product (e.g., protein) through either the proteasome or an autophagy-lysosome pathway. This is particularly advantageous, for example, if the gene of interest encodes a product that is toxic to the cells or inhibits cell survival and/or proliferation/expansion of the cells.

In some embodiments, the population of cells is cultured for a period of time that results in the production of an expanded cell population that comprises at least 2-fold the number of cells of the starting population. In some embodiments, the population of cells is cultured for a period of time that results in the production of an expanded cell population that comprises at least 4-fold the number of cells of the starting population. In some embodiments, the population of cells is cultured for a period of time that results in the production of an expanded cell population that comprises at least 16-fold the number of cells of the starting population.

In some embodiments, the methods further comprise removing the agent from the expanded population of cells. The agent may be removed, for example, by simply washing the cells with fresh culture media. In the absence of the agent, the cell are able to produce the protein of interest, e.g., in vivo following administration of the cells to a subject in need.

Thus, in some embodiments, the methods comprise delivering cells of the expanded population of cells to a subject in need of a cell-based therapy. In some embodiments, the subject is a human subject. In some embodiments, the subject in need has an autoimmune condition. In some embodiments, the subject in need has a cancer (e.g., a primary cancer or a metastatic cancer).

Thus, in some embodiments, the gene of interest encodes a therapeutic protein. Examples of therapeutic proteins include, but are not limited to, antibodies, Fc fusion proteins, anticoagulants, blood factors, bone morphogenetic proteins, engineered protein scaffolds, enzymes, growth factors, hormones, interferons, interleukins, and thrombolytics.

The methods, in some embodiments, may comprise administering to the subject an agent that represses activity of the repressible protease to degrade a product encoded by the gene of interest. The agent may be administered any time following administration of the cell-based therapy (the expanded cells containing the gene of interest). In some embodiments, the agent is administered 1 week, 2 weeks, 3 weeks, 1 month, 2 months, 3 months, 6 months, 9 months, 1 year, 2 years, 3 years, 4 years, or 5 years after the subject has received the cell-based therapy. In some embodiments, the agent is administered depending on the health condition of the subject.

Also provided herein are methods of controlling in vivo gene expression in a subject, comprising delivering to a subject in need of a cell-based therapy a population of cells that comprise a nucleic acid that comprises a gene of interest fused to a sequence encoding self-excising degron, wherein the self-excising degron comprises a repressible protease, a cognate cleave site, and a degradation sequence, and administering to the subject an agent that represses activity of the repressible protease to degrade a product encoded by the gene of interest. In some embodiments, the gene of interest is a therapeutic protein.

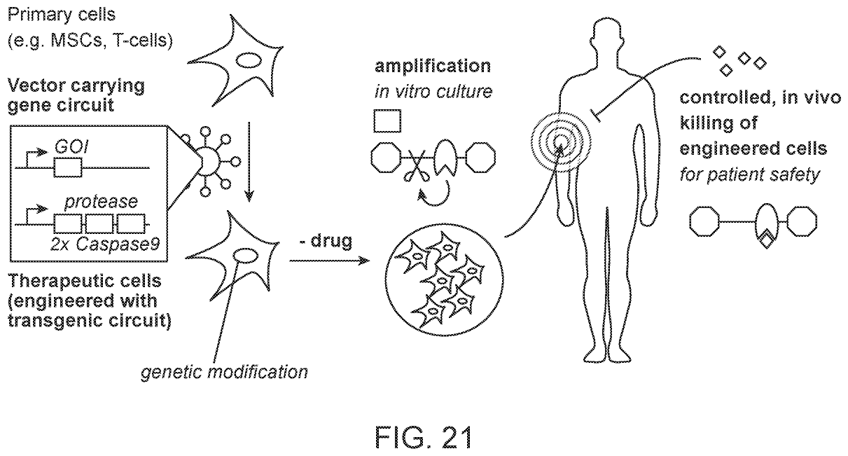

The methods, in other embodiments, comprise providing a population of cells that comprises (a) a nucleic acid that comprises a gene of interest and (b) a nucleic acid that comprises a repressible protease, a cognate cleavage site, and a gene encoding a cell death protein, wherein cleavage of the cognate cleavage site by the repressible protease inhibits activity of the cell death protein. The population of cells are typically first cultured to a desired cell density to produce an expanded population of cells, then the cells, as provided above, are administered to a subject in need of a cell-based therapy. These methods that use a gene encoding a cell death protein are particularly useful for controlling survival of cells of a cell-based therapy following in vivo administration of the cells. As depicted, for example, in FIG. 21, the cells comprise a gene of interest (e.g., encoding a therapeutic protein) that is accompanied by a “kill switch,” which can be activated in vivo by delivering to the subject agent that represses activity of the repressible protease. Thus, following the cell-based therapy, the cells can be forced to undergo apoptosis by activating the kill switch in vivo to produce the cell death protein and kill the cells of the expanded population.

In some embodiments, the cell death protein is a caspase protein. For example, the caspase protein may caspase 9. In some embodiments, more than one copy of a caspase protein, or more than one type of caspase protein, is encoded with the repressible protease and cognate cleavage site. Other cell death proteins and molecules are encompassed by the present disclosure.

In some embodiments, the gene of interest encodes a protein other than a “kill switch.” For example, proteins expressed from the gene of interest can be activated by administration of the protease inhibitor in vivo to induce a desired immune response. In this case, the method may comprise administration to the subject an agent that represses activity of the repressible protease to prevent cleavage of a product encoded by the gene of interest.

In some embodiments, the method can comprise the step of withdrawing an agent that represses activity of the repressible protease from a subject. The agent may be withdrawn any time following administration of the cell-based therapy (the expanded cells containing the gene of interest). In some embodiments, the agent is withdrawn 1 week, 2 weeks, 3 weeks, 1 month, 2 months, 3 months, 6 months, 9 months, 1 year, 2 years, 3 years, 4 years, or 5 years after the subject has received the cell-based therapy. In some embodiments, the agent is withdrawn for 1 week, 2 weeks, 3 weeks, 1 month, 2 months, 3 months, 6 months, 9 months, 1 year, 2 years, 3 years, 4 years, or 5 years. In some embodiments, the agent is withdrawn depending on the health condition of the subject.

The CAR-modified cells of the present disclosure may be administered either alone, or as a pharmaceutical composition in combination with diluents and/or with other components such as IL-2 or other cytokines or cell populations.

### EXAMPLES

The following examples are provided by way of illustration not limitation.

### Example 1

### Impact of Self-Excising Degron on Regulation of YFP Protein Expression in Immortalized Human T Lymphocyte (Jurkat) Cells

A target protein (e.g., YFP) fused to a self-excising degron disclosed herein was tested in vitro in Jurkat cells in the presence and absence of the protease inhibitor Asunaprevir (ASV). Jurkat cells were stably transduced with lentivirus encoding YFP fused to a self-excising degron. The self-excising degron (SEQ ID NO: 4) used in this example encoded the following components arranged from N-terminus to C-terminus: a Hepatitis C (HCV) NS4A-NS4B protease cleavage site, a Flag tag, a HCV NS3 protease domain, a partial HCV NS3 helicase domain sequence and a sequence derived from the HCV NS4A protein. Lentivirus-infected cells were exposed to no ASV, 1 μM ASV or 2 μM ASV for 2, 3 days, 6 days or 7 days. Mean YFP fluorescence of each cell population was measured using flow cytometry.

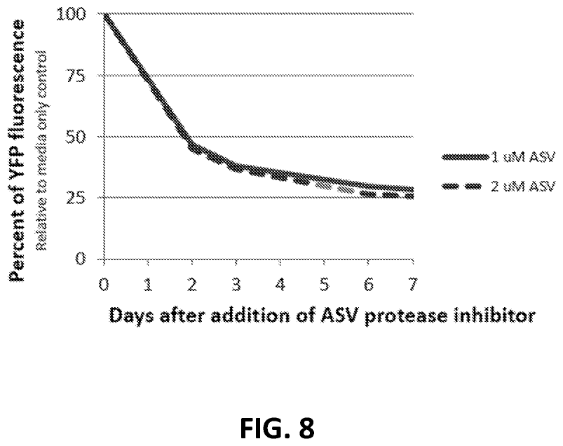

As shown in FIG. 8, a decrease in the intensity of YFP fluorescence was detected after 1 μM and 2 μM ASV treatment at all time points tested (i.e.: Day 2, Day 3, Day 6 and Day 7) compared to no ASV treatment. Notably, YFP fluorescence was reduced nearly fourfold with 7 days of 1 μM or 2 μM ASV treatment. These results indicate that inhibition of protease activity in the self-excising degron could be used to decrease expression of a fused target protein (e.g., YFP) and that a self-excising degron could be used to regulate target protein expression in immortalized human T lymphocyte cells.

### Example 2

### Impact of Self-Excising Degron on Regulation of Anti-HER2 CAR Expression in Jurkat Cells

An anti-HER2 CAR fused to a self-excising degron was tested in vitro in Jurkat cells in the presence and absence of the protease inhibitor Asunaprevir (ASV). The anti-HER2 CAR used in this example has a H3B1 anti-Her2 scFv. A MYC-tagged anti-HER2 CAR was fused to a self-excising degron (SMASh tag, SEQ ID NO: 4 as described in Example 1) and cloned into a lentiviral expression vector. The lentiviral expression was then used to transduce Jurkat cells. Lentivirus-infected cells were treated with no ASV protease inhibitor, 1 μM ASV or 2 μM ASV for three days to determine the effect of protease inhibition on expression of the anti-HER2 CAR. Cells were stained with an anti-MYC fluorescent antibody and the mean fluorescence of each cell population was measured using flow cytometry.

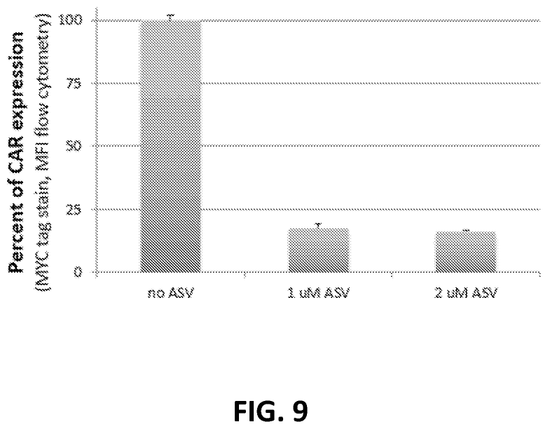

As shown in FIG. 9, a fourfold reduction in expression of the anti-HER2 CAR was detected after 1 μM ASV and after 2 μM ASV treatment compared to no ASV treatment. Thus, these results indicated that inhibition of protease activity in the self-excising degron could promote degradation of the fused CAR protein and further suggest that a self-excising degron could be used to modulate expression of a fused CAR protein (e.g., anti-HER2 CAR) in immortalized human T lymphocyte cells.

### Example 3

### Switchable anti-Her 2 CARs Function to Regulate T Cell Activation

The function of an anti-Her2 CAR fused to a self-excising degron in T cells was tested in vitro. Jurkat cells (an immortalized human T cell line) were transduced with an anti-Her2 CAR fused to a self-excising degron (SEQ ID NO: 4). These cells were incubated in the presence (1 μM ASV) or absence of the ASV protease inhibitor for 2 days, then subsequently placed on tissue culture plates coated with recombinant Her2 (low=2.5 μg/mL; high=10 μg/mL). Following overnight incubation, the ant-Her2 CAR T cells were stained with an anti-CD69 fluorescent antibody, and the mean fluorescence of each cell population was measured using flow cytometry. CD69 was used as a T-cell activation marker.

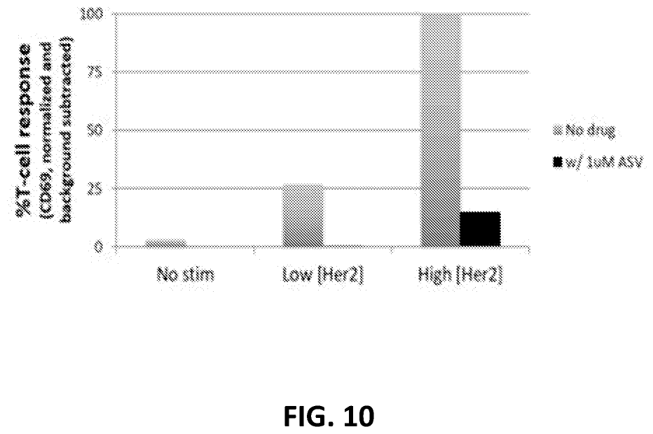

As shown in FIG.10, in the absence of ASV, all of the anti-Her2 CAR T cells were activated, while less than 25% of the cells were activated in the presence of ASV, even when the cells were incubated in the presence of a high concentration of recombinant Her2 protein. Thus, these results demonstrate functional regulation of the CAR switches of the present disclosure and concomitant regulation of T cell activation.

### Example 4

### Characterization of Switchable CARs

### CAR Designs

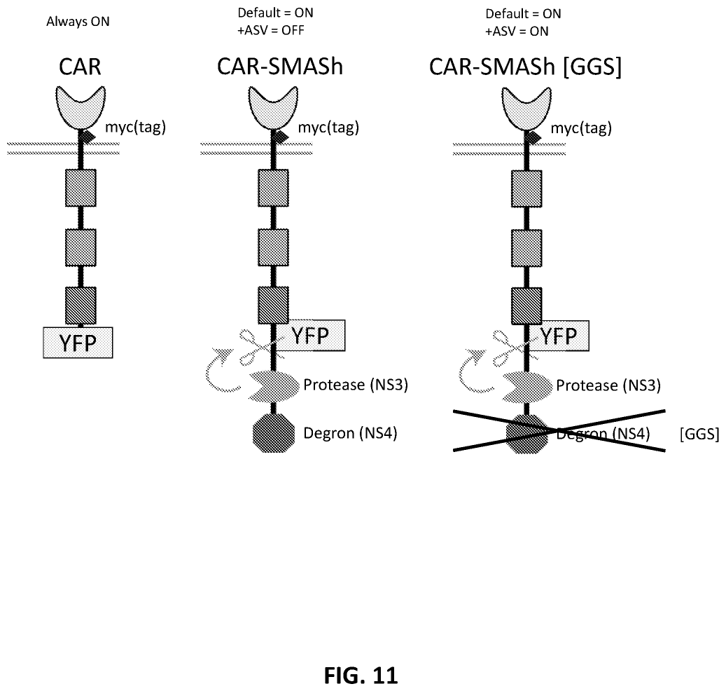

Three fusion proteins were generated for functional studies of a switchable CAR as provided in FIG. 11. The first fusion protein (left) comprises an extracellular protein binding domain fused to a myc tag and three intracellular signaling domains fused to YFP. The second fusion protein (middle; “CAR-SMASh”) further comprises a Hepatitis C (HCV) NS4A-NS4B protease cleavage site, a HCV NS3 protease domain, and a degron derived from the HCV NS4A protein. The CAR-SMASh, therefore, expresses CAR, but induces degradation of CAR in the presence of ASV. The third fusion protein (right; “CAR-SMASh[GGS]”) is a CAR-SMASh mutant lacking the NS4 degron. CAR-SMASh[GGS] also express CAR, but unlike CAR-SMASh, CAR-SMASh[GGS] does not induce degradation of the expressed CAR with or without ASV.

### In Vitro Expression of CAR

Human naïve pan T cells were isolated from PBMC donors by magnetic-assisted cell sorting using StemCell Technologies Total Pan T Cell Isolation Kit and stimulated with anti-CD3/28 Dynabeads at a 1:3 ratio (T cells:Dynabeads), 10\hat{ }6 T cells with 3×10\hat{ }6 Dynabeads, in CTS OpTmizer media with CTS Serum Replacement and recombinant human IL-2 [100 U/ml]. One day later T cells were transduced with lentivirus carrying anti-HER2 scFv CAR or anti-CD19 scFv CAR of three different forms provided in FIG. 10. Specifically, lentivirus encoding CAR, CAR-SMASh and CAR-SMASh[GGS] comprising anti-HER2 or anti-CD19 was transduced. CAR-T cells were then split into a larger well with fresh media added.

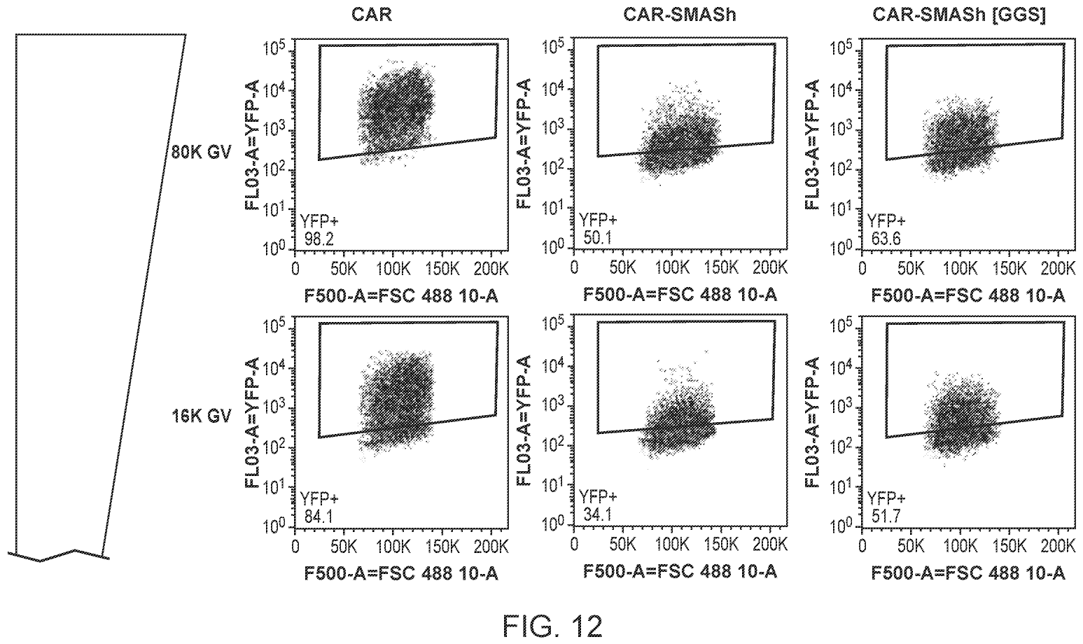

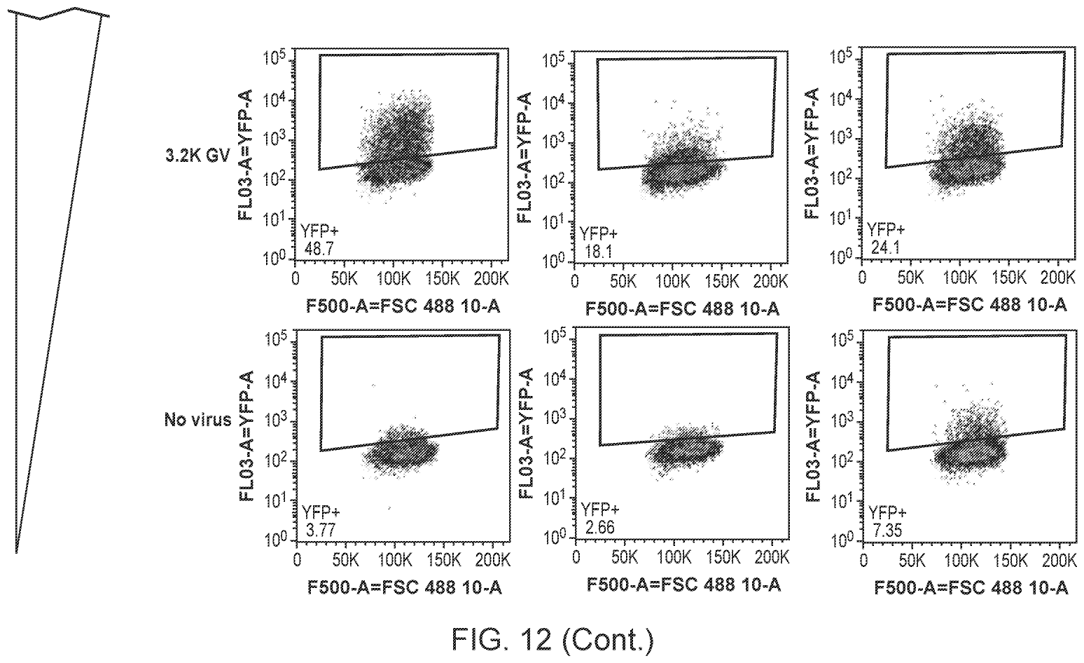

On Day 5 CAR-T cells were acquired by flow cytometry and YFP expression was measured. FIG. 12 shows the FACS analysis results with lentivirus titer (GV) equalized between the CAR designs. The results show that CAR expression correlates with the lentivirus titer (GV) as measured by YFP+ percentage. Similar results were obtained in both CD3+ CD4+and CD3+CD8+ CAR-T cells.

Switchable expression of CAR by application of ASV

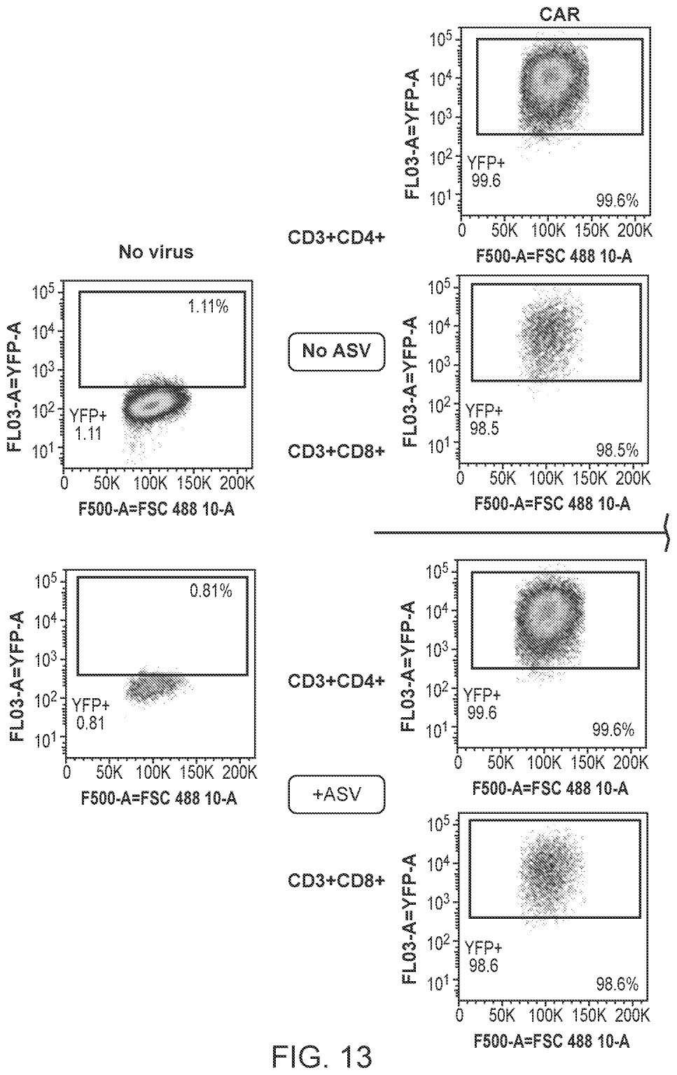

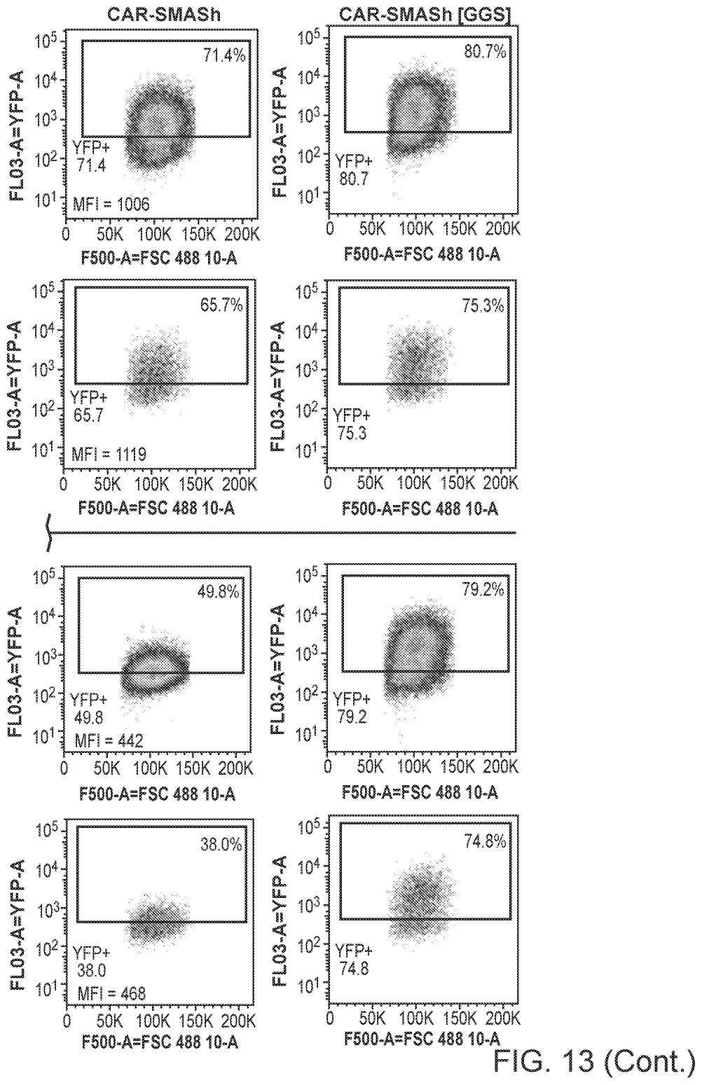

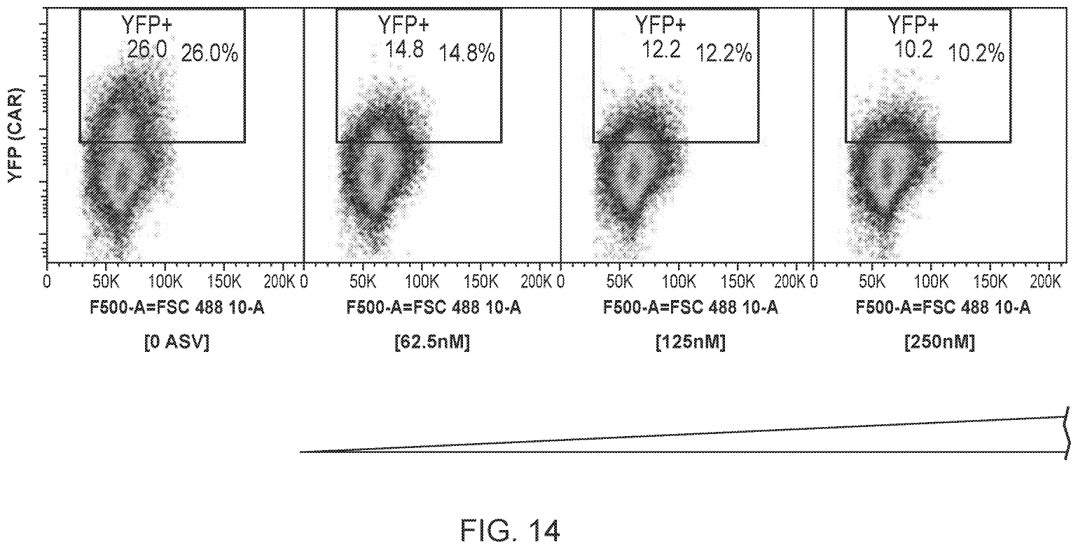

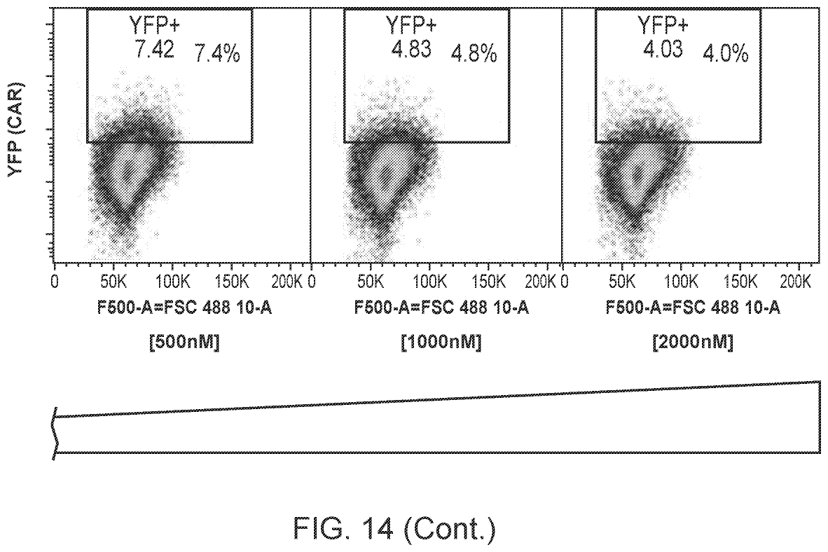

Human total pan T cells were isolated from PBMC donors by magnetic-assisted cell sorting using StemCell Technologies Total Pan T Cell Isolation Kit and stimulated with anti-CD3/28 Dynabeads at a 1:3 ratio (T cells:Dynabeads), 10\hat{ }6 T cells with 3×10\hat{ }Dynabeads, in CTS OpTmizer media with CTS Serum Replacement and recombinant human IL-2 [100 U/ml]. One day later T cells were then transduced with lentivirus carrying a CAR comprising anti-HER2 or anti-CD19 scFv. Specifically, lentivirus encoding CAR, CAR-SMASh and CAR-SMASh[GGS] comprising anti-HER2 or anti-CD19 scFv was transduced. CAR-T cells were then split into a larger well with fresh media added. On Day 5 CAR-T cells were exposed to ASV [2 μM] and then analyzed by flow cytometry to measure YFP expression at various time points, including on day 6 (FIG. 13) and on day 7 (FIG. 14).

As provided in FIG. 13, addition of asunaprevir (ASV) induced SMASh-shutdown of CAR expression as measured by percentage YFP+ and YFP mean fluorescence intensity (MFI) of fluorescence-tagged CAR proteins. CAR-SMASh T cells reduced CAR expression in both CD3+CD4+ and CD3+CD8+ populations while no effect was seen on CAR T cells or a mutant CAR-SMASh [GGS] that lacked the NS4 degron. Furthermore, results provided in FIG. 14 show that reduction of CAR expression becomes more significant when exposed to increasing concentrations of ASV, indicating that SMASh-shutdown of CAR-SMASh expression correlates with concentration of asunaprevir (ASV).

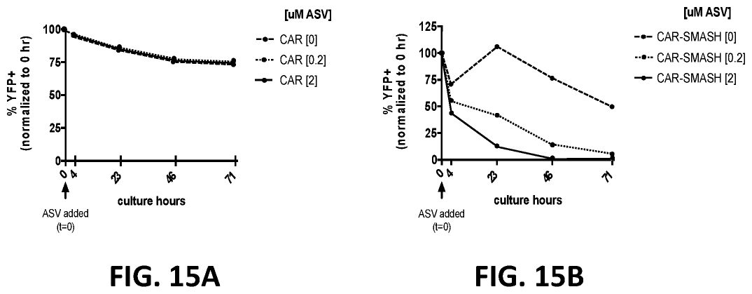

CAR expression levels measured by percentage YFP+of fluorescence-tagged CAR proteins at the indicated time points after addition of asunaprevir (ASV) are further summarized in FIGS. 15A-B. Addition of asunaprevir (ASV) at 0.2 μM or 2 μM concentrations induced SMASh-shutdown of CAR in T cells transduced with CAR-SMASh in a dose dependent manner over time (FIG. 15B). However, addition of asunaprevir (ASV) had no effect in T cells expressing CAR (FIG. 15A).

### Switchable Expression of CAR by Removal of ASV

Human total pan T cells were isolated from PBMC donors by magnetic-assisted cell sorting using StemCell Technologies Total Pan T Cell Isolation Kit and stimulated with anti-CD3/28 Dynabeads at a 1:3 ratio (T cells:Dynabeads), 10\hat{ }6 T cells with 3×10\hat{ }6 Dynabeads, in CTS OpTmizer media with CTS Serum Replacement and recombinant human IL-2 [100 U/ml]. One day later T cells were then transduced with lentivirus carrying a CAR comprising anti-HER2 or anti-CD19 scFv. Specifically, lentivirus encoding CAR, CAR-SMASh and CAR-SMASh[GGS] comprising anti-HER2 or anti-CD19 scFv was transduced. CAR-T cells were then split into a larger well with fresh media added. On Day 5 CAR-T cells were exposed to ASV for 2 days, washed 2 times, then re-cultured in media without ASV and then were acquired by flow cytometry at various time points and YFP expression was measured.

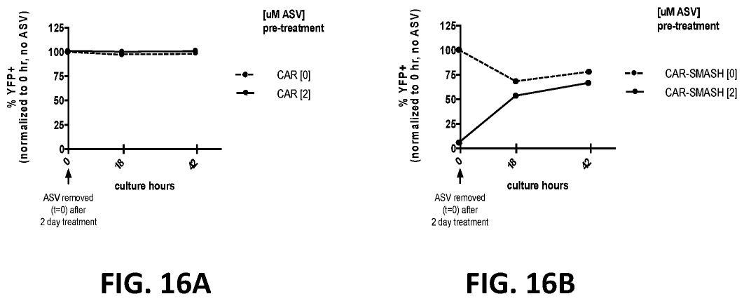

CAR expression levels measured by percentage YFP+ of fluorescence-tagged CAR proteins at the indicated time points after removal of asunaprevir (ASV) are provided in FIGS. 16A-B. The results show that pretreatment with asunaprevir (ASV) induces SMASh-shutdown of CAR expression then removal of ASV allows CAR expression recovery in T cells transduced with CAR-SMASh (FIG. 16B). These reversible effects of asunaprevir (ASV) were not observed in T cells transduced with CAR (FIG. 16A).

### Cytotoxic Effects of Switchable CAR-Expressing T Cells

Human naïve pan T cells were isolated from PBMC donors by magnetic-assisted cell sorting using StemCell Technologies Total Pan T Cell Isolation Kit and stimulated with anti-CD3/28 Dynabeads at a 1:3 ratio (T cells:Dynabeads), 10\hat{ }6 T cells with 3×10\hat{ }6 Dynabeads, in CTS OpTmizer media with CTS Serum Replacement and recombinant human IL-2 [100 U/ml]. One day later T cells were then transduced with lentivirus carrying a CAR-SMASh comprising anti-HER2 scFv. CAR-T cells were then split into a larger well with fresh media added. On Day 8, CAR-T cells were co-cultured with target HER2+ SKOV3 tumor cells at the indicated E:T ratios, incubated overnight, and supernatants collected the next day and cytotoxic killing was measured by LDH assay absorbance in a plate reader.

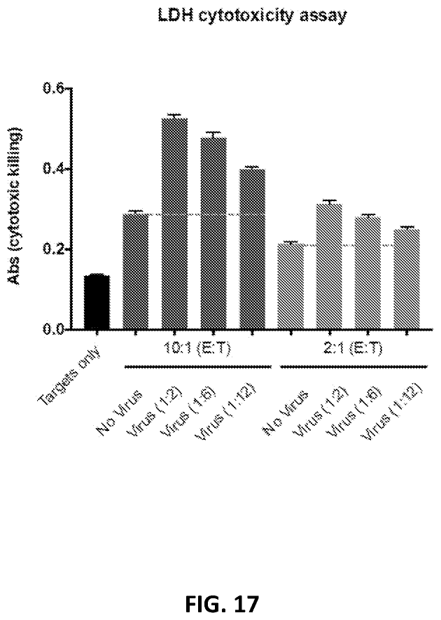

The results are provided in FIG. 17, which show that CAR-SMASh T cells demonstrated killing of target tumor cells as measured by LDH assay. Cytotoxic activity by CAR-SMASh T cells was titratable based on effector-to-target ratio (E:T) of T cells to target tumor cells as well as by virus titer amount used to transduce CAR-SMASh expression into T cells.

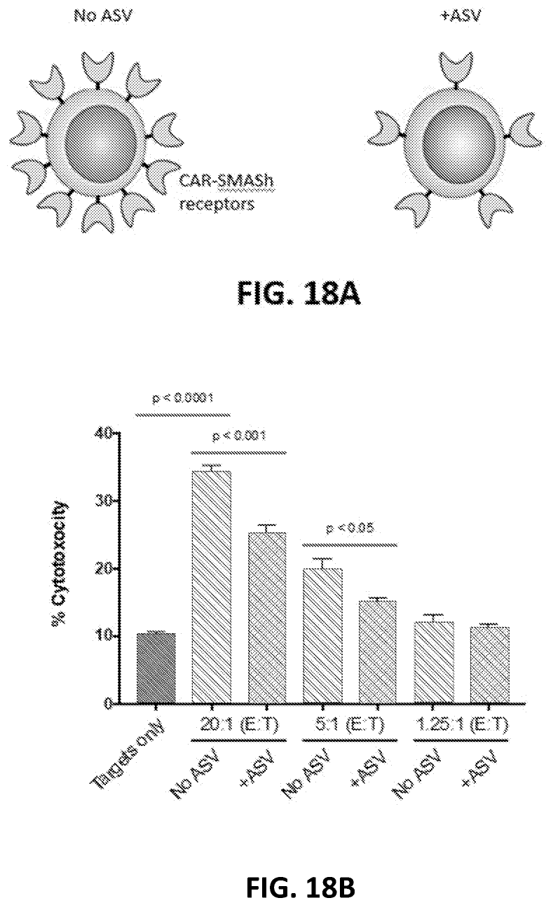

The results were further compared between conditions with and without asunaprevir (ASV) at various effector-to-target (E:T) ratios as provided as FIG. 18B. Treatment of CAR-SMASh T cells but not CAR T cells with asunaprevir (ASV) resulted in lowered cytotoxic killing of target tumor cells as measured by LDH assay at various effector-to-target (E:T) ratios. These results correlated with reduced expression of CAR-SMASh on T cells in the presence of asunaprevir (ASV) as illustrated in FIG. 18A.

### Cytotoxic Production by Switchable CAR-Expressing T Cells

Human naïve pan T cells were isolated from PBMC donors by magnetic-assisted cell sorting using StemCell Technologies Total Pan T Cell Isolation Kit and stimulated with anti-CD3/28 Dynabeads at a 1:3 ratio (T cells:Dynabeads), 10\hat{ }6 T cells with 3×10\hat{ }6 Dynabeads, in CTS OpTmizer media with CTS Serum Replacement and recombinant human IL-2 [100 U/ml]. 1 day later T cells were then transduced with lentivirus carrying anti-HER2 scFv CAR-SMASh designs. CAR-T cells were then split into a larger well with fresh media added. On Day 6 CAR-T cells were treated with ASV [2 μM] for 2 days. On Day 8 CAR-T cells were washed, replenished with ASV, and co-cultured with target HER2+ SKOV3 tumor cells at (10:1) effector-to-target (E:T) ratio, incubated overnight, and supernatants collected the next day. Cytokines were measured by Luminex multi-cytokine array using a Luminex MagPix (Sigma Millipore).

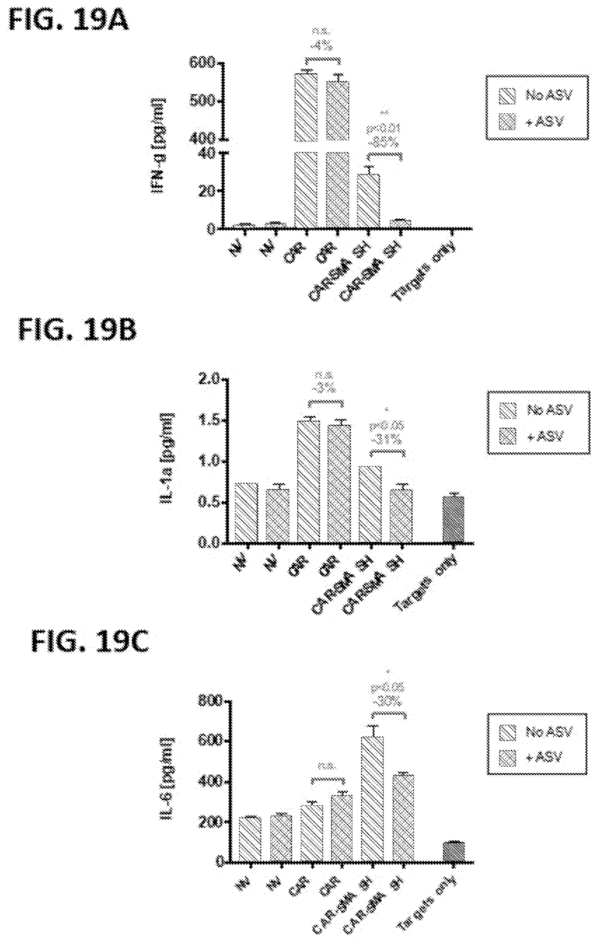

CAR-SMASh T cells, but not CAR T cells, treated with asunaprevir (ASV) had decreased cytotoxicity in the co-culture with target tumor cells, as demonstrated by the decreased production of various cytokines, such as IFN-gamma (FIG. 19A), IL-lalpha (FIG. 19B), and IL-6 (FIG. 19C).

### Incorporation by Reference

All publications, patents, patent applications and other documents cited in this application are hereby incorporated by reference in their entireties for all purposes to the same extent as if each individual publication, patent, patent application or other document were individually indicated to be incorporated by reference for all purposes.

### Equivalents

While various specific embodiments have been illustrated and described, the above specification is not restrictive. It will be appreciated that various changes can be made without departing from the spirit and scope of the present disclosure. Many variations will become apparent to those skilled in the art upon review of this specification.

SEQUENCE LISTING

| SEQ ID NO (description) | SEQUENCE |
|---|---|
| SEQ ID NO: 1 | PITKIDTKYIMTCMSADLEVVTSTWVLVGGVLAALAAYCL |
| (Degradation sequences) | ST |
| SEQ ID NO: 2 | DEMEECSQHL |
| (HCV NS4A/4B protease | |
| cleavage site) | |
| SEQ ID NO: 3 | EDVVPCSMG |
| (HCV NS5A/5B protease | |
| cleavage site) | |
| SEQ ID NO: 4 | DEMEECSQHLPGAGSSGDIMDYKDDDDKGSSGTGSGSGTS |
| (C-terminal degradation | APITAYAQQTRGLLGCIITSLTGRDKNQVEGEVQIVSTATQT |
| signal with NS4A/4B | FLATCINGVCWAVYHGAGTRTIASPKGPVIQMYTNVDQDL |
| protease cleavage site) | VGWPAPQGSRSLTPCTCGSSDLYLVTRHADVIPVRRRGDSR |
| | GSLLSPRPISYLKGSSGGPLLCPAGHAVGLFRAAVCTRGVA |
| | KAVDFIPVENLETTMRSPVFTDNSSPPAVTLTHPITKIDTKYI |
| | MTCMSADLEVVTSTWVLVGGVLAALAAYCLSTGCVVIVG |
| | RIVLSGKPAIIPDREVLY |
| SEQ ID NO: 5 | MDYKDDDDKGSSGTGSGSGTSAPITAYAQQTRGLLGCIITS |
| (N-terminal degradation | LTGRDKNQVEGEVQIVSTATQTFLATCINGVCWAVYHGAG |
| signal with HCV NS5A/5B | TRTIASPKGPVIQMYTNVDQDLVGWPAPQGSRSLTPCTCGS |
| protease cleavage site) | SDLYLVTRHADVIPVRRRGDSRGSLLSPRPISYLKGSSGGPL |
| | LCPAGHAVGLFRAAVCTRGVAKAVDFIPVENLETTMRSPV |
| | FTDNS SPPAVTLTHPITKIDTKYIMTCMSADLEVVTSTWVL |
| | VGGVLAALAAYCLSTGCVVIVGRIVLSGKPAGSSGSSTIPDR |
| | EVLYQEFEDVVPCSMG |
| SEQ ID NO: 6 | APITAYAQQTRGLLGCIITSLTGRDKNQVEGEVQIVSTATQT |
| (Hepatitis C Virus) | FLATCINGVCWAVYHGAGTRTIASPKGPVIQMYTNVDQDL |
| | VGWPAPQGSRSLTPCTCGSSDLYLVTRHADVIPVRRRGDSR |
| | GSLLSPRPISYLKGSSGGPLLCPAGHAVGLFRAAVCTRGVA |
| | KAVDFIPVENLETTMRSPVFTD |
| SEQ ID NO: 7 | PQVTLWQRPLVTIKIGGQLKEALLDTGADDTVLEEM SLPGR |
| (HIV-1 protease) | WKPKMIGGIGGFIKVRQYDQILIEICGHKAIGTVLVGPTPVN |
| | IIGRNLLTQIGCTLNF |
| SEQ ID NO: 8 | SGVLWDTPSPPEVERAVLDDGIYRIMQRGLLGRSQVGVGV |
| (DENV NS3pro | FQDGVFHTMWHVTRGAVLMYQGKRLEPSWASVKKDLISY |
| (NS2B/NS3)) | GGGWRFQGSWNTGEEVQVIAVEPGKNPKNVQTAPGTFKT |
| >sp\|P33478\|1475-2093 | PEGEVGAIALDFKPGTSGSPIVNREGKIVGLYGNGVVTTSG |
| | TYVSAIAQAKASQEGPLPEIEDEVFRKRNLTIMDLHPGSGK |
| | TRRYLPAIVREAIRRNVRTLILAPTRVVASEMAEALKGMPIR |
| | YQTTAVKSEHTGKEIVDLMCHATFTMRLLSPVRVPNYNMII |
| | MDEAHFTDPASIARRGYISTRVGMGEAAAIFMTATPPGSVE |
| | AFPQSNAVIQDEERDIPERSWNSGYEWITDFPGKTVWFVPSI |
| | KS GNDIANCLRKNGKRVIQLSRKTFDTEYQKTKNNDWDY |
| | VVTTDISEMGANFRADRVIDPRRCLKPVILKDGPERVILAGP |
| | MPVTVAS AAQRRGRIGRNQNKEGDQYVYMGQPLNNDED |
| | HAHWTEAKMLLDNINTPEGIIPALFEPEREKSAAIDGEYRLR |
| | GEARKTFVELMRRGDLPVWLSYKVASEGFQYSDRRWCFD |
| | GERNNQVLEENMDVEMWTKEGERKKLRPRWLDARTYSD |
| | PLALREFKEFAAGRR |
| SEQ ID NO: 9 | AGVLWDVPSPPPVGKAELEDGAYRIKQKGILGYSQIGAGV |
| (DENV NS3pro | YKEGTFHTMWHVTRGAVLMHKGKRIEPSWADVKKDLISY |
| (NS2B/NS3)) | GGGWKLEGEWKEGEEVQVLALEPGKNPRAVQTKPGLFKT |
| >sp\|P14340\|1476-2093 | NAGTIGAVSLDFSPGTSGSPIIDKKGKVVGLYGNGVVTRSG |
| | AYVSAIAQTEKSIEDNPEIEDDIFRKRKLTIMDLHPGAGKTK |
| | RYLPAIVREAIKRGLRTLILAPTRVVAAEMEEALRGLPIRYQ |
| | TPAIRAEHTGREIVDLMCHATFTMRLLSPVRVPNYNLIIMD |
| | EAHFTDPASIAARGYISTRVEMGEAAGIFMTATPPGSRDPFP |
| | QSNAPIMDEEREIPERSWSSGHEWVTDFKGKTVWFVPSIKA |
| | GNDIAACLRKNGKKVIQLSRKTFDSEYVKTRTNDWDFVVT |
| | TDISEMGANFKAERVIDPRRCMKPVILTDGEERVILAGPMP |
| | VTHSSAAQRRGRIGRNPKNENDQYIYMGEPLENDEDCAHW |
| | KEAKMLLDNINTPEGIIPSMFEPEREKVDAIDGEYRLRGEAR |
| | KTFVDLMRRGDLPVWLAYRVAAEGINYADRRWCFDGIKN |
| | NQILEENVEVEIWTKEGERKKLKPRWLDAKIYSDPLALKEF |
| | KEFAAGRK |
| SEQ ID NO: 10 | SGVLWDVPSPPETQKAELEEGVYRIKQQGIFGKTQVGVGV |
| (DENV NS3pro | QKEGVFHTMWHVTRGAVLTHNGKRLEPNWASVKKDLISY |
| (NS2B/NS3)) | GGGWRLSAQWQKGEEVQVIAVEPGKNPKNFQTMPGIFQTT |
| >sp\|Q99D35\|1474-2092 | TGEIGAIALDFKPGTSGSPIINREGKVVGLYGNGVVTKNGG |
| | YVSGIAQTNAEPDGPTPELEEEMFKKRNLTIMDLHPGSGKT |
| | RKYLPAIVREAIKRRLRTLILAPTRVVAAEMEEALKGLPIRY |
| | QTTATKSEHTGREIVDLMCHATFTMRLLSPVRVPNYNLIIM |
| | DEAHFTDPASIAARGYISTRVGMGEAAAIFMTATPPGTADA |
| | FPQSNAPIQDEERDIPERSWNSGNEWITDFVGKTVWFVPSIK |
| | AGNDIANCLRKNGKKVIQLSRKTFDTEYQKTKLNDWDFVV |
| | TTDISEMGANFKADRVIDPRRCLKPVILTDGPERVILAGPMP |
| | VTVASAAQRRGRVGRNPQKENDQYIFMGQPLNKDEDHAH |
| | WTEAKMLLDNINTPEGIIPALFEPEREKSAAIDGEYRLKGES |
| | RKTFVELMRRGDLPVWLAHKVASEGIKYTDRKWCFDGER |
| | NNQILEENMDVEIWTKEGEKKKLRPRWLDARTYSDPLALK |
| | EFKDFAAGRK |
| SEQ ID NO: 11 | SGALWDVPSPAATQKAALSEGVYRIMQRGLFGKTQVGVGI |
| (DENV NS3pro | HIEGVFHTMWHVTRGSVICHETGRLEPSWADVRNDMISYG |
| (NS2B/NS3)) | GGWRLGDKWDKEEDVQVLAIEPGKNPKHVQTKPGLFKTL |
| >sp\|Q5UCB8\|1475-2092 | TGEIGAVTLDFKPGTSGSPIINRKGKVIGLYGNGVVTKSGD |
| | YVSAITQAERIGEPDYEVDEDIFRKKRLTIMDLHPGAGKTK |
| | RILPSIVREALKRRLRTLILAPTRVVAAEMEEALRGLPIRYQ |
| | TPAVKSEHTGREIVDLMCHATFTTRLLSSTRVPNYNLIVMD |
| | EAHFTDPSSVAARGYISTRVEMGEAAAIFMTATPPGTTDPF |
| | PQSNSPIEDIEREIPERSWNTGFDWITDYQGKTVWFVPSIKA |
| | GNDIANCLRKSGKKVIQLSRKTFDTEYPKTKLTDWDFVVT |
| | TDISEMGANFRAGRVIDPRRCLKPVILPDGPERVILAGPIPVT |
| | PASAAQRRGRIGRNPAQEDDQYVFSGDPLKNDEDHAHWT |
| | EAKMLLDNIYTPEGIIPTLFGPEREKTQAIDGEFRLRGEQRK |
| | TFVELMRRGDLPVWLSYKVASAGISYKDREWCFTGERNN |
| | QILEENMEVEIWTREGEKKKLRPKWLDARVYADPMALKD |
| | FKEFASGRK |
| SEQ ID NO: 12 | LQMLPESEDEESYDTESEFTEFTEDELPYDDGSLQMLPESED |
| (PEST, Two copies of | EESYDTESEFTEFTEDELPYDD |
| residues 277-307 of IκBa | |
| (human)) | |
| SEQ ID NO: 13 | EIKDKEEVQRKRQKLMPNFSDSFGGGSGAGAGGGGMFGS |
| (GRR, Residues 352-408 of | GGGGGGTGSTGPGYSFPH |
| p105 (human)) | |
| SEQ ID NO: 14 | IDDENGSVILQDDDYDDGNNHIPFEDDDVYNYNDNDDDDE |
| (DRR, Residue 210-295 of | RIEFEDDDDDDDDSIDNDSVMDRKQPHKAEDESEDVEDVE |
| Cdc34 (yeast)) | RVSKKD |
| SEQ ID NO: 15 | PESMREEYRKEGSKRIKCPDCEPFCNKRGSPESMREEYRKE |
| (SNS, Tandem repeat of | |
| SP2 and NB (SP2-NB- | |
| SP2)) | |
| SEQ ID NO: 16 | RSYSPTSPNYSPTSPSGSYSPTSPNYSPTSPSGGSRSYSPTSPN |
| (RPB, (Four copies of | YSPTSPSGSYSPTSPNYSPTSPSG |
| residues 1688-1702 of | |
| RPB1 (yeast)) | |
| SEQ ID NO: 17 | PESMREEYRKEGSSLLTEVETPGSPESMREEYRKEGSSLLTE |
| (SPmix, Tandem repeat of | VETPGSPESMREEYRKE |
| SP1 and SP2 (5P2-SP1- | |
| SP2-SP1-SP2) (Influenza A | |
| virus M2 protein)) | |
| SEQ ID NO: 18 | LIEEVRHRLKTTENSGSLIEEVRHRLKTTENSGSLIEEVRHRL |
| (NS2; Three copies of | KTTENSGS |
| residue 79-93 of Influenza | |
| A virus NS protein) | |
| SEQ ID NO: 19 | FPPEVEEQDDGTLPMSCAQESGMDRHPAACASARINV |
| (ODC; Residue 106-142 of | |
| ornithine decarboxylase) | |
| SEQ ID NO: 20 | SHGFPPEVEEQAAGTLPMSCAQESGMDRHPAACASARINV |
| (mODC DA, amino acids | |
| 422-461 of mODC | |
| (D433A, D434A)) ) -- this | |
| is the most potent mutant | |
| of mODC PEST found in a | |
| screen (29% after 2hr -> | |
| 6%) | |
| SEQ ID NO: 21 | MGAASGRRGPGLLLPLPLLLLLPPQPALALDPGLQPGNFS |
| (angiotensin converting | ADEAGAQLFAQSYNSSAEQVLFQSVAASWAHDTNITAENA |
| enzyme (ACE)) | RRQEEAALLSQEFAEAWGQKAKELYEPIWQNFTDPQLRRI |
| | IGAVRTLGSANLPLAKRQQYNALLSNMSRIYSTAKVCLPN |
| | KTATCWSLDPDLTNILASSRSYAMLLFAWEGWHNAAGIPL |
| | KPLYEDFTALSNEAYKQDGFTDTGAYWRSWYNSPTFEDDL |
| | EHLYQQLEPLYLNLHAFVRRALHRRYGDRYINLRGPIPAH |
| | LLGDMWAQSWENIYDMVVPFPDKPNLDVTSTMLQQGWN |
| | ATHMFRVAEEFFTSLELSPMPPEFWEGSMLEKPADGREVV |
| | CHASAWDFYNRKDFRIKQCTRVTMDQLSTVHHEMGHIQY |
| | YLQYKDLPVSLRRGANPGFHEAIGDVLALSVSTPEHLHKIG |
| | LLDRVTNDTESDINYLLKMALEKIAFLPFGYLVDQWRWGV |
| | FSGRTPPSRYNFDWWYLRTKYQGICPPVTRNETHFDAGAK |
| | FHVPNVTPYIRYFVSFVLQFQFHEALCKEAGYEGPLHQCDI |
| | YRSTKAGAKLRKVLQAGSSRPWQEVLKDMVGLDALDAQP |
| | LLKYFQPVTQWLQEQNQQNGEVLGWPEYQWHPPLPDNYP |
| | EGIDLVTDEAEASKFVEEYDRTSQVVWNEYAEANWNYNT |
| | NITTETSKILLQKNMQIANHTLKYGTQARKFDVNQLQNTTI |
| | KRIIKKVQDLERAALPAQELEEYNKILLDMETTYSVATVCH |
| | PNGSCLQLEPDLTNVMATSRKYEDLLWAWEGWRDKAGR |
| | AILQFYPKYVELINQAARLNGYVDAGDSWRSMYETPSLEQ |
| | DLERLFQELQPLYLNLHAYVRRALHRHYGAQHINLEGPIPA |
| | HLLGNMWAQTWSNIYDLVVPFPSAPSMDTTEAMLKQGWT |
| | PRRMFKEADDFFTSLGLLPVPPEFWNKSMLEKPTDGREVV |
| | CHASAWDFYNGKDFRIKQCTTVNLEDLVVAHHEMGHIQY |
| | FMQYKDLPVALREGANPGFHEAIGDVLALSVSTPKHLHSL |
| | NLLSSEGGSDEHDINFLMKMALDKIAFIPFSYLVDQWRWR |
| | VFDGSITKENYNQEWWSLRLKYQGLCPPVPRTQGDFDPGA |
| | KFHIPSSVPYIRYFVSFIIQFQFHEALCQAAGHTG |
| | PLHKCDIYQSKEAGQRLATAMKLGFSRPWPEAMQLITGQP |
| | NMSASAMLSYFKPLLDWLRTENELHGEKLGWPQYNWTPN |
| | SARSEGPLPDSGRVSFLGLDLDAQQARVGQWLLLFLGIALL |
| | VATLGLSQRLFSIRHRSLHRHSHGPQFGSEVELRHS |
| SEQ ID NO: 22 | EDANSEPLFAERKDABCYL |
| (calpain substrate) | |
| SEQ ID NO: 23 | YVAD |
| (caspase cleavage | |
| sequence) | |
| SEQ ID NO: 24 | VDVAD |
| (caspase cleavage | |
| sequence) | |
| SEQ ID NO: 25 | DEVD |
| (caspase cleavage | |
| sequence) | |
| SEQ ID NO: 26 | VEHD |
| (caspase cleavage | |
| sequence) | |
| SEQ ID NO: 27 | LGHD |
| (caspase cleavage | |
| sequence) | |
| SEQ ID NO: 28 | LQTDG |
| (caspase cleavage | |
| sequence) | |
| SEQ ID NO: 29 | EVNLDAEF |
| (amyloid precursor protein | |
| secretase beta cleavage | |
| sequence) | |
| SEQ ID NO: 30 | PQGIAGQ |
| (MMP 2 cleavage | |
| sequence) | |
| SEQ ID NO: 31 | ENLYFQS |
| (tobacco Etch virus (TEV) | |
| protease cleavage | |
| sequence) | |
| SEQ ID NO: 32 | HPFHLK(DNP) |
| (mast cell chymase, rat | |
| mast cell protease, rat | |
| vascular chymase cleavage | |
| sequence) | |

What is claimed is:

1. A fusion protein comprising:
a. a chimeric antigen receptor (CAR) comprising (a) an extracellular protein binding domain, and (b) a first intracellular signaling domain, and (c) a transmembrane domain located between the extracellular protein binding domain and the first intracellular signaling domain; and
b. a self-excising degron operably linked to the CAR and comprising (a) a repressible protease, (b) a cognate cleavage site, and (c) a degradation sequence.

2. The fusion protein of claim 1, wherein the CAR further comprises a second intracellular signaling domain.

3. The fusion protein of claim 2, wherein the CAR further comprises a third intracellular signaling domain.

4. The fusion protein of any one of claims 1-3, wherein the extracellular protein binding domain is an antibody, an antigen-binding fragment thereof, a F(ab) fragment, a F(ab′) fragment, a single chain variable fragment (scFv), or a single-domain antibody (sdAb).

5. The fusion protein of any one of claims 1-3, wherein the extracellular protein binding domain comprises a ligand-binding domain.

6. The fusion protein of claim 5, wherein the ligand-binding domain is a domain from a receptor, optionally wherein the receptor is selected from the group consisting of TCR, BCR, a cytokine receptor, RTK receptors, serine/threonine kinase receptors, hormone receptors, immunoglobulin superfamily receptors, and TNFR-superfamily of receptors.

7. The fusion protein of claim 6, wherein the receptor is a cytokine receptor selected from IL-1, IL-10, and IL-7, TGF-beta receptor, PD-1 or OX40.

8. The fusion protein of any one of claims 1-7, wherein the self-excising degron is located at the C-terminus of the CAR.

9. The fusion protein of any one of claims 1-8, wherein the self-excising degron comprises the cognate cleavage site, the repressible protease, and the degradation sequence physically linked to one another in the sequential order from the N-terminus to the C-terminus.

10. The fusion protein of any one of claims 1-8, wherein the self-excising degron comprises the repressible protease, the cognate cleavage site, and the degradation sequence physically linked to one another in the sequential order from the N-terminus to the C-terminus.

11. The fusion protein of any one of claims 1-10, further comprising a protease inhibitor bound to the repressible protease.

12. The fusion protein of any one of claims 1-11, further comprising a first recruitment domain.

13. A fusion protein comprising a chimeric antigen receptor (CAR) comprising (a) an extracellular protein binding domain, (b) a first intracellular signaling domain, and (c) a transmembrane domain located between the extracellular protein binding domain and the first intracellular signaling domain, (d) a repressible protease, and (e) a cognate cleavage site of the repressible protease.

14. The fusion protein of claim 13, wherein the CAR further comprises a second intracellular signaling domain.

15. The fusion protein of claim 14, wherein the CAR further comprises a third intracellular signaling domain.

16. The fusion protein of any one of claims 13-15, wherein the extracellular protein binding domain is an antibody, an antigen-binding fragment thereof, a F(ab) fragment, a F(ab′) fragment, a single chain variable fragment (scFv), or a single-domain antibody (sdAb).

17. The fusion protein of any one of claims 13-15, wherein the extracellular protein binding domain comprises a ligand-binding domain.

18. The fusion protein of claim 17, wherein the ligand-binding domain is a domain from a receptor, wherein the receptor is selected from the group consisting of TCR, BCR, a cytokine receptor, RTK receptors, serine/threonine kinase receptors, hormone receptors, immunoglobulin superfamily receptors, and TNFR-superfamily of receptors.

19. The fusion protein of claim 18, wherein the receptor is a cytokine receptor selected from IL-1, IL-10, and IL-7, TGF-beta receptor, PD-1 or OX40.

20. The fusion protein of any one of claims 13-19, wherein the cognate cleavage site is located:
a. between the transmembrane domain and the first intracellular signaling domain;
b. between the extracellular protein binding domain and the transmembrane domain;
c. between the first intracellular signaling domain and the second intracellular signaling domain; or
d. between the second intracellular signaling domain and the third intracellular signaling domain.

21. The fusion protein of claim 20, wherein:
a. the cognate cleavage site and the repressible protease are physically linked to one another in the sequential order from the N-terminus to the C-terminus; or
b. the repressible protease and the cognate cleavage site are physically linked to one another in the sequential order from the N-terminus to the C-terminus.

22. The fusion protein of any one of claims 13-21, wherein the repressible protease is located at the C-terminus of the CAR.

23. The fusion protein of any one of claims 13-22, wherein the CAR further comprises a ligand operably linked to the ligand-binding domain and the cognate cleavage site is located between the ligand-binding domain and the ligand.

24. The fusion protein of claim 23, wherein the repressible protease and the cognate cleavage site are physically linked to one another.

25. The fusion protein of any one of claims 13-24, further comprising a protease inhibitor bound to the repressible protease.

26. A fusion protein comprising a chimeric antigen receptor (CAR) comprising from the C-terminus to the N-terminus: (a) a first intracellular signaling domain, (b) a repressible protease, (c) a cognate cleavage site of the repressible protease, (d) one or more additional intracellular signaling domains, (e) a transmembrane domain, and (f) an extracellular protein binding domain.

27. A fusion protein comprising a chimeric antigen receptor (CAR) comprising from the C-terminus to the N-terminus: (a) a repressible protease, (b) a first intracellular signaling domain, (c) a cognate cleavage site of the repressible protease, (d) one or more additional intracellular signaling domains, (e) a transmembrane domain, and (f) an extracellular protein binding domain.

28. The fusion protein of any one of claims 1-27, wherein the CAR further comprises a spacer domain located between the extracellular protein binding domain and the transmembrane domain.

29. A composition comprising:
a. a first fusion protein comprising: (a) an extracellular protein binding domain, and (b) a first recruitment domain; and
b. a second fusion protein comprising a chimeric antigen receptor (CAR), wherein the CAR comprises: (a) a second recruitment domain, (b) a transmembrane domain, and (c) a first intracellular signaling domain, and (d) a self-excising degron operably linked to the CAR, wherein the self-excising degron comprises (i) a repressible protease, (ii) a cognate cleavage site, and (iii) a degradation sequence.

30. The composition of claim 29, wherein:
a. the first fusion protein is a soluble protein;
b. the first fusion protein is a membrane-bound protein comprising a transmembrane domain, and the first recruitment domain is located between the extracellular protein binding domain and the transmembrane domain; or
c. the first fusion protein is a membrane-bound protein comprising a transmembrane domain, and the transmembrane domain is located between the first recruitment domain and the extracellular protein binding domain.

31. The composition of claim 29 or claim 30, wherein:
a. the CAR comprises from the N-terminus to the C-terminus the second recruitment domain, the transmembrane domain, and the first intracellular signaling domain;
b. the CAR comprises from the N-terminus to the C-terminus the transmembrane domain, the second recruitment domain, and the first intracellular signaling domain; or
c. the CAR comprises from the N-terminus to the C-terminus the transmembrane domain, the first intracellular signaling domain, and the second recruitment domain.

32. The composition of any one of claims 29-31, wherein the CAR further comprises a second intracellular signaling domain, optionally wherein the second intracellular signaling domain is located N-terminal to the first intracellular signaling domain or is located C-terminal to the first intracellular signaling domain.

33. The composition of any one of claims 29-32, wherein the CAR further comprises a second extracellular protein binding domain.

34. The composition of any one of claims 29-33, wherein the extracellular protein binding domain or the second extracellular protein binding domain is an antibody, an antigen-binding fragment thereof, a F(ab) fragment, a F(ab′) fragment, a single chain variable fragment (scFv), or a single-domain antibody (sdAb).

35. The composition of any one of claims 29-33, wherein the extracellular protein binding domain or the second extracellular protein binding domain comprises a ligand-binding domain.

36. The composition of claim 35, wherein the ligand-binding domain is a domain from a receptor, wherein the receptor is selected from the group consisting of TCR, BCR, a cytokine receptor, RTK receptors, serine/threonine kinase receptors, hormone receptors, immunoglobulin superfamily receptors, and TNFR-superfamily of receptors.

37. The composition of claim 36, wherein the receptor is a cytokine receptor selected from IL-1, IL-10, and IL-7, TGF-beta receptor, PD-1 or OX40.

38. The composition of any one of claims 29-37, wherein the self-excising degron is located at the C-terminus of the CAR.

39. The composition of any one of claims 29-38, wherein the self-excising degron comprises:
a. the cognate cleavage site, the repressible protease, and the degradation sequence physically linked to one another in the sequential order from the N-terminus to the C-terminus; or
b. the repressible protease, the cognate cleavage site, and the degradation sequence physically linked to one another in the sequential order from the N-terminus to the C-terminus.

40. The composition of any one of claims 29-39, wherein the first protein further comprises a second self-excising degron, wherein the second self-excising degron comprises (i) a second repressible protease, (ii) a second cognate cleavage site, and (iii) a second degradation sequence operably linked to one another.

41. The composition of any one of claims 29-40, wherein the first protein and the second protein are bound through the first recruitment domain and the second recruitment domain.

42. The composition of any one of claims 29-41, further comprising a protease inhibitor bound to the repressible protease.

43. A composition comprising:
a. a first fusion protein comprising (a) an extracellular protein binding domain, (b) a first recruitment domain, (c) a cognate cleavage site, and (d) a degradation sequence, and
b. a second fusion protein comprising: (a) a transmembrane domain, (b) a second recruitment domain, and (c) a repressible protease.

44. The composition of claim 43, wherein the cognate cleavage site and the degradation sequence are physically linked to one another.

45. The composition of claim 43 or claim 44, wherein the cognate cleavage site and the degradation sequence are located at the C-terminus of the first fusion protein.

46. The composition of any one of claims 43-45, wherein the repressible protease is located at the C-terminus of the second fusion protein.

47. The composition of any one of claims 43-46, wherein the first fusion protein further comprises a first intracellular signaling domain.

48. The composition of any one of claims 43-47, wherein the second fusion protein further comprises a second intracellular signaling domain.

49. The composition of any one of claims 43-48, wherein the second fusion protein further comprises a second extracellular protein binding domain.

50. The composition of any one of claims 43-49, wherein the extracellular protein binding domain or the second extracellular protein binding domain is an antibody, an antigen-binding fragment thereof, a F(ab) fragment, a F(ab′) fragment, a single chain variable fragment (scFv), or a single-domain antibody (sdAb).

51. The composition of any one of claims 43-48, wherein the extracellular protein binding domain or the second extracellular protein binding domain comprises a ligand-binding domain.

52. The composition of claim 51, wherein the ligand-binding domain is a domain from a receptor, wherein the receptor is selected from the group consisting of TCR, BCR, a cytokine receptor, RTK receptors, serine/threonine kinase receptors, hormone receptors, immunoglobulin superfamily receptors, and TNFR-superfamily of receptors.

53. The composition of claim 52, wherein the receptor is a cytokine receptor selected from IL-1, IL-10, and IL-7, TGF-beta receptor, PD-1 or OX40.

54. The composition of any one of claims 43-53, wherein the first fusion protein and the second fusion protein are bound through the first recruitment domain and the second recruitment domain.

55. The composition of any one of claims 43-54, further comprising a protease inhibitor bound to the repressible protease.

56. A composition comprising:
a. a first fusion protein comprising: (a) an extracellular protein binding domain and (b) a first recruitment domain operably linked to the extracellular protein binding domain, and (c) a repressible protease, and
b. a second fusion protein comprising: (a) a first intracellular signaling domain, (b) a second recruitment domain, (c) a cognate cleavage site, and (d) a degradation sequence.

57. The composition of claim 56, wherein the cognate cleavage site and the degradation sequence are physically linked to one another.

58. The composition of claim 56 or claim 57, wherein the cognate cleavage site and the degradation sequence are located at the C-terminus of the second fusion protein.

59. The composition of any one of claims 56-58, wherein the repressible protease is located at the C-terminus of the first fusion protein.

60. The composition of any one of claims 56-59, wherein the first fusion protein further comprises a second intracellular signaling domain.

61. The composition of any one of claims 56-60, wherein the second fusion protein further comprises a third intracellular signaling domain.

62. The composition of any one of claims 56-61, wherein the second fusion protein further comprises an extracellular protein binding domain.

63. The composition of any one of claims 56-62, wherein the first fusion protein and the second fusion protein are bound through the first recruitment domain and the second recruitment domain.

64. The composition of any one of claims 56-63, further comprising a protease inhibitor bound to the repressible protease.

65. A composition comprising:
a. a first fusion protein comprising: (a) an extracellular protein binding domain (b) a first recruitment domain, and (c) a cognate cleavage site; and
b. a second fusion protein comprising: (a′) a second recruitment domain, (b′) a transmembrane domain, and (c′) a repressible protease.

66. The composition of claim 65, wherein:
a. the first fusion protein is a soluble protein;
b. the first fusion protein is a membrane-bound protein comprising a transmembrane domain, and the first recruitment domain is located between the extracellular protein binding domain and the transmembrane domain; or
c. the first fusion protein is a membrane-bound protein comprising a transmembrane domain, and the transmembrane domain is located between the first recruitment domain and the extracellular protein binding domain.

67. The composition of claim 65 or claim 66, wherein:
a. the second fusion protein comprises from the N-terminus to the C-terminus the second recruitment domain, the transmembrane domain, and the repressible protease; or
b. the second fusion protein comprises from the N-terminus to the C-terminus the transmembrane domain, the second recruitment domain, and the repressible protease.

68. The composition of any one of claims 65-67, wherein the first fusion protein is a soluble protein and the cognate cleavage site is located between the extracellular protein binding domain and the first recruitment domain.

69. The composition of any one of claims 65-68, wherein the first fusion protein is a membrane-bound protein comprising a transmembrane domain, wherein the first fusion protein further comprises a first intracellular signaling domain, and the cognate cleavage site is located:
a. between the extracellular protein binding domain and the transmembrane domain;
b. between the transmembrane domain and the first recruitment domain;
c. between the transmembrane domain and the first intracellular signaling domain; or d. between the first recruitment domain and the first intracellular signaling domain.

70. The composition of any one of claims 65-69, wherein the second fusion further comprises a second intracellular signaling domain.

71. The composition of any one of claims 65-70, wherein the second fusion protein further comprises a second extracellular protein binding domain.

72. The composition of any one of claims 65-71, wherein the first fusion protein further comprises a second intracellular signaling domain.

73. The composition of any one of claims 65-72, wherein the first fusion protein and the second fusion protein are bound through the first recruitment domain and the second recruitment domain.

74. The composition of any one of claims 65-73, further comprising a protease inhibitor bound to the repressible protease.

75. A composition comprising:
a. a first fusion protein comprising: (a) an extracellular protein binding domain (b) a transmembrane domain, (c) first recruitment domain, and (d) a self-excising degron, wherein the degron comprises a repressible protease, a cognate cleavage site, and a degradation sequence; and
b. a second fusion protein comprising: (a) a transmembrane domain, (b) a second recruitment domain, and (c) one or more intracellular signaling domains.

76. The composition of claim 75, wherein the self-excising degron is located at the C-terminus of the first fusion protein.

77. The fusion protein or the composition of any one of claims 1-76, wherein the repressible protease is hepatitis C virus (HCV) nonstructural protein 3 (NS3).

78. The fusion protein or the composition of claim 77, wherein the cognate cleavage site comprises an NS3 protease cleavage site.

79. The fusion protein or the composition of claim 78, wherein the NS3 protease cleavage site comprises a NS3/NS4A, a NS4A/NS4B, a NS4B/NSSA, or a NSSA/NSSB junction cleavage site.

80. The fusion protein or the composition of any one of claims 11, 25, 42, 55, 64, and 74, wherein the protease inhibitor is selected from the group consisting of simeprevir, danoprevir, asunaprevir, ciluprevir, boceprevir, sovaprevir, paritaprevir and telaprevir.

81. The fusion protein or the composition of any one of claims 1-80, wherein the degradation sequence is at least 90% identical to the sequence identified by SEQ ID NO: 1.

82. The fusion protein or the composition of claim 81, wherein the degradation sequence comprises the sequence identified by SEQ ID NO: 1.

83. The fusion protein or the composition of any one of claims 1-82, wherein the first intracellular signaling domain comprises CD3zeta, CD28, ZAP40, 4-1BB (CD137), CD28, ICOS, BTLA, OX-40, CD27, CD30, GITR, HVEM, DAP10, DAP12, CD2, MyD88, or a fragment thereof.

84. The fusion protein or the composition of any one of claims 1-82, wherein the first signaling domain comprises immunoreceptor tyrosine-based activation motif (ITAM).

85. The fusion protein or the composition of any one of claims 1-84, comprising a second intracellular signaling domain, wherein the second intracellular signaling domain comprises CD3zeta, CD28, ZAP40, 4-1BB (CD137), CD28, ICOS, BTLA, OX-40, CD27, CD30, GITR, HVEM, DAP10, DAP12, CD2, MyD88, or a fragment thereof.

86. The fusion protein or the composition of any one of claims 1-84, comprising a second intracellular signaling domain, wherein the second intracellular signaling domain comprises immunoreceptor tyrosine-based activation motif (ITAM).

87. The fusion protein or the composition of any one of claims 1-86, comprising a third intracellular signaling domain, wherein the third intracellular signaling domain comprises CD3zeta, CD28, ZAP40, 4-1BB (CD137), CD28, ICOS, BTLA, OX-40, CD27, CD30, GITR, HVEM, DAP10, DAP12, CD2, MyD88, or a fragment thereof.

88. The fusion protein or the composition of any one of claims 1-86, comprising a third intracellular signaling domain, wherein the third intracellular signaling domain comprises immunoreceptor tyrosine-based activation motif (ITAM).

89. The fusion protein or the composition of any one of claims 1-88, wherein the extracellular protein binding domain comprises an antibody, or a fragment thereof.

90. The fusion protein or the composition of claim 89, wherein the extracellular protein binding domain comprises a scFv.

91. The fusion protein or the composition of any one of claims 1-88, wherein the extracellular protein binding domain comprises a ligand-receptor.

92. The composition of any one of claims 29-91, wherein the first and second recruitment domains are pairs of constitutive protein interaction domains selected from the group consisting of (a) cognate leucine zipper domains, (b) cognate PSD95- Dlgl-zo-1 (PDZ) domains, (c) a streptavidin domain and cognate streptavidin binding protein (SBP) domain, (d) a PYL domain and cognate ABI domain, (e) a pair of cognate zinc finger domains, (f) a pair of cognate SH3 domains, and (g) a peptide and antibody or antigen-binding fragment thereof that specifically binds to the peptide.

93. The composition of claim 92, wherein the peptide is selected from the group consisting of: peptide neoepitopes (PNEs), naturally occurring peptides, non-human peptides, yeast peptides, synthetic peptide tags, peptide nucleic acid (PNA), a SunTags, myc-tags, His-tags, HA-tags, peridinin chlorophyll protein complex, green fluorescent protein (GFP), red fluorescent protein (RFP), phycoerythrin (PE), streptavidin, avidin, horse radish peroxidase (HRP), alkaline phosphatase, glucose oxidase, glutathione-S-transferase (GST), maltose binding protein, V5, VSVG, softag 1, softag 3, express tag, S tag, palmitoylation, nitrosylation, SUMO tags, thioredoxin, polyfNANP, poly-Arg, calmodulin binding proteins, PurF fragment, ketosteroid isomerase, PaP3.30, TAF12 histone fold domains, FKBP-tags, SNAP tags, Halo-tags, peptides from RNAse I, small linear hydrophilic peptides, short linear epitopes, and short linear epitope from human nuclear La protein (E5B9).

94. The composition of any one of claims 29-91, wherein the first and second recruitment domains are pairs of constitutive protein interaction domains selected from the group consisting of a pair of cognate leucine zipper domains, a pair of cognate PSD95- Dlgl-zo-1 (PDZ) domains, a streptavidin domain and cognate streptavidin binding protein (SBP) domain, a PYL domain and cognate ABI domain, a pair of cognate zinc finger domains, a pair of cognate SH3 domains, and a peptide and antibody, or antigen-binding fragment thereof, that specifically binds to the peptide.

95. The composition of any one of claims 29-91, wherein the first recruitment domain comprises: FK506 binding protein (FKBP); calcineurin catalytic subunit A (CnA);
cyclophilin; FKBP-rapamycin associated protein (FRB); gyrase B (GyrB);
dihydrofolate reductase (DHFR); DmrB; PYL; ABI; Cry2; CIP; GAI; GID1; or a fragment thereof.

96. The composition of any one of claims 29-91, wherein the second recruitment domain comprises: FK506 binding protein (FKBP); calcineurin catalytic subunit A (CnA);
cyclophilin; FKBP-rapamycin associated protein (FRB); gyrase B (GyrB);
dihydrofolate reductase (DHFR); DmrB; PYL; ABI; Cry2; CIP; GAI; GID1; or a fragment thereof.

97. A polynucleotide encoding the fusion protein of any one of claims 1-28 and 77-91.

98. A vector comprising the polynucleotide of claim 97.

99. A set of polynucleotides comprising:
a. a first polynucleotide encoding the first fusion protein of any one of claims 29-96; and
b. a second polynucleotide encoding the second fusion protein of any one of claims 29-96.

100. A set of vectors comprising:
a. a first vector comprising the first polynucleotide of claim 99; and
b. a second vector comprising the second polynucleotide of claim 99.

101. A cell comprising the fusion protein or the composition of any one of claims 1-96.

102. The cell of claim 101, wherein the cell is an immune cell or a cell line derived from an immune cell.

103. The cell of claim 102, wherein the immune cell is selected from the group consisting of a T cell, a B cell, an NK cell, an NKT cell, an innate lymphoid cell, a mast cell, an eosinophil, a basophils, a macrophage, a neutrophil, a dendritic cell, and any combinations thereof.

104. The cell of claim 101, wherein the cell is a mesenchymal stem cell.

105. A pharmaceutical composition comprising the fusion protein or the composition of any one of claims 1-96, and an excipient.

106. A pharmaceutical composition comprising the cell of any one of claims 101-104 and an excipient.

107. A method of regulating activity of a chimeric antigen receptor (CAR), comprising the steps of:
a. providing a population of cells comprising the fusion protein or the composition of any one of claims 1-96, and
b. contacting the population of cells with a protease inhibitor.

108. The method of claim 107, wherein at least 80%, at least 85%, at least 90%, at least 95%, or at least 98% of the population of cells is activated in response to a ligand to the extracellular protein binding domain, prior to the contacting step.

109. The method of claim 107or claim 108, wherein at least 75% of the population of cells is inactivated following the contacting step.

110. The method of claim 109, wherein less than 25% of the population of cells is activated following the contacting step.

111. The method of claim 107, wherein the population of cells is provided with the fusion protein or the composition of any one of claims 1-12, 40, 43-55, and the step of contacting the population of cells with a protease inhibitor induces the CAR to be degraded.

112. The method of claim 111, wherein the step of contacting induces at least 60%, 70%, 80%, 90%, 95%, 96%, 97%, 98%, or 99% of the CAR to be degraded.

113. The method of claim 107, wherein the population of cells is provided with the fusion protein or the composition of any one of claims 65-74, and the step of contacting the population of cells with a protease inhibitor prevents degradation of the CAR.

114. The method of claim 113, wherein after the step of contacting, degradation of at least 60%, 70%, 80%, 90%, 95%, 96%, 97%, 98%, or 99% of the CAR is prevented compared to before the step of contacting.

115. The method of any one of claims 107-114, further comprising the step of removing the protease inhibitor from the population of cells.

116. The method of any one of claims 107-115, further comprising the step of administering the population of cells to a subject in need of a cell-based therapy.

117. A method of treating a subject in need of a cell-based therapy comprising the step of:

118. administering to the subject a population of cells comprising the fusion protein or the composition of any one of claims 1-96.

119. The method of claim 117, wherein the population of cells was cultured in the presence of a protease inhibitor capable of inhibiting the repressible protease.

120. The method of claim 117, wherein the population of cells was cultured in the absence of a protease inhibitor capable of inhibiting the repressible protease.

121. The method of any one of claims 117-120, further comprising the step of administering to the subject the protease inhibitor capable of inhibiting the repressible protease.

122. The method of claim 121, further comprising the step of withdrawing the protease inhibitor capable of inhibiting the repressible protease from the subject.

123. A method of preparing a population of therapeutic cells, comprising the steps of:
a. providing a population of cells comprising a polynucleotide or a set of polynucleotides encoding the fusion protein or the composition of any one of claims 1-96; and
b. culturing the population of cells, thereby obtaining the population of therapeutic cells.

124. The method of claim 123, wherein the population of therapeutic cells comprises the fusion protein or the composition of any one of claims 1-96.

125. The method of claim 124, further comprising the step of:
a. delivering the polynucleotide encoding the fusion protein of any one of claims 1-25 to a population of naïve cells, thereby obtaining the population of cells; or
b. delivering the set of polynucleotides comprising a first polynucleotide encoding the first fusion protein of any one of claims 29-96; and a second polynucleotide encoding the second fusion protein of any one of claims 29-96 to a population of naïve cells, thereby obtaining the population of cells.

126. The method of any one of claims 123-125, wherein the culturing step is performed in the presence of a protease inhibitor capable of inhibiting the repressible protease.

127. The method of any one of claims 123-125, wherein the culturing step is performed in the absence of a protease inhibitor capable of inhibiting the repressible protease.
a. The method of any one of claims 123-127, further comprising the step of adding an excipient to the population of therapeutic cells.


SEQUENCE LISTING (34 sequences)

SEQ ID NO: 1 - protein, 42 aa, Unknown
  source: /note=“Description of Unknown Degradation sequence”
PITKIDTKYIMTCMSADLEVVTSTWVLVGGVLAALAAYCLST

SEQ ID NO: 2 - protein, 10 aa, Hepacivirus C
DEMEECSQHL

SEQ ID NO: 3 - protein, 9 aa, Hepacivirus C
EDVVPCSMG

SEQ ID NO: 4 - protein, 304 aa, Artificial Sequence
  source: /note=“Description of Artificial Sequence Synthetic polypeptide”
DEMEECSQHLPGAGSSGDIMDYKDDDDKGSSGTGSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDTKYIMTCMSADLEVVTSTWVLVGGVLAALAAYCLSTGCVVIVGRIVLSGKPAIIPDREVLY

SEQ ID NO: 5 - protein, 303 aa, Artificial Sequence
  source: /note=“Description of Artificial Sequence Synthetic polypeptide”
MDYKDDDDKGSSGTGSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDTKYIMTCMSADLEVVTSTWVLVGGVLAALAAYCLSTGCVVIVGRIVLSGKPAGSSGSSIIPDREVLYQEFEDVVPCSMG

SEQ ID NO: 6 - protein, 186 aa, Hepacivirus C
APITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTD

SEQ ID NO: 7 - protein, 99 aa, Human immunodeficiency virus
PQVTLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNF

SEQ ID NO: 8 - protein, 619 aa, Dengue virus 1
SGVLWDTPSPPEVERAVLDDGIYRIMQRGLLGRSQVGVGVFQDGVFHTMWHVTRGAVLMYQGKRLEPSWASVKKDLISYGGGWRFQGSWNTGEEVQVIAVEPGKNPKNVQTAPGTFKTPEGEVGAIALDFKPGTSGSPIVNREGKIVGLYGNGVVTTSGTYVSAIAQAKASQEGPLPEIEDEVFRKRNLTIMDLHPGSGKTRRYLPAIVREAIRRNVRTLILAPTRVVASEMAEALKGMPIRYQTTAVKSEHTGKEIVDLMCHATFTMRLLSPVRVPNYNMIIMDEAHFTDPASIARRGYISTRVGMGEAAAIFMTATPPGSVEAFPQSNAVIQDEERDIPERSWNSGYEWITDFPGKTVWFVPSIKSGNDIANCLRKNGKRVIQLSRKTFDTEYQKTKNNDWDYVVTTDISEMGANFRADRVIDPRRCLKPVILKDGPERVILAGPMPVTVASAAQRRGRIGRNQNKEGDQYVYMGQPLNNDEDHAHWTEAKMLLDNINTPEGIIPALFEPEREKSAAIDGEYRLRGEARKTFVELMRRGDLPVWLSYKVASEGFQYSDRRWCFDGERNNQVLEENMDVEMWTKEGERKKLRPRWLDARTYSDPLALREFKEFAAGRR

SEQ ID NO: 9 - protein, 618 aa, Dengue virus 1
AGVLWDVPSPPPVGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRKLTIMDLHPGAGKTKRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAGIFMTATPPGSRDPFPQSNAPIMDEEREIPERSWSSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGIKNNQILEENVEVEIWTKEGERKKLKPRWLDAKIYSDPLALKEFKEFAAGRK

SEQ ID NO: 10 - protein, 619 aa, Dengue virus 1
SGVLWDVPSPPETQKAELEEGVYRIKQQGIFGKTQVGVGVQKEGVFHTMWHVTRGAVLTHNGKRLEPNWASVKKDLISYGGGWRLSAQWQKGEEVQVIAVEPGKNPKNFQTMPGIFQTTTGEIGAIALDFKPGTSGSPIINREGKVVGLYGNGVVTKNGGYVSGIAQTNAEPDGPTPELEEEMFKKRNLTIMDLHPGSGKTRKYLPAIVREAIKRRLRTLILAPTRVVAAEMEEALKGLPIRYQTTATKSEHTGREIVDLMCHATFTMRLLSPVRVPNYNLIIMDEAHFTDPASIAARGYISTRVGMGEAAAIFMTATPPGTADAFPQSNAPIQDEERDIPERSWNSGNEWITDFVGKTVWFVPSIKAGNDIANCLRKNGKKVIQLSRKTFDTEYQKTKLNDWDFVVTTDISEMGANFKADRVIDPRRCLKPVILTDGPERVILAGPMPVTVASAAQRRGRVGRNPQKENDQYIFMGQPLNKDEDHAHWTEAKMLLDNINTPEGIIPALFEPEREKSAAIDGEYRLKGESRKTFVELMRRGDLPVWLAHKVASEGIKYTDRKWCFDGERNNQILEENMDVEIWTKEGEKKKLRPRWLDARTYSDPLALKEFKDFAAGRK

SEQ ID NO: 11 - protein, 618 aa, Dengue virus 1
SGALWDVPSPAATQKAALSEGVYRIMQRGLFGKTQVGVGIHIEGVFHTMWHVTRGSVICHETGRLEPSWADVRNDMISYGGGWRLGDKWDKEEDVQVLAIEPGKNPKHVQTKPGLFKTLTGEIGAVTLDFKPGTSGSPIINRKGKVIGLYGNGVVTKSGDYVSAITQAERIGEPDYEVDEDIFRKKRLTIMDLHPGAGKTKRILPSIVREALKRRLRTLILAPTRVVAAEMEEALRGLPIRYQTPAVKSEHTGREIVDLMCHATFTTRLLSSTRVPNYNLIVMDEAHFTDPSSVAARGYISTRVEMGEAAAIFMTATPPGTTDPFPQSNSPIEDIEREIPERSWNTGFDWITDYQGKTVWFVPSIKAGNDIANCLRKSGKKVIQLSRKTFDTEYPKTKLTDWDFVVTTDISEMGANFRAGRVIDPRRCLKPVILPDGPERVILAGPIPVTPASAAQRRGRIGRNPAQEDDQYVFSGDPLKNDEDHAHWTEAKMLLDNIYTPEGIIPTLFGPEREKTQAIDGEFRLRGEQRKTFVELMRRGDLPVWLSYKVASAGISYKDREWCFTGERNNQILEENMEVEIWTREGEKKKLRPKWLDARVYADPMALKDFKEFASGRK

SEQ ID NO: 12 - protein, 64 aa, Homo sapiens
LQMLPESEDEESYDTESEFTEFTEDELPYDDGSLQMLPESEDEESYDTESEFTEFTEDELPYDD

SEQ ID NO: 13 - protein, 57 aa, Homo sapiens
EIKDKEEVQRKRQKLMPNFSDSFGGGSGAGAGGGGMFGSGGGGGGTGSTGPGYSFPH

SEQ ID NO: 14 - protein, 86 aa, Unknown
  source: /note=“Description of Unknown Yeast sequence”
IDDENGSVILQDDDYDDGNNHIPFEDDDVYNYNDNDDDDERIEFEDDDDDDDDSIDNDSVMDRKQPHKAEDESEDVEDVERVSKKD

SEQ ID NO: 15 - protein, 41 aa, Artificial Sequence
  source: /note=“Description of Artificial Sequence Synthetic polypeptide”
PESMREEYRKEGSKRIKCPDCEPFCNKRGSPESMREEYRKE

SEQ ID NO: 16 - protein, 68 aa, Artificial Sequence
  source: /note=“Description of Artificial Sequence Synthetic polypeptide”
RSYSPTSPNYSPTSPSGSYSPTSPNYSPTSPSGGSRSYSPTSPNYSPTSPSGSYSPTSPNYSPTSPSG

SEQ ID NO: 17 - protein, 59 aa, Influenza A virus
PESMREEYRKEGSSLLTEVETPGSPESMREEYRKEGSSLLTEVETPGSPESMREEYRKE

SEQ ID NO: 18 - protein, 51 aa, Influenza A virus
LIEEVRHRLKTTENSGSLIEEVRHRLKTTENSGSLIEEVRHRLKTTENSGS

SEQ ID NO: 19 - protein, 37 aa, Unknown
  source: /note=“Description of Unknown Ornithine decarboxylase sequence”
FPPEVEEQDDGTLPMSCAQESGMDRHPAACASARINV

SEQ ID NO: 20 - protein, 40 aa, Artificial Sequence
  source: /note=“Description of Artificial Sequence Synthetic polypeptide”
SHGFPPEVEEQAAGTLPMSCAQESGMDRHPAACASARINV

SEQ ID NO: 21 - protein, 1306 aa, Unknown
  source: /note=“Description of Unknown Angiotensin converting enzyme sequence”
MGAASGRRGPGLLLPLPLLLLLPPQPALALDPGLQPGNFSADEAGAQLFAQSYNSSAEQVLFQSVAASWAHDTNITAENARRQEEAALLSQEFAEAWGQKAKELYEPIWQNFTDPQLRRIIGAVRTLGSANLPLAKRQQYNALLSNMSRIYSTAKVCLPNKTATCWSLDPDLTNILASSRSYAMLLFAWEGWHNAAGIPLKPLYEDFTALSNEAYKQDGFTDTGAYWRSWYNSPTFEDDLEHLYQQLEPLYLNLHAFVRRALHRRYGDRYINLRGPIPAHLLGDMWAQSWENIYDMVVPFPDKPNLDVTSTMLQQGWNATHMFRVAEEFFTSLELSPMPPEFWEGSMLEKPADGREVVCHASAWDFYNRKDFRIKQCTRVTMDQLSTVHHEMGHIQYYLQYKDLPVSLRRGANPGFHEAIGDVLALSVSTPEHLHKIGLLDRVTNDTESDINYLLKMALEKIAFLPFGYLVDQWRWGVFSGRTPPSRYNFDWWYLRTKYQGICPPVTRNETHFDAGAKFHVPNVTPYIRYFVSFVLQFQFHEALCKEAGYEGPLHQCDIYRSTKAGAKLRKVLQAGSSRPWQEVLKDMVGLDALDAQPLLKYFQPVTQWLQEQNQQNGEVLGWPEYQWHPPLPDNYPEGIDLVTDEAEASKFVEEYDRTSQVVWNEYAEANWNYNTNITTETSKILLQKNMQIANHTLKYGTQARKFDVNQLQNTTIKRIIKKVQDLERAALPAQELEEYNKILLDMETTYSVATVCHPNGSCLQLEPDLTNVMATSRKYEDLLWAWEGWRDKAGRAILQFYPKYVELINQAARLNGYVDAGDSWRSMYETPSLEQDLERLFQELQPLYLNLHAYVRRALHRHYGAQHINLEGPIPAHLLGNMWAQTWSNIYDLVVPFPSAPSMDTTEAMLKQGWTPRRMFKEADDFFTSLGLLPVPPEFWNKSMLEKPTDGREVVCHASAWDFYNGKDFRIKQCTTVNLEDLVVAHHEMGHIQYFMQYKDLPVALREGANPGFHEAIGDVLALSVSTPKHLHSLNLLSSEGGSDEHDINFLMKMALDKIAFIPFSYLVDQWRWRVFDGSITKENYNQEWWSLRLKYQGLCPPVPRTQGDFDPGAKFHIPSSVPYIRYFVSFIIQFQFHEALCQAAGHTGPLHKCDIYQSKEAGQRLATAMKLGFSRPWPEAMQLITGQPNMSASAMLSYFKPLLDWLRTENELHGEKLGWPQYNWTPNSARSEGPLPDSGRVSFLGLDLDAQQARVGQWLLLFLGIALLVATLGLSQRLFSIRHRSLHRHSHGPQFGSEVELRHS

SEQ ID NO: 22 - protein, 8 aa, Artificial Sequence
  source: /note=“Description of Artificial Sequence Synthetic peptide”
EPLFAERK

SEQ ID NO: 23 - protein, 4 aa, Unknown
  source: /note=“Description of Unknown Cognate cleavage site sequence”
YVAD

SEQ ID NO: 24 - protein, 5 aa, Unknown
  source: /note=“Description of Unknown Cognate cleavage site sequence”
VDVAD

SEQ ID NO: 25 - protein, 4 aa, Unknown
  source: /note=“Description of Unknown Cognate cleavage site sequence”
DEVD

SEQ ID NO: 26 - protein, 4 aa, Unknown
  source: /note=“Description of Unknown Cognate cleavage site sequence”
VEHD

SEQ ID NO: 27 - protein, 4 aa, Unknown
  source: /note=“Description of Unknown Cognate cleavage site sequence”
LGHD

SEQ ID NO: 28 - protein, 5 aa, Unknown
  source: /note=“Description of Unknown Cognate cleavage site sequence”
LQTDG

SEQ ID NO: 29 - protein, 8 aa, Unknown
  source: /note=“Description of Unknown Cognate cleavage site sequence”
EVNLDAEF

SEQ ID NO: 30 - protein, 7 aa, Unknown
  source: /note=“Description of Unknown Cognate cleavage site sequence”
PQGIAGQ

SEQ ID NO: 31 - protein, 7 aa, Unknown
  source: /note=“Description of Unknown Cognate cleavage site sequence”
ENLYFQS

SEQ ID NO: 32 - protein, 6 aa, Artificial Sequence
  source: /note=“Description of Artificial Sequence Synthetic peptide”
  SITE (6)..(6): Lys(DNP)
HPFHLK

SEQ ID NO: 33 - protein, 11 aa, Unknown
  source: /note=“Description of Unknown Cognate cleavage site sequence”
  VARIANT (2)..(2): /replace=“Gln”
  VARIANT (3)..(3): /replace=“Ala”
  VARIANT (4)..(4): /replace=“Tyr”
  VARIANT (5)..(5): /replace=“Val” or “Met” or “Arg”
  VARIANT (6)..(6): /replace=“Lys”
  VARIANT (8)..(8): /replace=“Ala”
  SITE (1)..(11): /note=“Variant residues given in the sequence have no preference with respect to those in the annotations for variant positions”
AANLTRGGELR

SEQ ID NO: 34 - protein, 6 aa, Unknown
  source: /note=“Description of Unknown Phospho-dependent degron sequence”
  SITE (4)..(5): Any amino acid
DSGXXS